(19) United States
(12) Patent Application Publication
(10) Pub. No.: US 2021/0386069 A1
(43) Pub. Date: Dec. 16, 2021

(54) PYRIDYLPHENYLAMINOQUINOLINES AND ANALOGUES

(71) Applicant: Bayer Aktiengesellschaft, Leverkusen (DE)

(72) Inventors: Pierre CRISTAU, Lyon (FR); Philippe DESBORDES, Lyon (FR); Jeremy DUFOUR, Lyon (FR); Mathieu GOURGUES, Lyon (FR); Sybille LAMPRECHT, Leverkusen (DE); Dominique LOQUE, Vernier (CH); Ruth MEISSNER, Leverkusen (DE); Sebastien NAUD, Collonges au Mont d'Or (FR); Vincent THOMAS, Lyon (FR)

(21) Appl. No.: 17/285,840

(22) PCT Filed: Oct. 17, 2019
(86) PCT No.: PCT/EP2019/078260

§ 371 Date: Apr. 15, 2021

(30) Foreign Application Priority Data
Oct. 18, 2018 (EP) 18201297.1

(51) Int. Cl. A01N 43/42; C07D 401/12; C07D 401/10; C07D 409/14; C07D 401/14; A01N 43/60; A01N 43/58; A01P 3/00

(52) U.S. Cl. CPC A01N 43/42; C07D 401/12; C07D 401/10; A01P 3/00; C07D 401/14; A01N 43/60; A01N 43/58; C07D 409/14

(57) ABSTRACT

The present disclosure relates to fungicidal active compounds, more specifically to pyridylphenylaminoquinolines and analogues thereof, processes and, intermediates for their preparation and use thereof as fungicidal active compound, particularly in the form of fungicide compositions. The present disclosure also relates to methods for the control of phytopathogenic fungi of plants using these compounds or compositions comprising thereof.

### TECHNICAL FIELD

The present disclosure relates to fungicidal active compounds, more specifically to pyridylphenylaminoquinolines and analogues thereof, processes and intermediates for their preparation and use thereof as fungicidal active compounds, particularly in the form of fungicide compositions. The present disclosure also relates to methods for the control of phytopathogenic fungi of plants using these compounds or compositions comprising thereof.

### BACKGROUND

WO 2011/081174 and WO 2012/161071 disclose nitrogen-containing heterocyle compounds suitable for use as fungicides.

WO 2013/058256 discloses further nitrogen-containing heterocyle compounds suitable for use as fungicides.

However, since the ecological and economic demands made on fungicide active compounds are increasing constantly, for example with respect to activity spectrum, toxicity, selectivity, application rate, formation of residues and favourable manufacture, and since there can also be problems associated with resistances, there is a constant need to develop novel fungicidal compounds and compositions which have advantages over the known compounds and compositions at least in some of these aspects.

### DETAILED DESCRIPTION

Accordingly, the present invention provides pyridylphenylaminoquinolines and analogues thereof as described herein below that may be used as microbicide, preferably as fungicide.

### Active Ingredients

The present invention provides compounds of formula (I)

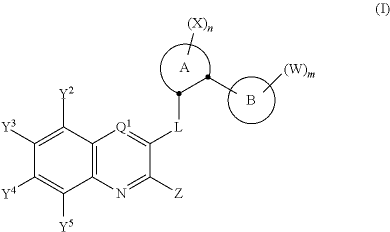

wherein
A is a phenyl or a 5- or 6-membered unsaturated heterocyclyl ring comprising 1, 2 or 3 heteroatoms independently selected in the list consisting of N, O and S, wherein the two points of attachment of the ring A respectively to the group B and to the group L, are adjacent carbon atoms (denoted as •);
B is a partially saturated or unsaturated 6-membered heterocyclyl ring comprising 1, 2, 3 or 4 heteroatoms independently selected in the list consisting of N, O and S;
Q¹ is CY¹ or N wherein:
   Y¹ is selected from the group consisting of hydrogen atom, halogen atom, C₁-C₈-alkyl, C₁-C₈-halogenoalkyl comprising up to 9 halogen atoms that can be the same or different, C₂-C₈-alkenyl, C₂-C₈-halogenoalkenyl comprising up to 9 halogen atoms that can be the same or different, C₂-C₈-alkynyl, C₂-C₈-halogenoalkynyl comprising up to 9 halogen atoms that can be the same or different, C₃-C₇-cycloalkyl, C₄-C₇-cycloalkenyl, hydroxyl, C₁-C₈-alkoxy, C₁-C₈-halogenoalkoxy comprising up to 9 halogen atoms that can be the same or different, formyl, amino, C₁-C₈-alkylamino, di-C₁-C₈-alkylamino, sulfanyl, C₁-C₈-alkylsulfanyl, C₁-C₈-alkylsulfinyl, C₁-C₈-alkylsulfonyl, C₁-C₆-trialkylsilyl, cyano and nitro,
   wherein said C₃-C₇-cycloalkyl and C₄-C₇-cycloalkenyl may be substituted with one or more Y^{a} substituents;
Y², Y³, Y⁴ and Y⁵ are independently selected from the group consisting of hydrogen atom, halogen atom, C₁-C₈-alkyl, C₁-C₈-halogenoalkyl comprising up to 9 halogen atoms that can be the same or different, C₂-C₈-alkenyl, C₂-C₈-halogenoalkenyl comprising up to 9 halogen atoms that can be the same or different, C₂-C₈-alkynyl, C₂-C₈-halogenoalkynyl comprising up to 9 halogen atoms that can be the same or different, C₃-C₇-cycloalkyl, C₄-C₇-cycloalkenyl, hydroxyl, C₁-C₈-alkoxy, C₁-C₈-halogenoalkoxy comprising up to 9 halogen atoms that can be the same or different, formyl, amino, C₁-C₈-alkylamino, di-C₁-C₈-alkylamino, sulfanyl, C₁-C₈-alkylsulfanyl, C₁-C₈-alkylsulfinyl, C1-C₈-alkylsulfonyl, C₁-C₆-trialkylsilyl, cyano and nitro,
wherein said C₃-C₇-cycloalkyl and C₄-C₇-cycloalkenyl may be substituted with one or more Y^{a} substituents;
Z is selected from the group consisting of hydrogen atom, halogen atom, hydroxyl, C₁-C₈-alkyl, C₂-C₈-alkenyl, C₂-C₈-alkynyl, C₂-C₈-halogenoalkynyl comprising up to 9 halogen atoms that can be the same or different, C₁-C₈-alkoxy, C₁-C₈-halogenoalkyl comprising up to 9 halogen atoms that can be the same or different, C₂-C₈-halogenoalkenyl comprising up to 9 halogen atoms that can be the same or different, C₁-C₈-halogenoalkoxy comprising up to 9 halogen atoms that can be the same or different, C₃-C₇-cycloalkyl, C₄-C₇-cycloalkenyl, formyl, C₁-C₈-alkylamino, di-C₁-C₈-alkylamino, sulfanyl, C₁-C₈-alkylsulfanyl, C₁-C₈-alkylsulfinyl, C₁-C₈-alkylsulfonyl, C₁-C₆-trialkylsilyl, cyano and nitro,
wherein said C₃-C₇-cycloalkyl and C₄-C₇-cycloalkenyl may be substituted with one or more Z^{a} substituents;
m is 0, 1, 2, 3 or 4;
n is 0, 1, 2, 3 or 4;
L is CR¹R² or NR³ wherein
   R¹ and R² are independently selected from the group consisting of hydrogen atom, halogen atom, C₁-C₈-alkoxy and C₁-C₈ alkyl,
   R³ is selected from the group consisting of hydrogen atom, C₁-C₈-alkyl, C₁-C₈-halogenoalkyl comprising up to 9 halogen atoms that can be the same or different, C₂-C₈-alkenyl, C₂-C₈-halogenoalkenyl comprising up to 9 halogen atoms that can be the same or different, C₃-C₈-alkynyl, C₃-C₈-halogenoalkynyl comprising up to 9 halogen atoms that can be the same or different, C₃-C₇-cycloalkyl, C₃-C₇-halogenocycloalkyl comprising up to 9 halogen atoms that can be the same or different, C₃-C₇-cycloalkyl-C₁-C₈-alkyl, C₁-C₈-alkylcarbonyl, C₁-C₈-halogenoalkylcarbonyl comprising up to 9 halogen atoms that can be the same or different, C₁-C₈-alkoxycarbonyl, C₁-C₈-halogenoalkoxycarbonyl comprising up to 9 halogen atoms that can be the same or different, C₁-C₈-alkylsulfonyl, C₁-C₈-halogenoalkylsulfonyl comprising up to 9 halogen atoms that can be the same or different, aryl-C₁-C₈-alkyl and phenylsulfonyl,
   wherein said C₃-C₇-cycloalkyl, C₃-C₇-cycloalkyl-C₁-C₈-alkyl, aryl-C₁-C₈-alkyl and phenylsulfonyl may be substituted with one or more R^{3a} substituents;
W is independently selected from the group consisting of halogen atom, hydroxyl, C₁-C₈-alkyl, C₁-C₈-halogenoalkyl comprising up to 9 halogen atoms that can be the same or different, C₁-C₈-alkoxy, C₁-C₈-halogenoalkoxy comprising up to 9 halogen atoms that can be the same or different, C₁-C₈-hydroxyalkyl, C₁-C₈-alkoxy-C₁-C₈-alkyl, C₂-C₈-alkenyl, C₂-C₈-halogenoalkenyl comprising up to 9 halogen atoms that can be the same or different, C₂-C₈-alkynyl, C₂-C₈-halogenoalkynyl comprising up to 9 halogen atoms that can be the same or different, C₃-C₇-cycloalkyl, C₄-C₈-cycloalkenyl, aryl, aryl-C₁-C₈-alkyl, heterocyclyl, heterocyclyl-C₁-C₈-alkyl, aryloxy, heteroaryloxy, arylsulfanyl, arylsulfinyl, arylsulfonyl, heteroarylsulfanyl, heteroarylsulfinyl, heteroarylsulfonyl, arylamino, heteroarylamino, aryloxy-C₁-C₈-alkyl, heteroaryloxy-C₁-C₈-alkyl, arylsulfanyl-C₁-C₈-alkyl, arylsulfinyl-C₁-C₈-alkyl, arylsulfonyl-C₁-C₈-alkyl, heteroarylsulfanyl-C₁-C₈-alkyl, heteroarylsulfinyl-C₁-C₈-alkyl, heteroarylsulfonyl-C₁-C₈-alkyl, arylamino-C₁-C₈-alkyl, heteroarylamino-C₁-C₈-alkyl, aryl-C₁-C₈-alkoxy, heteroaryl-C₁-C₈-alkoxy, aryl-C₁-C₈-alkylsulfanyl, aryl-C₁-C₈-alkylsulfinyl, aryl-C₁-C₈-alkylsulfonyl, heteroaryl-C₁-C₈-alkylsulfanyl, heteroaryl-C₁-C₈-alkylsulfinyl, heteroaryl-C₁-C₈-alkylsulfonyl, aryl-C₁-C₈-alkylamino, heteroaryl-C₁-C₈-alkylamino, formyl, C₁-C₈-alkylcarbonyl, (hydroxyimino)C₁-C₈-alkyl, (C₁-C₈-alkoxyimino)C₁-C₈-alkyl, carboxyl, C₁-C₈-alkoxycarbonyl, carbamoyl, C₁-C₈-alkylcarbamoyl, di-C₁-C₈-alkylcarbamoyl, amino, C₁-C₈-alkylamino, di-C₁-C₈-alkylamino, sulfanyl, C₁-C₈-alkylsulfanyl, C₁-C₈-alkylsulfinyl, C₁-C₈-alkylsulfonyl, C₁-C₆-trialkylsilyl, tri(C₁-C₈-alkyl)silyloxy, tri(C₁-C₈-alkyl)silyloxy-C₁-C₈-alkyl, oxo, cyano and nitro,
wherein said C₃-C₇-cycloalkyl, C₄-C₈-cycloalkenyl, heterocyclyl, aryl and the aryl, heterocyclyl and heteroaryl moieties of the aryl-C₁-C₈-alkyl, heterocyclyl-C₁-C₈-alkyl, aryloxy, heteroaryloxy, arylsulfanyl, arylsulfinyl, arylsulfonyl, heteroarylsulfanyl, heteroarylsulfinyl, heteroarylsulfonyl, arylamino, heteroarylamino, aryloxy-C₁-C₈-alkyl, heteroaryloxy-C₁-C₈-alkyl, arylsulfanyl-C₁-C₈-alkyl, arylsulfinyl-C₁-C₈-alkyl, arylsulfonyl-C₁-C₈-alkyl, heteroarylsulfanyl-C₁-C₈-alkyl, heteroarylsulfinyl-C₁-C₈-alkyl, heteroarylsulfonyl-C₁-C₈-alkyl, arylamino-C₁-C₈-alkyl, heteroarylamino-C₁-C₈-alkyl, aryl-C₁-C₈-alkoxy, heteroaryl-C₁-C₈-alkoxy, aryl-C₁-C₈-alkylsulfanyl, aryl-C₁-C₈-alkylsulfinyl, aryl-C₁-C₈-alkylsulfonyl, heteroaryl-C₁-C₈-alkylsulfanyl, heteroaryl-C₁-C₈-alkylsulfinyl, heteroaryl-C₁-C₈-alkylsulfonyl, aryl-C₁-C₈-alkylamino, heteroaryl-C₁-C₈-alkylamino groups may be substituted with one or more W^{a} substituents;
X is independently selected from the group consisting of halogen atom, hydroxyl, C₁-C₈-alkyl, C₁-C₈-halogenoalkyl comprising up to 9 halogen atoms that can be the same or different, C₁-C₃-alkoxy, C₁-C₈-halogenoalkoxy comprising up to 9 halogen atoms that can be the same or different, C₂-C₈-alkenyl, C₂-C₈-halogenoalkenyl comprising up to 9 halogen atoms that can be the same or different, C₂-C₈-alkynyl, C₂-C₈-halogenoalkynyl comprising up to 9 halogen atoms that can be the same or different, C₃-C₇-cycloalkyl, C₄-C₇-cycloalkenyl, formyl, amino, C₁-C₈-alkylamino, di-C₁-C₈-alkylamino, sulfanyl, C₁-C₈-alkylsulfanyl, C₁-C₈-alkylsulfinyl, C₁-C₈-alkylsulfonyl, C₁-C₆-trialkylsilyl, cyano, nitro and C₁-C₈-hydroxyalkyl,
wherein said C₃-C₇-cycloalkyl and C₄-C₇-cycloalkenyl may be substituted with one or more X^{a} substituents;
   Z^{a}, R^{3a}, W^{a}, X^{a} and Y^{a} are independently selected from the group consisting of halogen atom, nitro, hydroxyl, cyano, carboxyl, amino, sulfanyl, pentafluoro-X⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₈-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl and C₁-C₈-halogeno-alkyl-sulfonyl having 1 to 5 halogen atoms;
   as well as their salts, N-oxides, metal complexes, metalloid complexes and optically active isomers or geometric isomers;
   provided that compound of formula (I) is not
2-{2-[(6,7-dimethyl-3-oxo-3,4-dihydroquinoxalin-2-yl)methyl]phenyl}-3,5-dioxo-2,3,4,5-tetrahydro-1,2,4-triazine-6-carbonitrile [713527-70-9], and
3,5-dioxo-2-{2-[(3-oxo-3,4-dihydroquinoxalin-2-yl)methyl]phenyl}-2,3,4,5-tetrahydro-1,2,4-triazine-6-carbonitrile [426815-75-0].

As used herein, the expression “one or more substituents” refers to a number of substituents that ranges from one to the maximum number of substituents possible based on the number of available bonding sites, provided that the conditions of stability and chemical feasibility are met.

As used herein, halogen means fluorine, chlorine, bromine or iodine; formyl means —CH(═O); carboxy means —C(═O)OH; carbonyl means —C(═O)—; carbamoyl means —C(═O)NH₂; N-hydroxycarbamoyl means —C(═O)NHOH; SO represents a sulfoxide group; SO₂ represents a sulfone group; heteroatom means sulfur, nitrogen or oxygen; methylene means the diradical —CH₂—; aryl means an organic radical derived from an aromatic hydrocarbon by removal of one hydrogen, such as phenyl or naphthyl; unless provided differently, heterocyclyl means an unsaturated, saturated or partially saturated 5- to 7-membered ring, preferably a 5- to 6-membered ring, comprising from 1 to 4 heteroatoms independently selected in the list consisting of N, O and S. The term “heterocyclyl” as used herein encompasses heteroaryl.

The term “membered” as used herein for instance in the expression “6-membered ring” or “5- to 6-membered ring” designates the number of skeletal atoms that constitutes the ring.

As used herein, an alkyl group, an alkenyl group and an alkynyl group as well as moieties containing these terms, can be linear or branched.

When an amino group or the amino moiety of any other amino-containing group is substituted by two substituents that can be the same or different, the two substituents together with the nitrogen atom to which they are linked can form a heterocyclyl group, preferably a 5- to 7-membered heterocyclyl group, that can be substituted or that can include other hetero atoms, for example a morpholino group or piperidinyl group.

Any of the compounds of the present invention can exist in one or more optical or chiral isomer forms depending on the number of asymmetric centres in the compound. The invention thus relates equally to all optical isomers and racemic or scalemic mixtures thereof (the term “scalemic” denotes a mixture of enantiomers in different proportions) and to mixtures of all possible stereoisomers, in all proportions. The diastereoisomers and/or the optical isomers can be separated according to methods which are known per se by the man ordinary skilled in the art.

Any of the compounds of the present invention can also exist in one or more geometric isomer forms depending on the number of double bonds in the compound. The invention thus relates equally to all geometric isomers and to all possible mixtures, in all proportions. The geometric isomers can be separated according to general methods, which are known per se by the man ordinary skilled in the art.

Any of the compounds of the present invention can also exist in one or more geometric isomer forms depending on the relative position (syn/anti or cis/trans) of the substituents of the chain or ring. The invention thus relates equally to all syn/anti (or cis/trans) isomers and to all possible syn/anti (or cis/trans) mixtures, in all proportions. The syn/anti (or cis/trans) isomers can be separated according to general methods, which are known per se by the man ordinary skilled in the art.

When a compound of the invention can be present in tautomeric form, the invention also encompasses any tautomeric forms of such compound, even when this is not expressly mentioned.

Compounds of formula (I) are herein referred to as “active ingredient(s)”.

In the above formula (I), A is preferably selected from the group consisting of phenyl, thienyl, pyridinyl and pyrimidyl, more preferably A is selected from the group consisting of A-G1, A-G2, A-G3, A-G4, A-G5, A-G6 and A-G7:

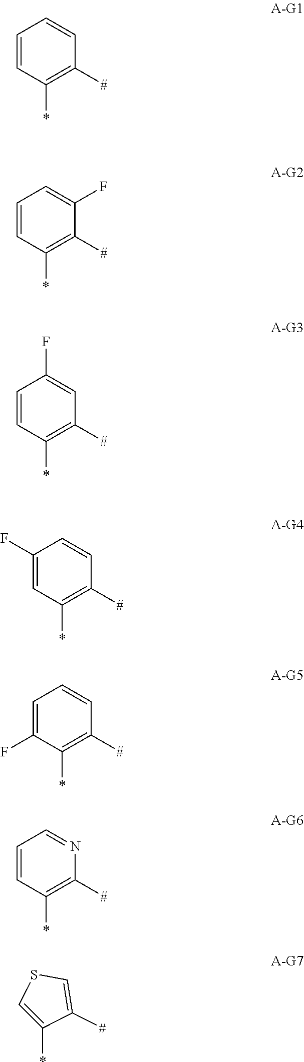

wherein “*” denotes the connection to L and “#” denotes the connection to B.

In some embodiments, A is phenyl, preferably A is selected from the group consisting of A-G1, A-G2, A-G3, A-G4 and A-G5. In some embodiments, A is A-G1, A-G2 or A-G3. In some other embodiments, A is A-G2 or A-G3.

In the above formula (I), B may be a partially saturated or unsaturated 6-membered heterocyclyl ring comprising comprising 1, 2 or 3 heteroatoms chosen from nitrogen atom or oxygen atom (e.g. pyridinyl, pyrimidinyl, pyrazinyl, pyridazinyl, dihydropyranyl, dihydropyridinyl), preferably B is selected from the group consisting of dihydropyranyl, dihydropyridinyl, pyridinyl and pyrimidinyl.

In the above formula (I), B is preferably a partially saturated or unsaturated 6-membered heterocyclyl ring comprising comprising 1, 2 or 3 nitrogen atoms, more preferably B is selected from the group consisting pyridinyl (e.g. pyridin-2-yl, pyridin-3-yl, pyridin-4-yl), pyrimidinyl (pyrimidin-2-yl, pyrimidin-4-yl, pyrimidin-5-yl), pyrazinyl (e.g. pyrazin-2-yl, pyrazin-3-yl), pyridazinyl (e.g. pyridazin-3-yl and pyridazin-4-yl), dihydropyranyl (e.g. 3,4-dihydro-2H-pyran-6-yl) and dihydropyridinyl (e.g. 5,6-dihydropyridin-2-yl), In some embodiments, B is a pyridinyl group or a pyrimidinyl group.

In the above formula (I), Z is preferably selected from the group consisting of hydrogen atom, halogen atom, C₁-C₆-alkyl, C₁-C₆-halogenoalkyl comprising up to 9 halogen atoms that can be the same or different, C1-C₆-alkoxy, C₁-C₆-halogenoalkoxy comprising up to 9 halogen atoms that can be the same or different and cyano. More preferably Z is a hydrogen atom, a halogen atom (e.g. chlorine), a C₁-C₆-alkyl (e.g. methyl) or a C₁-C₆-halogenoalkyl comprising up to 9 halogen atoms that can be the same or different (e.g. difluoromethyl), even more preferably Z is a hydrogen atom or a C₁-C₆-alkyl (e.g. methyl).

In the above formula (I), X is preferably independently selected from the group consisting of halogen atom, C₁-C₆-alkyl, C₁-C₆-halogenoalkyl comprising up to 9 halogen atoms that can be the same or different, hydroxyl, C₁-C₆-alkoxy and C₁-C₆-halogenoalkoxy comprising up to 9 halogen atoms that can be the same or different, more preferably X is a halogen atom (e.g. chlorine, fluorine), C₁-C₆-alkyl (e.g. methyl), C₁-C₆-halogenoalkyl comprising up to 9 halogen atoms that can be the same or different (trifluromethyl) or C₁-C₆-halogenoalkoxy comprising up to 9 halogen atoms that can be the same or different (e.g. trifluoromethoxy), even more preferably X, when present, is a halogen atom (e.g. fluorine).

In the above formula (I), n is preferably 0, 1 or 2, more preferably 0 or 1.

In the above formula (I), n is preferably 0, 1 or 2, more preferably 0 or 1, with X being preferably selected from the group consisting of halogen atom, C₁-C₆-alkyl, C₁-C₆-halogenoalkyl comprising up to 9 halogen atoms that can be the same or different, hydroxyl, C₁-C₆-alkoxy and C1-C₆-halogenoalkoxy comprising up to 9 halogen atoms that can be the same or different, more preferably X is a halogen atom (e.g. chlorine, fluorine), C₁-C₆-alkyl (e.g. methyl), C₁-C₆-halogenoalkyl comprising up to 9 halogen atoms that can be the same or different (trifluromethyl) or C₁-C₆-halogenoalkoxy comprising up to 9 halogen atoms that can be the same or different (e.g. trifluoromethoxy), even more preferably X, when present, is a halogen atom (e.g. fluorine).

In the above formula (I), Q¹ is preferably CY¹ or N wherein Y¹ is selected from the group consisting of hydrogen atom, halogen atom, C₁-C₆-alkyl, C₁-C₆-halogenoalkyl comprising up to 9 halogen atoms that can be the same or different, C₃-C₇-cycloalkyl, hydroxyl, C₁-C₆-alkoxy, C₁-C₆-halogenoalkoxy comprising up to 9 halogen atoms that can be the same or different, C₁-C₆-alkoxycarbonyl, formyl and cyano, wherein said C₃-C₇-cycloalkyl may be substituted with one or more Y^{a} substituents, preferably Y¹ is selected from the group consisting of hydrogen, halogen (e.g. chlorine) and C₁-C₆-alkyl (e.g. methyl), more preferably Y¹ is a hydrogen atom.

In some embodiments, in the above formula (I), Q¹ is CY¹ wherein Y¹ is a hydrogen atom.

In the above formula (I), Y², Y³, Y⁴ and Y⁵ are preferably independently selected from the group consisting of hydrogen atom, halogen atom, C₁-C₆-alkyl, C₁-C₆-halogenoalkyl comprising up to 9 halogen atoms that can be the same or different, C₃-C₇-cycloalkyl, hydroxyl, C₁-C₆-alkoxy, C₁-C₆-halogenoalkoxy comprising up to 9 halogen atoms that can be the same or different, C₁-C₆-alkylcarbonyl, formyl and cyano, wherein said C₃-C₇-cycloalkyl may be substituted with one or more Y^{a} substituents, more preferably Y², Y³, Y⁴ and Y⁵ are independently selected from the group consisting of hydrogen atom, halogen atom, C₁-C₆-alkyl, C₁-C₆-halogenoalkyl comprising up to 9 halogen atoms that can be the same or different (e.g. trifluoromethyl) or a cyano, even more preferably Y², Y³, Y⁴ and Y⁵ are independently a hydrogen atom or a halogen atom (e.g. fluor).

In the above formula (I), W is preferably independently selected from the group consisting of halogen atom (e.g. chlorine, bromine), C₁-C₆-alkyl, C₁-C₆-halogenoalkyl comprising up to 9 halogen atoms that can be the same or different, C₁-C₆-alkoxy (e.g. methoxy), C₁-C₆-hydroxyalkyl, C₁-C₆-alkylsulfanyl, C₂-C₆-alkenyl, C₁-C₆-alkoxycarbonyl, C₃-C₇-cycloalkyl (e.g. cyclopropyl), aryl, aryl-C₁-C₆-alkyl (wherein said aryl may be substituted with one or more halogen atoms), heterocyclyl, carboxyl, tri(C₁-C₆-alkyl)silyloxy-C₁-C₆-alkyl, heteroaryl-C₁-C₆-alkyl, C₁-C₆-alkoxy-C₁-C₆-alkyl, oxo and cyano.

In some embodiments, W is independently selected from the group consisting of halogen atom (e.g. chlorine, bromine), C₁-C₆-alkyl, C₁-C₆-halogenoalkyl comprising up to 9 halogen atoms that can be the same or different, C₁-C₆-alkoxy (e.g. methoxy), C₁-C₆-alkylsulfanyl, C₃-C₇-cycloalkyl (e.g. cyclopropyl), oxo and cyano.

In some embodiments, W is halogen atom or C₁-C₆-alkoxy.

In the above formula (I), m is preferably 0, 1, 2 or 3, more preferably m is 0, 1 or 2, even more preferably m is 0 or 1.

In the above formula (I), m is preferably 0, 1, 2 or 3, more preferably m is 0 or 1, and W is independently selected from the group consisting of halogen atom (e.g. chlorine, bromine), C₁-C₆-alkyl, C₁-C₆-halogenoalkyl comprising up to 9 halogen atoms that can be the same or different, C₁-C₆-alkoxy (e.g. methoxy), C₁-C₆-hydroxyalkyl, C₁-C₆-alkylsulfanyl, C₂-C₆-alkenyl, C₁-C₆-alkoxycarbonyl, C₃-C₇-cycloalkyl (e.g. cyclopropyl), aryl, aryl-C₁-C₆-alkyl (wherein said aryl may be substituted with one or more halogen atoms), heterocyclyl, carboxyl, tri(C₁-C₆-alkyl)silyloxy-C₁-C₆-alkyl, heteroaryl-C₁-C₆-alkyl, C₁-C₆-alkoxy-C₁-C₆-alkyl, oxo and cyano.

In the above formula (I), R¹ is preferably a hydrogen atom or a halogen atom, more preferably R¹ is a hydrogen atom.

In the above formula (I), R² is preferably a hydrogen atom or a halogen atom, more preferably R² is a hydrogen atom.

In the above formula (I), R³ is preferably a hydrogen atom or a substituted or non-substituted C₁-C₆-alkyl, preferably R³ is a hydrogen atom or a methyl group, even more preferably R³ is a hydrogen atom.

In some embodiments, compounds according to the invention are compounds of formula (I) wherein Y² and Y³ are hydrogen atoms and Y⁴ and Y⁵ are halogen atoms.

In some embodiments, compounds according to the invention are compounds of formula (I) wherein Y², Y³ and Y⁴ are hydrogen atoms and Y⁵ is halogen atom.

The above specified definitions of Q¹, Y¹, Y², Y³, Y⁴, Y⁵, R¹, R², R³, Z, L, A, B, X, W, n and m (e.g. broad definitions as well as preferred, more preferred, even more preferred definitions) can be combined in various manners to provide sub-classes of compounds according to the invention.

In some embodiments (referred herein as embodiment (a)), compounds according to the invention are compounds of formula (I) wherein:

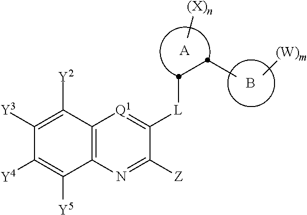

wherein
A is selected from the group consisting of phenyl, thienyl, pyridinyl and pyrimidyl, preferably A is phenyl;
B is a partially saturated or unsaturated 6-membered heterocyclyl ring comprising comprising 1, 2 or 3 heteroatoms chosen from nitrogen atom or oxygen atom, preferably B is selected from the group consisting of dihydropyranyl, dihydropyridinyl, pyridinyl and pyrimidinyl;
Q¹ is CY¹ or N wherein Y¹ is selected from the group consisting of hydrogen atom, halogen atom, C₁-C₆-alkyl, C₁-C₆-halogenoalkyl comprising up to 9 halogen atoms that can be the same or different, C₃-C₇-cycloalkyl, hydroxyl, C₁-C₆-alkoxy, C₁-C₆-halogenoalkoxy comprising up to 9 halogen atoms that can be the same or different, C₁-C₆-alkoxycarbonyl, formyl and cyano, wherein said C₃-C₇-cycloalkyl may be substituted with one or more Y^{a} substituents, preferably Y¹ is selected from the group consisting of hydrogen, halogen (e.g. chlorine) and C₁-C₆-alkyl (e.g. methyl), more preferably Y¹ is a hydrogen atom, more preferably Q¹ is CY¹ wherein Y¹ is a hydrogen atom;
Y², Y³, Y⁴ and Y⁵ are independently selected from the group consisting of hydrogen atom, halogen atom, C₁-C₆-alkyl, C₁-C₆-halogenoalkyl comprising up to 9 halogen atoms that can be the same or different, C₃-C₇-cycloalkyl, hydroxyl, C₁-C₆-alkoxy, C₁-C₆-halogenoalkoxy comprising up to 9 halogen atoms that can be the same or different, C₁-C₆-alkylcarbonyl, formyl and cyano, wherein said C₃-C₇-cycloalkyl may be substituted with one or more Y^{a} substituents, preferably Y², Y³, Y⁴ and Y⁵ are independently selected from the group consisting of hydrogen atom, halogen atom, C₁-C₆-alkyl, C₁-C₆-halogenoalkyl comprising up to 9 halogen atoms that can be the same or different (e.g. trifluoromethyl) or a cyano, even more preferably Y², Y³, Y⁴ and Y⁵ are independently a hydrogen atom or a halogen atom (e.g. fluor);
Z is selected from the group consisting of hydrogen atom, halogen atom, C₁-C₆-alkyl, C₁-C₆-halogenoalkyl comprising up to 9 halogen atoms that can be the same or different, C₁-C₆-alkoxy, C₁-C₆-halogenoalkoxy comprising up to 9 halogen atoms that can be the same or different and cyano, preferably Z is a hydrogen atom, a halogen atom (e.g. chlorine), a C₁-C₆-alkyl (e.g. methyl) or a C₁-C₆-halogenoalkyl comprising up to 9 halogen atoms that can be the same or different (e.g. difluoromethyl), more preferably Z is a hydrogen atom or a C₁-C₆-alkyl (e.g. methyl);
m is 0, 1, 2 or 3, preferably m is 0, 1 or 2, more preferably m is 0 or 1;
n is 0, 1 or 2, preferably 0 or 1;
L is CR¹R² or NR³ wherein R¹ and R² are independently a hydrogen atom or a halogen atom, preferably R¹ and R² are both hydrogen atom, R³ is a hydrogen atom or a substituted or non-substituted C₁-C₆-alkyl, preferably R³ is a hydrogen atom or a methyl group, more preferably R³ is a hydrogen atom; W is independently selected from the group consisting of halogen atom (e.g. chlorine, bromine), C₁-C₆-alkyl, C₁-C₆-halogenoalkyl comprising up to 9 halogen atoms that can be the same or different, C₁-C₆-alkoxy (e.g. methoxy), C₁-C₆-hydroxyalkyl, C₁-C₆-alkylsulfanyl, C₂-C₆-alkenyl, C₁-C₆-alkoxycarbonyl, C₃-C₇-cycloalkyl (e.g. cyclopropyl), aryl, aryl-C₁-C₆-alkyl (wherein said aryl may be substituted with one or more halogen atoms), heterocyclyl, carboxyl, tri(C₁-C₆-alkyl)silyloxy-C₁-C₆-alkyl, heteroaryl-C₁-C₆-alkyl, C₁-C₆-alkoxy-C₁-C₆-alkyl, oxo and cyano, preferably W is selected from the group consisting of halogen atom, C₁-C₆-alkyl, C₁-C₆-halogenoalkyl comprising up to 9 halogen atoms that can be the same or different, C₁-C₆-alkoxy, C₁-C₆-hydroxalkyl, C₂-C₆-alkenyl, C₁-C₆-alkoxycarbonyl, C₃-C₇-cycloalkyl, aryl, aryl-C₁-C₆-alkyl (wherein said aryl may be substituted with one or more halogen atoms), heterocyclyl, carboxyl, tri(C₁-C₆-alkyl)silyloxy-C₁-C₆-alkyl, heteroaryl-C₁-C₆-alkyl and C₁-C₆-alkoxy-C₁-C₆-alkyl;
X is independently selected from the group consisting of halogen atom, C₁-C₆-alkyl, C₁-C₆-halogenoalkyl comprising up to 9 halogen atoms that can be the same or different, hydroxyl, C₁-C₆-alkoxy and C₁-C₆-halogenoalkoxy comprising up to 9 halogen atoms that can be the same or different, preferably X is a halogen atom (e.g. chlorine, fluorine), C₁-C₆-alkyl (e.g. methyl), C₁-C₆-halogenoalkyl comprising up to 9 halogen atoms that can be the same or different (trifluromethyl) or C₁-C₆-halogenoalkoxy comprising up to 9 halogen atoms that can be the same or different (e.g. trifluoromethoxy), more preferably X is a halogen atom (e.g. fluorine).

In some embodiments (referred herein as embodiment (b)), compounds according to the invention are compounds of formula (I) wherein:

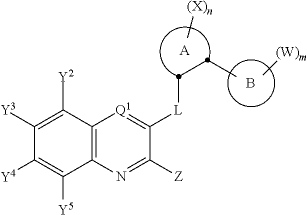

wherein
A is selected from the group consisting of phenyl, thienyl, pyridinyl and pyrimidyl, preferably A is phenyl;
B is a partially saturated or unsaturated 6-membered heterocyclyl ring comprising comprising 1, 2 or 3 heteroatoms chosen from nitrogen atom or oxygen atom, preferably B is selected from the group consisting of dihydropyranyl, dihydropyridinyl, pyridinyl and pyrimidinyl;
Q¹ is N or CY¹ wherein Y¹ is a hydrogen atom;
Y², Y³, Y⁴ and Y⁵ are independently a hydrogen atom or a halogen atom;
Z is a hydrogen atom or a C₁-C₆-alkyl;
m is 0, 1 or 2;
n is 0 or 1;
L is CR¹R² wherein R¹ and R² are hydrogen atoms or L is NR³ wherein R³ is hydrogen atom;
W is independently selected from the group consisting of halogen atom (e.g. chlorine, bromine), C₁-C₆-alkyl, C₁-C₆-halogenoalkyl comprising up to 9 halogen atoms that can be the same or different, C₁-C₆-alkoxy (e.g. methoxy), C₁-C₆-hydroxyalkyl, C₁-C₆-alkylsulfanyl, C₂-C₆-alkenyl, C₁-C₆-alkoxycarbonyl, C₃-C₇-cycloalkyl (e.g. cyclopropyl), aryl, aryl-C₁-C₆-alkyl (wherein said aryl may be substituted with one or more halogen atoms), heterocyclyl, carboxyl, tri(C₁-C₆-alkyl)silyloxy-C₁-C₆-alkyl, heteroaryl-C₁-C₆-alkyl, C₁-C₆-alkoxy-C₁-C₆-alkyl, oxo and cyano, more preferably W is independently selected from the group consisting of halogen atom (e.g. chlorine, bromine), C₁-C₆-alkyl, C₁-C₆-halogenoalkyl comprising up to 9 halogen atoms that can be the same or different, C₁-C₆-alkoxy (e.g. methoxy), C₁-C₆-alkylsulfanyl, C₃-C₇-cycloalkyl (e.g. cyclopropyl), oxo and cyano;
X is halogen atom.

In some embodiments in accordance with embodiment (a) or (b), compounds according to the invention are compounds of formula (I) wherein A is selected from the group consisting of A-G1, A-G2, A-G3, A-G4, A-G5, A-G6 and A-G7, preferably A-G1, A-G2, A-G3, A-G4 and A-G5.

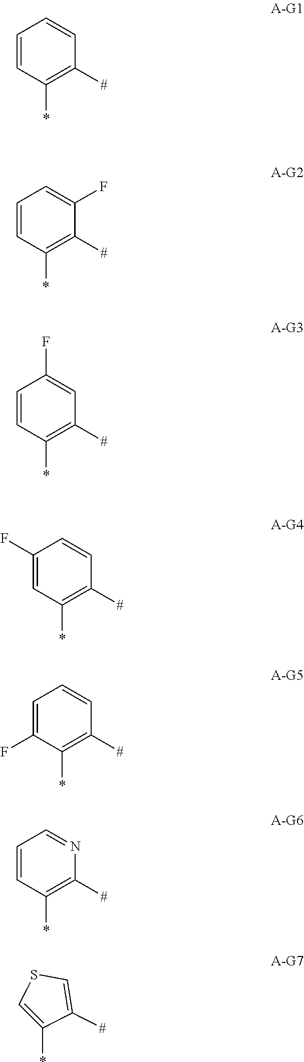

wherein “*” denotes the connection to L and “#” denotes the connection to B.

In some embodiments in accordance with embodiment (a) or (b), compounds according to the invention are compounds of formula (I) wherein A is A-G1, A-G2 or A-G3.

In some embodiments in accordance with embodiment (a) or (b), compounds according to the invention are compounds of formula (I) wherein L is NR³ wherein R³ is a hydrogen atom.

In some embodiments in accordance with embodiment (a) or (b), compounds according to the invention are compounds of formula (I) wherein Y² and Y³ are hydrogen atoms and Y⁴ and Y⁵ are halogen atoms.

In some embodiments in accordance with embodiment (a) or (b), compounds according to the invention are compounds of formula (I) wherein Y², Y³ and Y⁴ are hydrogen atoms and Y⁵ is halogen atom.

Processes for the Preparation of the Active Ingredients

The present invention also relates to processes for the preparation of compounds of formula (I). Unless indicated otherwise, the radicals A, B, Q¹, Y¹, Y², Y³, Y⁴, Y⁵, Z, L, m, n, W and X have the meanings given above for the compounds of formula (I). These definitions apply not only to the end products of the formula (I) but likewise to all intermediates.

Compounds of formula (I) as herein-defined can be prepared by a process P1 which comprises the step of reacting a compound of formula (II) with a compound of formula (III):

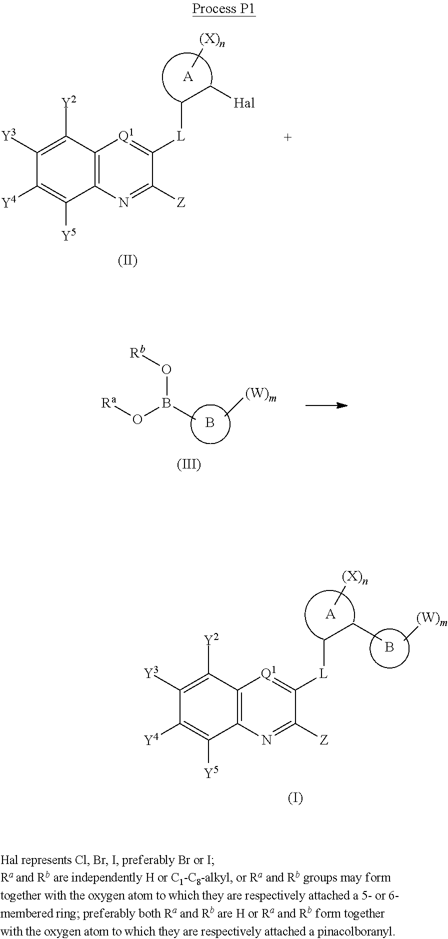

Process P1 can be performed in the presence of a transition metal catalyst such as palladium and if appropriate in the presence of a phosphine ligand or a N-heterocyclic carbene ligand, if appropriate in the presence of a base and if appropriate in the presence of a solvent according to known processes.

Halogenoaryl derivatives of formula (II) can be prepared by diazotation of an aniline of formula (IV) or one of its salts according to known processes (Patai's Chemistry of Functional Groups—Amino, Nitroso, Nitro and Related Groups—1996).

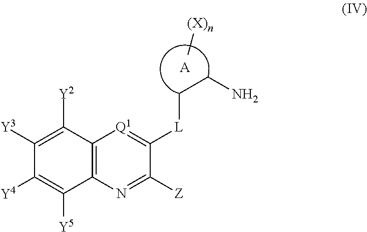

Halogenoaryl derivatives of formula (II) can also be prepared by aromatic nucleophilic substitution according to known processes (Journal of Heterocyclic Chemistry (2008), 45, 1199 and Synthetic Communications (1999), 29, 1393).

Anilines of formula (IV) can be prepared by reduction of a nitro group of formula (V) or one of its salts according to known processes (Patai's Chemistry of Functional Groups—Amino, Nitroso, Nitro and Related Groups—1996).

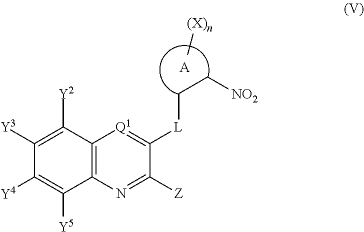

Boronic acid or boronic ester derivatives of formula (III) are commercially available or can be prepared by known processes.

Process P1 can be carried out in the presence of a catalyst, such as a metal salt or complex. Suitable metal derivatives for this purpose are transition metal catalysts such as palladium. Suitable metal salts or complexes for this purpose are for example, palladium chloride, palladium acetate, tetrakis(triphenylphosphine)palladium(0), bis(dibenzylideneacetone)palladium(0), tris(dibenzylideneacetone)dipalladium(0), bis(triphenylphosphine)palladium(II) dichloride, [1,1′-bis(diphenylphosphino)ferrocene]dichloropalladium(II), bis(cinnamyl)dichlorodipalladium(II), bis(allyl)-dichlorodipalladium(II) or [1,1′-Bis(di-tert-butylphosphino)ferrocene]dichloropalladium(II).

It is also possible to generate a palladium complex in the reaction mixture by separate addition to the reaction of a palladium salt and a ligand or salt, such as triethylphosphine, tri-tert-butylphosphine, tri-tert-butylphosphonium tetrafluoroborate, tricyclohexylphosphine, 2-(dicyclohexylphosphino)biphenyl, 2-(di-tert-butylphosphino)biphenyl, 2-(dicyclohexylphosphino)-2′-(N,N-dimethylamino)biphenyl, 2-(tert-butylphosphino)-2′-(N,N-dimethylamino)biphenyl, 2-di-tert-butylphosphino-2′,4′,6′-triisopropylbiphenyl 2-dicyclohexylphosphino-2′,4′,6′-triisopropylbiphenyl, 2-dicyclohexylphosphino-2,6′-dimethoxybiphenyl, 2-dicyclohexylphosphino-2′,6′-diisopropoxybiphenyl, triphenyl-phosphine, tris-(o-tolyl)phosphine, sodium 3-(diphenylphosphino)benzenesulfonate, tris-2-(methoxy-phenyl)phosphine, 2,2′-bis(diphenylphosphino)-1,1′-binaphthyl, 1,4-bis(diphenylphosphino)butane, 1,2-bis(diphenylphosphino) ethane, 1,4-bis(dicyclohexylphosphino)butane, 1,2-bis(dicyclohexylphosphino)-ethane, 2-(dicyclohexylphosphino)-2′-(N,N-dimethylamino)-biphenyl, 1,1′-bis(diphenylphosphino)-ferrocene, (R)-(−)-1-[(S)-2-diphenyl-phosphino)ferrocenyl]ethyldicyclohexylphosphine, tris-(2,4-tert-butyl-phenyl)phosphite, di(1-adamantyl)-2-morpholinophenylphosphine or 1,3-bis(2,4,6-trimethylphenyl)imidazolium chloride.

It is also advantageous to choose the appropriate catalyst and/or ligand from commercial catalogues such as “Metal Catalysts for Organic Synthesis” by Strem Chemicals or “Phosphorous Ligands and Compounds” by Strem Chemicals.

Suitable bases for carrying out Process P1 can be inorganic and organic bases which are customary for such reactions. Preference is given to using alkaline earth metal or alkali metal hydroxides, such as sodium hydroxide, calcium hydroxide, potassium hydroxide or other ammonium hydroxide derivatives; alkaline earth metal, alkali metal or ammonium fluorides such as potassium fluoride, caesium fluoride or tetrabutylammonium fluoride; alkaline earth metal or alkali metal carbonates, such as sodium carbonate, potassium carbonate, potassium bicarbonate, sodium bicarbonate or caesium carbonate; alkali metal or alkaline earth metal acetates, such as sodium acetate, lithium acetate, potassium acetate or calcium acetate; alkali metal or alkaline earth metal phosphate, such as tripotassium phosphate alkali; alkali metal alcoholates, such as potassium tert-butoxide or sodium tert-butoxide; tertiary amines, such as trimethylamine, triethylamine, tributylamine, N,N-dimethylaniline, N,N-dicyclohexylmethylamine, N,N-diisopropylethylamine, N-methylpiperidine, N,N-dimethylaminopyridine, diazabicyclooctane (DABCO), diazabicyclononene (DBN) or diazabicycloundecene (DBU); and also aromatic bases, such as pyridine, picolines, lutidines or collidines.

Suitable solvents for carrying out process P1 can be customary inert organic solvents. Preference is given to using optionally halogenated, aliphatic, alicyclic or aromatic hydrocarbons, such as petroleum ether, pentane, hexane, heptane, cyclohexane, methylcyclohexane, benzene, toluene, xylene or decalin; chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 2-methyltetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; ureas, such as 1,3-dimethyl-3,4,5,6-tetrahydro-2(1H)-pyrimidinone; esters, such as methyl acetate or ethyl acetate, sulfoxides, such as dimethyl sulfoxide, or sulfones, such as sulfolane; and a mixture thereof.

It can also be advantageous to carry out process P1 with a co-solvent such as water or an alcohol such as methanol, ethanol, propanol, isopropanol or tert-butanol.

Process P1 may be performed in an inert atmosphere such as argon or nitrogen atmosphere. When carrying out process P1, 1 mole or an excess of compound of formula (III) and from 1 to 5 moles of base and from 0.01 to 20 mole percent of a palladium complex can be employed per mole of compound of formula (II). It is also possible to employ the reaction components in other ratios. Work-up is carried out by known methods.

Compounds of formula (I) as herein-defined can be prepared by a process P2 which comprises the step of reacting a compound of formula (VI) with a compound of formula (VII):

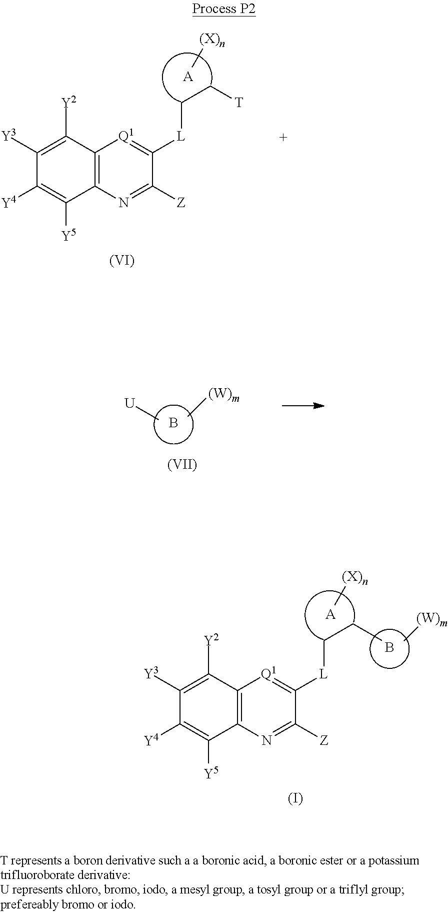

Process P2 can be performed in the presence of a transition metal catalyst such as palladium and if appropriate in the presence of a phosphine ligand or a N-heterocyclic carbene ligand, if appropriate in the presence of a base and if appropriate in the presence of a solvent according to known processes.

Boronic acid or boronic ester derivatives of formula (VI) can be prepared from halogenoaryl derivatives (III) using a reagent such as bis(pinacolato)diboron in presence of a transition metal catalyst such as palladium and if appropriate in presence of a phosphine ligand or a N-heterocyclic carbene ligand, if appropriate in presence of a base and if appropriate in presence of a solvent according to known processes.

Suitable catalysts, bases and solvents for carrying out process P2 and for the synthesis of intermediates of formula (VI) can be as disclosed in connection with process P1.

Process P2 may be performed in an inert atmosphere such as argon or nitrogen atmosphere. When carrying out process P2, 1 mole or an excess of compound of formula (VII) and from 1 to 5 moles of base and from 0.01 to 20 mole percent of a palladium complex can be employed per mole of compound of formula (VI). It is also possible to employ the reaction components in other ratios. Work-up is carried out by known methods.

Alternatively, boronic acid or boronic ester derivatives of formula (VI) can be prepared from halogenoaryl derivatives (III) by halogen metal exchange using the appropriate organometallic reagent such as n-butyllithium and the appropriate boron derivative such as trimethyl borate in the appropriate organic solvent such as an ether, preferably tetrahydrofuran or diethyether.

Halide derivatives of formula (VII) are commercially available or can be prepared by processes known by the person skilled in the art.

Compounds of formula (Ia) as herein-defined, i.e. compounds of formula (I) wherein L is NH, can be prepared by a process P3 which comprises the step of reacting a compound of formula (VIII) with a compound of formula (IX):

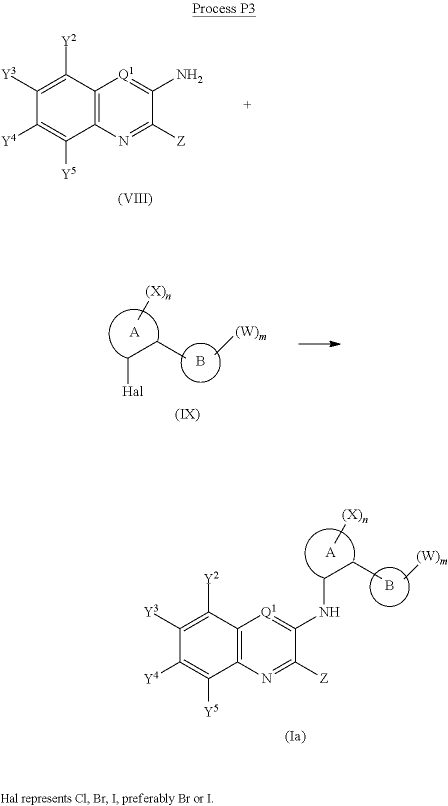

Process P3 can be performed in the presence of a transition metal catalyst such as palladium and if appropriate in the presence of a phosphine ligand or a N-heterocyclic carbene ligand, if appropriate in the presence of a base and if appropriate in the presence of a solvent according to known processes.

Amines of formula (VIII) and halogenoaryl of formula (IX) are commercially available or can be made according to methods known by the person skilled in the art.

Suitable catalysts, bases and solvents for carrying out process P3 can be as disclosed in connection with process P1.

Process P3 may be performed in an inert atmosphere such as argon or nitrogen atmosphere. When carrying out process P3, 1 mole or an excess of compound of formula (VIII) and from 1 to 5 moles of base and from 0.01 to 20 mole percent of a palladium complex can be employed per mole of compound of formula (IX). It is also possible to employ the reaction components in other ratios. Work-up is carried out by known methods.

Alternatively, compounds of formula (Ia) as herein-defined can be prepared by a process P4 which comprises the step of reacting a compound of formula (X) with a compound of formula (XI):

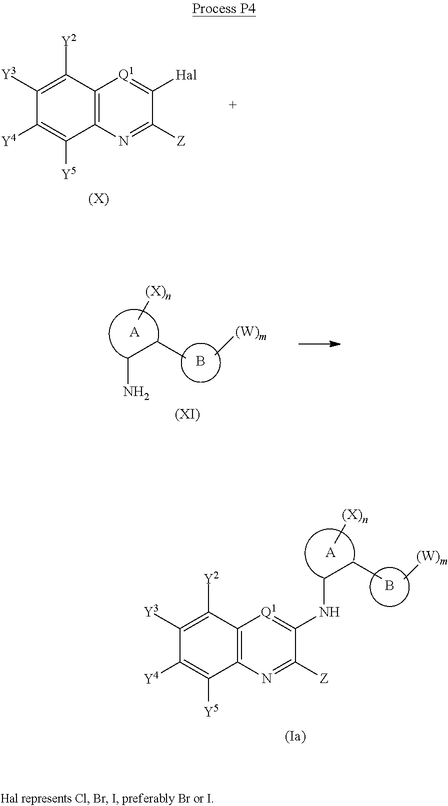

Process P4 can be performed in the presence of a transition metal catalyst such as palladium and if appropriate in the presence of a phosphine ligand or a N-heterocyclic carbene ligand, if appropriate in the presence of a base and if appropriate in the presence of a solvent according to known processes.

Halogenoaryl of formula (X) and amines of formula (XI) are commercially available or can be made according to methods known by the person skilled in the art.

Suitable catalysts, bases and solvents for carrying out process P4 can be as disclosed in connection with process P1.

Process P4 may be performed in an inert atmosphere such as argon or nitrogen atmosphere. When carrying out process P4, 1 mole or an excess of compound of formula (X) and from 1 to 5 moles of base and from 0.01 to 20 mole percent of a palladium complex can be employed per mole of compound of formula (XI). It is also possible to employ the reaction components in other ratios. Work-up is carried out by known methods.

Compounds of formula (Ib) as herein-defined, i.e. compounds of formula (I) wherein L is CH₂, can be prepared by a process P5 which comprises the step of reacting a compound of formula (XII) with a compound of formula (XIII):

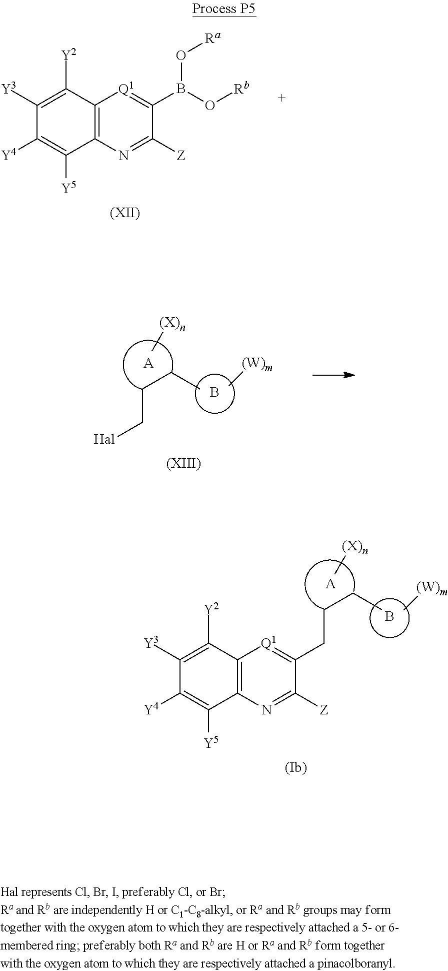

Process P5 can be performed in the presence of a transition metal catalyst such as palladium and if appropriate in the presence of a phosphine ligand or a N-heterocyclic carbene ligand, if appropriate in the presence of a base and if appropriate in the presence of a solvent according to known processes.

Boron derivatives of formula (XII) are commercially available or can be made according to methods known by the person skilled in the art.

Halides of formula (XIII), can be prepared by halogenation of alcohols of formula (XIV) according to known processes.

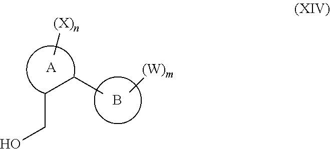

Suitable catalysts, bases and solvents for carrying out process P5 can be as disclosed in connection with process P1.

Process P5 may be performed in an inert atmosphere such as argon or nitrogen atmosphere. When carrying out process P5, 1 mole or an excess of compound of formula (XII) and from 1 to 5 moles of base and from 0.01 to 20 mole percent of a palladium complex can be employed per mole of compound of formula (XIII). It is also possible to employ the reaction components in other ratios. Work-up is carried out by known methods.

Alternatively, compounds of formula (Ib) as herein-defined can be prepared by a process P6 which comprises the step of reacting a compound of formula (XV) with a compound of formula (XVI):

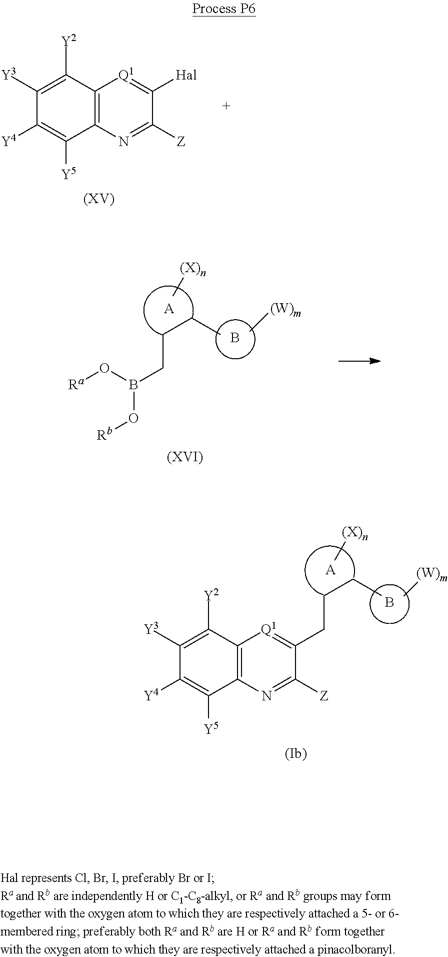

Process P6 can be performed in the presence of a transition metal catalyst such as palladium and if appropriate in the presence of a phosphine ligand or a N-heterocyclic carbene ligand, if appropriate in the presence of a base and if appropriate in the presence of a solvent according to known processes.

Halides of formula (XV) and boron derivatives of formula (XVI) are commercially available or can be made according to methods known by the person skilled in the art.

Suitable catalysts, bases and solvents for carrying out process P6 can be as disclosed in connection with process P1.

Process P6 may be performed in an inert atmosphere such as argon or nitrogen atmosphere. When carrying out process P6, 1 mole or an excess of compound of formula (XV) and from 1 to 5 moles of base and from 0.01 to 20 mole percent of a palladium complex can be employed per mole of compound of formula (XVI). It is also possible to employ the reaction components in other ratios. Work-up is carried out by known methods.

Compounds of formula (Ia) may be used for the preparation of compounds (Id) (i.e. compound of formula (I) wherein L is NR³ wherein R³ is a C₁-C₈-alkyl) according to process P7:

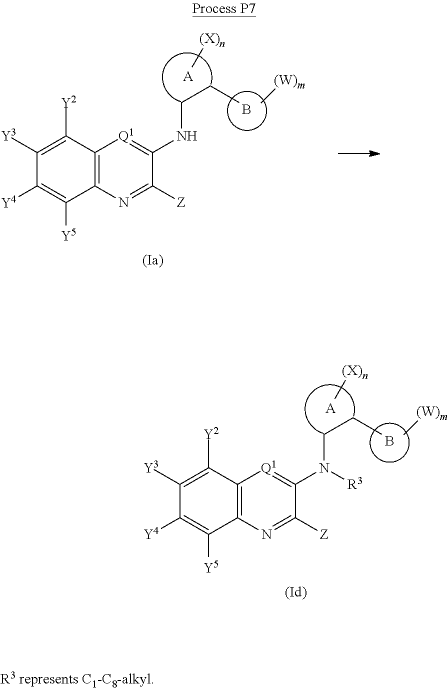

Compounds of formula (Ia) made according to processes P1, P2, P3 or P4 can be used to make compounds of formula (Id). Typically, compounds of formula (Ia) are treated with a base such as sodium hydride and an alkyl halide, preferentially an iodoalkyl such as iodomethane. The reaction is usually carried out in polar aprotic solvents such as dimethylformamide.

When carrying out process P7, 1 mole or an excess of compound of formula (Ia) and from 1 to 5 moles of base can be employed per mole of compound of alkyl halide. It is also possible to employ the reaction components in other ratios. Work-up is carried out by known methods.

Processes P1, P2, P3, P4, P5, P6 and P7 are generally carried out under atmospheric pressure. It is also possible to operate under elevated or reduced pressure.

When carrying out processes P1, P2, P3, P4, P5, P6 and P7, the reaction temperatures can be varied within a relatively wide range. In general, these processes are carried out at temperatures from −78° C. to 200° C., preferably from −78° C. to 150° C. A way to control the temperature for the processes is to use microwave technology.

In general, the reaction mixture is concentrated under reduced pressure. The residue that remains can be freed by known methods, such as chromatography or crystallization, from any impurities that can still be present.

Work-up is carried out by customary methods. Generally, the reaction mixture is treated with water and the organic phase is separated off and, after drying, concentrated under reduced pressure. If appropriate, the remaining residue can, be freed by customary methods, such as chromatography, crystallization or distillation, from any impurities that may still be present.

The compounds of formula (I) can be prepared according to the general processes of preparation described above. It will nevertheless be understood that, on the basis of his general knowledge and of available publications, the skilled worker will be able to adapt the methods according to the specifics of each compound, which it is desired to synthesize.

### Compositions and Formulations

The present invention further relates to a composition, in particular a composition for controlling unwanted microorganisms. The compositions may be applied to the microorganisms and/or in their habitat.

The composition typically comprises at least one compound of formula (I) and at least one agriculturally suitable auxiliary, e.g. carrier(s) and/or surfactant(s).

A carrier is a solid or liquid, natural or synthetic, organic or inorganic substance that is generally inert. The carrier generally improves the application of the compounds, for instance, to plants, plants parts or seeds. Examples of suitable solid carriers include, but are not limited to, ammonium salts, natural rock flours, such as kaolins, clays, talc, chalk, quartz, attapulgite, montmorillonite and diatomaceous earth, and synthetic rock flours, such as finely divided silica, alumina and silicates. Examples of typically useful solid carriers for preparing granules include, but are not limited to crushed and fractionated natural rocks such as calcite, marble, pumice, sepiolite and dolomite, synthetic granules of inorganic and organic flours and granules of organic material such as paper, sawdust, coconut shells, maize cobs and tobacco stalks. Examples of suitable liquid carriers include, but are not limited to, water, organic solvents and combinations thereof. Examples of suitable solvents include polar and nonpolar organic chemical liquids, for example from the classes of aromatic and nonaromatic hydrocarbons (such as cyclohexane, paraffins, alkylbenzenes, xylene, toluene alkylnaphthalenes, chlorinated aromatics or chlorinated aliphatic hydrocarbons such as chlorobenzenes, chloroethylenes or methylene chloride), alcohols and polyols (which may optionally also be substituted, etherified and/or esterified, such as butanol or glycol), ketones (such as acetone, methyl ethyl ketone, methyl isobutyl ketone or cyclohexanone), esters (including fats and oils) and (poly)ethers, unsubstituted and substituted amines, amides (such as dimethylformamide), lactams (such as N-alkylpyrrolidones) and lactones, sulfones and sulfoxides (such as dimethyl sulfoxide). The carrier may also be a liquefied gaseous extender, i.e. liquid which is gaseous at standard temperature and under standard pressure, for example aerosol propellants such as halohydrocarbons, butane, propane, nitrogen and carbon dioxide. The amount of carrier typically ranges from 1 to 99.99%, preferably from 5 to 99.9%, more preferably from 10 to 99.5%, and most preferably from 20 to 99% by weight of the composition.

The surfactant can be an ionic (cationic or anionic) or non-ionic surfactant, such as ionic or non-ionic emulsifier(s), foam former(s), dispersant(s), wetting agent(s) and any mixtures thereof. Examples of suitable surfactants include, but are not limited to, salts of polyacrylic acid, salts of lignosulfonic acid, salts of phenolsulfonic acid or naphthalenesulfonic acid, polycondensates of ethylene and/or propylene oxide with fatty alcohols, fatty acids or fatty amines (polyoxyethylene fatty acid esters, polyoxyethylene fatty alcohol ethers, for example alkylaryl polyglycol ethers), substituted phenols (preferably alkylphenols or arylphenols), salts of sulfosuccinic esters, taurine derivatives (preferably alkyl taurates), phosphoric esters of polyethoxylated alcohols or phenols, fatty esters of polyols and derivatives of compounds containing sulfates, sulfonates, phosphates (for example, alkylsulfonates, alkyl sulfates, arylsulfonates) and protein hydrolysates, lignosulfite waste liquors and methylcellulose. A surfactant is typically used when the compound of formula (I) and/or the carrier is insoluble in water and the application is made with water. Then, the amount of surfactants typically ranges from 5 to 40% by weight of the composition.

Further examples of suitable auxiliaries include water repellents, siccatives, binders (adhesive, tackifier, fixing agent, such as carboxymethylcellulose, natural and synthetic polymers in the form of powders, granules or latices, such as gum arabic, polyvinyl alcohol and polyvinyl acetate, natural phospholipids such as cephalins and lecithins and synthetic phospholipids, polyvinylpyrrolidone and tylose), thickeners, stabilizers (e.g. cold stabilizers, preservatives, antioxidants, light stabilizers, or other agents which improve chemical and/or physical stability), dyes or pigments (such as inorganic pigments, e.g. iron oxide, titanium oxide and Prussian Blue; organic dyes, e.g. alizarin, azo and metal phthalocyanine dyes), antifoams (e.g. silicone antifoams and magnesium stearate), preservatives (e.g. dichlorophene and benzyl alcohol hemiformal), secondary thickeners (cellulose derivatives, acrylic acid derivatives, xanthan, modified clays and finely divided silica), stickers, gibberellins and processing auxiliaries, mineral and vegetable oils, perfumes, waxes, nutrients (including trace nutrients, such as salts of iron, manganese, boron, copper, cobalt, molybdenum and zinc), protective colloids, thixotropic substances, penetrants, sequestering agents and complex formers.

The choice of the auxiliaries is related to the intended mode of application of the compound of formula (I) and/or on the physical properties. Furthermore, the auxiliaries may be chosen to impart particular properties (technical, physical and/or biological properties) to the compositions or use forms prepared therefrom. The choice of auxiliaries may allow customizing the compositions to specific needs.

The composition may be in any customary form, such as solutions (e.g. aqueous solutions), emulsions, wettable powders, water- and oil-based suspensions, powders, dusts, pastes, soluble powders, soluble granules, granules for broadcasting, suspoemulsion concentrates, natural or synthetic products impregnated with the compound of formula (I), fertilizers and also microencapsulations in polymeric substances. The compound of formula (I) may be present in a suspended, emulsified or dissolved form.

The composition may be provided to the end user as ready-for-use formulation, i.e. the compositions may be directly applied to the plants or seeds by a suitable device, such as a spraying or dusting device. Alternatively, the compositions may be provided to the end user in the form of concentrates which have to be diluted, preferably with water, prior to use.

The composition can be prepared in conventional manners, for example by mixing the compound of formula (I) with one or more suitable auxiliaries, such as disclosed herein above.

The composition contains generally from 0.01 to 99% by weight, from 0.05 to 98% by weight, preferably from 0.1 to 95% by weight, more preferably from 0.5 to 90% by weight, most preferably from 1 to 80% by weight of the compound of formula (I). It is possible that a composition comprises two or more compounds formula (I). In such case the outlined ranges refer to the total amount of compounds of the present invention.

Mixtures/Combinations

The compound of formula (I) and composition comprising thereof can be mixed with other active ingredients like fungicides, bactericides, acaricides, nematicides, insecticides, herbicides, fertilizers, growth regulators, safeners or semiochemicals. This may allow to broaden the activity spectrum or to prevent development of resistance. Examples of known fungicides, insecticides, acaricides, nematicides and bactericides are disclosed in the Pesticide Manual, 17th Edition.

Examples of especially preferred fungicides which could be mixed with the compound of formula (I) and the composition are:

1) Inhibitors of the ergosterol biosynthesis, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) Pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1 S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) Mefentrifluconazole, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)-phenyl]sulfanyl}-2,5-dimethylphenyl)-N-ethyl-N-methylimidoformamide, (1.073) N′-(4-{3-[(difluoro-methyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) Ipfentrifluconazole.
2) Inhibitors of the respiratory chain at complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) Isofetamid, (2.010) isopyrazam (anti-epimeric enantiomer 1R,4S,9S), (2.011) isopyrazam (anti-epimeric enantiomer 1 S,4R,9R), (2.012) isopyrazam (anti-epimeric racemate 1RS,4SR,9SR), (2.013) isopyrazam (mixture of syn-epimeric racemate 1RS,4SR,9RS and anti-epimeric racemate 1RS,4SR,9SR), (2.014) isopyrazam (syn-epimeric enantiomer 1R,4S,9R), (2.015) isopyrazam (syn-epimeric enantiomer 1 S,4R,9S), (2.016) isopyrazam (syn-epimeric racemate 1 RS,4SR,9RS), (2.017) penflufen, (2.018) penthiopyrad, (2.019) pydiflumetofen, (2.020) Pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) Fluindapyr, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034)N-(2-cyclopentyl-5-fluorobenzyl)-N-cyclopropyl-3-(difluoromethyl)-5-fluoro-1-methyl-1H-pyrazole-4-carboxamide, (2.035)N-(2-tert-butyl-5-methylbenzyl)-N-cyclopropyl-3-(difluoromethyl)-5-fluoro-1-methyl-1H-pyrazole-4-carboxamide, (2.036)N-(2-tert-butylbenzyl)-N-cyclopropyl-3-(difluoromethyl)-5-fluoro-1-methyl-1H-pyrazole-4-carboxamide, (2.037)N-(5-chloro-2-ethylbenzyl)-N-cyclopropyl-3-(difluoromethyl)-5-fluoro-1-methyl-1H-pyrazole-4-carboxamide, (2.038) isoflucypram, (2.039)N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040)N-[(1S,4R)-9-(dichloromethylene)-1,2,3,4-tetrahydro-1,4-methanonaphthalen-5-yl]-3-(difluoro-methyl)-1-methyl-1H-pyrazole-4-carboxamide, (2.041)N-[1-(2,4-dichlorophenyl)-1-methoxypropan-2-yl]-3-(difluoromethyl)-1-methyl-1H-pyrazole-4-carboxamide, (2.042)N-[2-chloro-6-(trifluoromethyl)benzyl]-N-cyclopropyl-3-(difluoromethyl)-5-fluoro-1-methyl-1H-pyrazole-4-carboxamide, (2.043)N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044)N-[5-chloro-2-(trifluoromethyl)benzyl]-N-cyclopropyl-3-(difluoromethyl)-5-fluoro-1-methyl-1H-pyrazole-4-carboxamide, (2.045)N-cyclopropyl-3-(difluoromethyl)-5-fluoro-1-methyl-N-[5-methyl-2-(trifluoromethyl)benzyl]-1H-pyrazole-4-carboxamide, (2.046)N-cyclopropyl-3-(difluoromethyl)-5-fluoro-N-(2-fluoro-6-isopropylbenzyl)-1-methyl-1H-pyrazole-4-carboxamide, (2.047)N-cyclopropyl-3-(difluoromethyl)-5-fluoro-N-(2-isopropyl-5-methylbenzyl)-1-methyl-1H-pyrazole-4-carboxamide, (2.048)N-cyclopropyl-3-(difluoromethyl)-5-fluoro-N-(2-isopropylbenzyl)-1-methyl-1H-pyrazole-4-carbothioamide, (2.049)N-cyclopropyl-3-(difluoromethyl)-5-fluoro-N-(2-isopropylbenzyl)-1-methyl-1H-pyrazole-4-carboxamide, (2.050)N-cyclopropyl-3-(difluoromethyl)-5-fluoro-N-(5-fluoro-2-isopropylbenzyl)-1-methyl-1H-pyrazole-4-carboxamide, (2.051)N-cyclopropyl-3-(difluoromethyl)-N-(2-ethyl-4,5-dimethylbenzyl)-5-fluoro-1-methyl-1H-pyrazole-4-carboxamide, (2.052)N-cyclopropyl-3-(difluoromethyl)-N-(2-ethyl-5-fluorobenzyl)-5-fluoro-1-methyl-1H-pyrazole-4-carboxamide, (2.053)N-cyclopropyl-3-(difluoromethyl)-N-(2-ethyl-5-methylbenzyl)-5-fluoro-1-methyl-1H-pyrazole-4-carboxamide, (2.054)N-cyclopropyl-N-(2-cyclopropyl-5-fluorobenzyl)-3-(difluoromethyl)-5-fluoro-1-methyl-1H-pyrazole-4-carboxamide, (2.055)N-cyclopropyl-N-(2-cyclopropyl-5-methylbenzyl)-3-(difluoromethyl)-5-fluoro-1-methyl-1H-pyrazole-4-carboxamide, (2.056)N-cyclopropyl-N-(2-cyclopropylbenzyl)-3-(difluoromethyl)-5-fluoro-1-methyl-1H-pyrazole-4-carboxamide, (2.057) pyrapropoyne.
3) Inhibitors of the respiratory chain at complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) mandestrobin, (3.027)N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate, (3.030) metyltetraprole, (3.031) florylpicoxamid.
4) Inhibitors of the mitosis and cell division, for example (4.001) carbendazim, (4.002) diethofencarb, (4.003) ethaboxam, (4.004) fluopicolide, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023)N-(2-bromo-6-fluorophenyl)-4-(2-chloro-4-fluorophenyl)-1,3-dimethyl-1H-pyrazol-5-amine, (4.024)N-(2-bromophenyl)-4-(2-chloro-4-fluorophenyl)-1,3-dimethyl-1H-pyrazol-5-amine, (4.025)N-(4-chloro-2,6-difluorophenyl)-4-(2-chloro-4-fluorophenyl)-1,3-dimethyl-1H-pyrazol-5-amine.
5) Compounds capable to have a multisite action, for example (5.001) bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) metiram zinc, (5.017) oxine-copper, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.
6) Compounds capable to induce a host defence, for example (6.001) acibenzolar-S-methyl, (6.002) isotianil, (6.003) probenazole, (6.004) tiadinil.
7) Inhibitors of the amino acid and/or protein biosynthesis, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.
8) Inhibitors of the ATP production, for example (8.001) silthiofam.
9) Inhibitors of the cell wall synthesis, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.
10) Inhibitors of the lipid and membrane synthesis, for example (10.001) propamocarb, (10.002) propamocarb hydrochloride, (10.003) tolclofos-methyl.
11) Inhibitors of the melanin biosynthesis, for example (11.001) tricyclazole, (11.002) 2,2,2-trifluoroethyl {3-methyl-1-[(4-methylbenzoyl)amino]butan-2-yl}carbamate.
12) Inhibitors of the nucleic acid synthesis, for example (12.001) benalaxyl, (12.002) benalaxyl-M (kiralaxyl), (12.003) metalaxyl, (12.004) metalaxyl-M (mefenoxam).
13) Inhibitors of the signal transduction, for example (13.001) fludioxonil, (13.002) iprodione, (13.003) procymidone, (13.004) proquinazid, (13.005) quinoxyfen, (13.006) vinclozolin.
14) Compounds capable to act as an uncoupler, for example (14.001) fluazinam, (14.002) meptyldinocap.
15) Further compounds, for example (15.001) Abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) oxathiapiprolin, (15.023) oxyfenthiin, (15.024) pentachlorophenol and salts, (15.025) phosphorous acid and its salts, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) dipymetitrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)-piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) Ipflufenoquin, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts, (15.046) 3-(4,4,5-trifluoro-3,3-dimethyl-3,4-dihydroisoquinolin-1-yl)quinoline, (15.047) quinofumelin, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butanoic acid, (15.050) 5-amino-1,3,4-thiadiazole-2-thiol, (15.051) 5-chloro-N′-phenyl-N′-(prop-2-yn-1-yl)thiophene-2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, (15.062) 5-fluoro-4-imino-3-methyl-1-[(4-methylphenyl)sulfonyl]-3,4-dihydropyrimidin-2(1H)-one, (15.063) aminopyrifen.

All named mixing partners of the classes (1) to (15) as described here above can be present in the form of the free compound and/or, if their functional groups enable this, an agriculturally acceptable salt thereof.

The compound of formula (I) and the composition comprising thereof may also be combined with one or more biological control agents.

Examples of biological control agents which may be combined with the compound of formula (I) and composition comprising thereof are:

(A) Antibacterial agents selected from the group of:
(A1) bacteria, such as (A1.1) Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051); (A1.2) Bacillus amyloliquefaciens, in particular strain D747 (available as Double Nickel™ from Certis, US, having accession number FERM BP-8234 and disclosed in U.S. Pat. No. 7,094,592); (A1.3) Bacillus pumilus, in particular strain BU F-33 (having NRRL Accession No. 50185); (A1.4) Bacillus subtilis var. amyloliquefaciens strain FZB24 (available as Taegro® from Novozymes, US); (A1.5) a Paenibacillus sp. strain having Accession No. NRRL B-50972 or Accession No. NRRL B-67129 and described in International Patent Publication No. WO 2016/154297; and
(A2) fungi, such as (A2.1) Aureobasidium pullulans, in particular blastospores of strain DSM14940; (A2.2) Aureobasidium pullulans blastospores of strain DSM 14941; (A2.3) Aureobasidium pullulans, in particular mixtures of blastospores of strains DSM14940 and DSM14941;
(B) Fungicides selected from the group of:
(B1) bacteria, for example (B1.1) Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051); (B11.2) Bacillus pumilus, in particular strain QST2808 (available as SONATA® from Bayer CropScience LP, US, having Accession No. NRRL B-30087 and described in U.S. Pat. No. 6,245,551); (B11.3) Bacillus pumilus, in particular strain GB34 (available as Yield Shield® from Bayer AG, DE); (B11.4) Bacillus pumilus, in particular strain BU F-33 (having NRRL Accession No. 50185); (B11.5) Bacillus amyloliquefaciens, in particular strain D747 (available as Double Nickel™ from Certis, US, having accession number FERM BP-8234 and disclosed in U.S. Pat. No. 7,094,592); (B1.6) Bacillus subtilis Y1336 (available as BIOBAC® WP from Bion-Tech, Taiwan, registered as a biological fungicide in Taiwan under Registration Nos. 4764, 5454, 5096 and 5277); (B11.7) Bacillus amyloliquefaciens strain MBI 600 (available as SUBTILEX from BASF SE); (B11.8) Bacillus subtilis strain GB03 (available as Kodiak® from Bayer AG, DE); (B11.9) Bacillus subtilis var. amyloliquefaciens strain FZB24 (available from Novozymes Biologicals Inc., Salem, Va. or Syngenta Crop Protection, LLC, Greensboro, N.C. as the fungicide TAEGRO® or TAEGRO® ECO (EPA Registration No. 70127-5); (B11.10) Bacillus mycoides, isolate J (available as BmJ TGAI or WG from Certis USA); (B1.11) Bacillus licheniformis, in particular strain SB3086 (available as EcoGuard™ Biofungicide and Green Releaf from Novozymes); (B1.12) a Paenibacillus sp. strain having Accession No. NRRL B-50972 or Accession No. NRRL B-67129 and described in International Patent Publication No. WO 2016/154297.

In some embodiments, the biological control agent is a Bacillus subtilis or Bacillus amyloliquefaciens strain that produces a fengycin or plipastatin-type compound, an iturin-type compound, and/or a surfactin-type compound. For background, see the following review article: Ongena, M., et al., “Bacillus Lipopeptides: Versatile Weapons for Plant Disease Biocontrol,” Trends in Microbiology, Vol 16, No. 3, March 2008, pp. 115-125. Bacillus strains capable of producing lipopeptides include Bacillus subtilis QST713 (available as SERENADE OPTI or SERENADE ASO from Bayer CropScience LP, US, having NRRL Accession No. B21661 and described in U.S. Pat. No. 6,060,051), Bacillus amyloliquefaciens strain D747 (available as Double Nickel™ from Certis, US, having accession number FERM BP-8234 and disclosed in U.S. Pat. No. 7,094,592); Bacillus subtilis MBI600 (available as SUBTILEX® from Becker Underwood, US EPA Reg. No. 71840-8); Bacillus subtilis Y1336 (available as BIOBAC® WP from Bion-Tech, Taiwan, registered as a biological fungicide in Taiwan under Registration Nos. 4764, 5454, 5096 and 5277); Bacillus amyloliquefaciens, in particular strain FZB42 (available as RHIZOVITAL® from ABiTEP, DE); and Bacillus subtilis var. amyloliquefaciens FZB24 (available from Novozymes Biologicals Inc., Salem, Va. or Syngenta Crop Protection, LLC, Greensboro, N.C. as the fungicide TAEGRO® or TAEGRO® ECO (EPA Registration No. 70127-5); and

(B2) fungi, for example: (B2.1) Coniothyrium minitans, in particular strain CON/M/91-8 (Accession No. DSM-9660; e.g. Contans® from Bayer); (B2.2) Metschnikowia fructicola, in particular strain NRRL Y-30752 (e.g. Shemer®); (B2.3) Microsphaeropsis ochracea (e.g. Microx® from Prophyta); (B2.5) Trichoderma spp., including Trichoderma atroviride, strain SC1 described in International Application No. PCT/IT2008/000196); (B2.6) Trichoderma harzianum rifai strain KRL-AG2 (also known as strain T-22, /ATCC 208479, e.g. PLANTSHIELD T-22G, Rootshield®, and TurfShield from BioWorks, US); (B2.14) Gliocladium roseum, strain 321U from W. F. Stoneman Company LLC; (B2.35) Talaromyces flavus, strain V117b; (B2.36) Trichoderma asperellum, strain ICC 012 from Isagro; (B2.37) Trichoderma asperellum, strain SKT-1 (e.g. ECO-HOPE® from Kumiai Chemical Industry); (B2.38) Trichoderma atroviride, strain CNCM-1237 (e.g. Esquive® WP from Agrauxine, FR); (B2.39) Trichoderma atroviride, strain no. V08/002387; (B2.40) Trichoderma atroviride, strain NMI no. V08/002388; (B2.41) Trichoderma atroviride, strain NMI no. V08/002389; (B2.42) Trichoderma atroviride, strain NMI no. V08/002390; (B2.43) Trichoderma atroviride, strain LC52 (e.g. Tenet by Agrimm Technologies Limited); (B2.44) Trichoderma atroviride, strain ATCC 20476 (IMI 206040); (B2.45) Trichoderma atroviride, strain T11 (IMI352941/CECT20498); (B2.46) Trichoderma harmatum; (B2.47) Trichoderma harzianum; (B2.48) Trichoderma harzianum rifai T39 (e.g. Trichodex® from Makhteshim, US); (B2.49) Trichoderma harzianum, in particular, strain KD (e.g. Trichoplus from Biological Control Products, SA (acquired by Becker Underwood)); (B2.50) Trichoderma harzianum, strain ITEM 908 (e.g. Trianum-P from Koppert); (B2.51) Trichoderma harzianum, strain TH35 (e.g. Root-Pro by Mycontrol); (B2.52) Trichoderma virens (also known as Gliocladium virens), in particular strain GL-21 (e.g. SoilGard 12G by Certis, US); (B2.53) Trichoderma viride, strain TV1(e.g. Trianum-P by Koppert); (B2.54) Ampelomyces quisqualis, in particular strain AQ 10 (e.g. AQ 10® by IntrachemBio Italia); (B2.56) Aureobasidium pullulans, in particular blastospores of strain DSM14940; (B2.57) Aureobasidium pullulans, in particular blastospores of strain DSM 14941; (B2.58) Aureobasidium pullulans, in particular mixtures of blastospores of strains DSM14940 and DSM 14941 (e.g. Botector® by bio-ferm, CH); (B2.64) Cladosporium cladosporioides, strain H39 (by Stichting Dienst Landbouwkundig Onderzoek); (B2.69) Gliocladium catenulatum (Synonym: Clonostachys rosea f. catenulate) strain J1446 (e.g. Prestop® by AgBio Inc. and also e.g. Primastop® by Kemira Agro Oy); (B2.70) Lecanicillium lecanii (formerly known as Verticillium lecanii) conidia of strain KV01 (e.g. Vertalec® by Koppert/Arysta); (B2.71) Penicillium vermiculatum; (B2.72) Pichia anomala, strain WRL-076 (NRRL Y-30842); (B2.75) Trichoderma atroviride, strain SKT-1 (FERM P-16510); (B2.76) Trichoderma atroviride, strain SKT-2 (FERM P-16511); (B2.77) Trichoderma atroviride, strain SKT-3 (FERM P-17021); (B2.78) Trichoderma gamsii (formerly T. viride), strain ICCO80 (IMI CC 392151 CABI, e.g. BioDerma by AGROBIOSOL DE MEXICO, S.A. DE C.V.); (B2.79) Trichoderma harzianum, strain DB 103 (e.g., T-Gro 7456 by Dagutat Biolab); (B2.80) Trichoderma polysporum, strain IMI 206039 (e.g. Binab TF WP by BINAB Bio-Innovation AB, Sweden); (B2.81) Trichoderma stromaticum (e.g. Tricovab by Ceplac, Brazil); (B2.83) Ulocladium oudemansii, in particular strain HRU3 (e.g. Botry-Zen® by Botry-Zen Ltd, NZ); (B2.84) Verticillium albo-atrum (formerly V. dahliae), strain WCS850 (CBS 276.92; e.g. Dutch Trig by Tree Care Innovations); (B2.86) Verticillium chlamydosporium; (B2.87) mixtures of Trichoderma asperellum strain ICC 012 and Trichoderma gamsii strain ICC 080 (product known as e.g. BIO-TAM™ from Bayer CropScience LP, US).

Further examples of biological control agents which may be combined with the compound of formula (I) and composition comprising thereof are:

bacteria selected from the group consisting of Bacillus cereus, in particular B. cereus strain CNCM 1-1562 and Bacillus firmus, strain 1-1582 (Accession number CNCM I-1582), Bacillus subtilis strain OST 30002 (Accession No. NRRL B-50421), Bacillus thuringiensis, in particular B. thuringiensis subspecies israelensis (serotype H-14), strain AM65-52 (Accession No. ATCC 1276), B. thuringiensis subsp. aizawai, in particular strain ABTS-1857 (SD-1372), B. thuringiensis subsp. kurstaki strain HD-1, B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), and Streptomyces galbus strain AQ 6047 (Acession Number NRRL 30232);
fungi and yeasts selected from the group consisting of Beauveria bassiana, in particular strain ATCC 74040, Lecanicillium spp., in particular strain HRO LEC 12, Metarhizium anisopliae, in particular strain F52 (DSM3884 or ATCC 90448), Paecilomyces fumosoroseus (now: Isaria fumosorosea), in particular strain IFPC 200613, or strain Apopka 97 (Accesion No. ATCC 20874), and Paecilomyces lilacinus, in particular P. lilacinus strain 251 (AGAL 89/030550);
viruses selected from the group consisting of Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, and Spodoptera littoralis (African cotton leafworm) NPV.
bacteria and fungi which can be added as ‘inoculant’ to plants or plant parts or plant organs and which, by virtue of their particular properties, promote plant growth and plant health. Examples are: Agrobacterium spp., Azorhizobium caulinodans, Azospirillum spp., Azotobacter spp., Bradyrhizobium spp., Burkholderia spp., in particular Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., in particular Rhizobium trifolii, Rhizopogon spp., Scleroderma spp., Suillus spp., and Streptomyces spp.
plant extracts and products formed by microorganisms including proteins and secondary metabolites which can be used as biological control agents, such as Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), Pyrethrum/Pyrethrins, Quassia amara, Quercus, Quillaja, Regalia, “Requiem™ Insecticide”, rotenone, Ryania/ryanodine, Symphytum officinale, Tanacetum vulgare, thymol, Triact 70, TriCon, Tropaeulum majus, Urtica dioica, Veratrin, Viscum album, Brassicaceae extract, in particular oilseed rape powder or mustard powder.

Examples of insecticides, acaricides and nematicides, respectively, which could be mixed with the compound of formula (I) and composition comprising thereof are:

(1) Acetylcholinesterase (AChE) inhibitors, such as, for example, carbamates, for example alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel blockers, such as, for example, cyclodiene-organochlorines, for example chlordane and endosulfan or phenylpyrazoles (fiproles), for example ethiprole and fipronil.
(3) Sodium channel modulators, such as, for example, pyrethroids, e.g. acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomer], deltamethrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans-isomer], prallethrin, pyrethrins (pyrethrum), resmethrin, silafluofen, tefluthrin, tetramethrin, tetramethrin [(1R)-isomer)], tralomethrin and transfluthrin or DDT or methoxychlor.
(4) Nicotinic acetylcholine receptor (nAChR) competitive modulators, such as, for example, neonicotinoids, e.g. acetamiprid, clothianidin, dinotefuran, imidacloprid, nitenpyram, thiacloprid and thiamethoxam or nicotine or sulfoxaflor or flupyradifurone.
(5) Nicotinic acetylcholine receptor (nAChR) allosteric modulators, such as, for example, spinosyns, e.g. spinetoram and spinosad.
(6) Glutamate-gated chloride channel (GluCl) allosteric modulators, such as, for example, avermectins/milbemycins, for example abamectin, emamectin benzoate, lepimectin and milbemectin.
(7) Juvenile hormone mimics, such as, for example, juvenile hormone analogues, e.g. hydroprene, kinoprene and methoprene or fenoxycarb or pyriproxyfen.
(8) Miscellaneous non-specific (multi-site) inhibitors, such as, for example, alkyl halides, e.g. methyl bromide and other alkyl halides; or chloropicrine or sulphuryl fluoride or borax or tartar emetic or methyl isocyanate generators, e.g. diazomet and metam.
(9) Modulators of Chordotonal Organs, such as, for example pymetrozine or flonicamid.
(10) Mite growth inhibitors, such as, for example clofentezine, hexythiazox and diflovidazin or etoxazole.
(11) Microbial disruptors of the insect gut membrane, such as, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and B.t. plant proteins: Cry1Ab, Cry1Ac, Cry1Fa, Cry1A.105, Cry2Ab, Vip3A, mCry3A, Cry3Ab, Cry3Bb, Cry34Ab1/35Ab1.
(12) Inhibitors of mitochondrial ATP synthase, such as, ATP disruptors such as, for example, diafenthiuron or organotin compounds, for example azocyclotin, cyhexatin and fenbutatin oxide or propargite or tetradifon.
(13) Uncouplers of oxidative phosphorylation via disruption of the proton gradient, such as, for example, chlorfenapyr, DNOC and sulfluramid.
(14) Nicotinic acetylcholine receptor channel blockers, such as, for example, bensultap, cartap hydrochloride, thiocylam, and thiosultap-sodium.
(15) Inhibitors of chitin biosynthesis, type 0, such as, for example, bistrifluron, chlorfluazuron, diflubenzuron, flucycloxuron, flufenoxuron, hexaflumuron, lufenuron, novaluron, noviflumuron, teflubenzuron and triflumuron.
(16) Inhibitors of chitin biosynthesis, type 1, for example buprofezin.
(17) Moulting disruptor (in particular for Diptera, i.e. dipterans), such as, for example, cyromazine.
(18) Ecdysone receptor agonists, such as, for example, chromafenozide, halofenozide, methoxyfenozide and tebufenozide.
(19) Octopamine receptor agonists, such as, for example, amitraz.
(20) Mitochondrial complex III electron transport inhibitors, such as, for example, hydramethylnone or acequinocyl or fluacrypyrim.
(21) Mitochondrial complex I electron transport inhibitors, such as, for example from the group of the METI acaricides, e.g. fenazaquin, fenpyroximate, pyrimidifen, pyridaben, tebufenpyrad and tolfenpyrad or rotenone (Derris).
(22) Voltage-dependent sodium channel blockers, such as, for example indoxacarb or metaflumizone.
(23) Inhibitors of acetyl CoA carboxylase, such as, for example, tetronic and tetramic acid derivatives, e.g. spirodiclofen, spiromesifen and spirotetramat.
(24) Mitochondrial complex IV electron transport inhibitors, such as, for example, phosphines, e.g. aluminium phosphide, calcium phosphide, phosphine and zinc phosphide or cyanides, e.g. calcium cyanide, potassium cyanide and sodium cyanide.
(25) Mitochondrial complex II electron transport inhibitors, such as, for example, beta-ketonitrile derivatives, e.g. cyenopyrafen and cyflumetofen and carboxanilides, such as, for example, pyflubumide.
(28) Ryanodine receptor modulators, such as, for example, diamides, e.g. chlorantraniliprole, cyantraniliprole and flubendiamide,
further active compounds such as, for example, Afidopyropen, Afoxolaner, Azadirachtin, Benclothiaz, Benzoximate, Bifenazate, Broflanilide, Bromopropylate, Chinomethionat, Chloroprallethrin, Cryolite, Cyclaniliprole, Cycloxaprid, Cyhalodiamide, Dicloromezotiaz, Dicofol, epsilon-Metofluthrin, epsilon-Momfluthrin, Flometoquin, Fluazaindolizine, Fluensulfone, Flufenerim, Flufenoxystrobin, Flufiprole, Fluhexafon, Fluopyram, Fluralaner, Fluxametamide, Fufenozide, Guadipyr, Heptafluthrin, Imidaclothiz, Iprodione, kappa-Bifenthrin, kappa-Tefluthrin, Lotilaner, Meperfluthrin, Paichongding, Pyridalyl, Pyrifluquinazon, Pyriminostrobin, Spirobudiclofen, Tetramethylfluthrin, Tetraniliprole, Tetrachlorantraniliprole, Tigolaner, Tioxazafen, Thiofluoximate, Triflumezopyrim and iodomethane; furthermore preparations based on Bacillus firmus (1-1582, BioNeem, Votivo), and also the following compounds: 1-{2-fluoro-4-methyl-5-[(2,2,2-trifluoroethyl)sulfinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457) (CAS 637360-23-7), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethyl carbonate (known from EP2647626) (CAS 1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoro-propan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)-benzamide, 4-[5-(3,5-dichlorophenyl)-4,5-dihydro-5-(trifluoromethyl)-3-isoxazolyl]-2-methyl-N-(trans-1-oxido-3-thietanyl)-benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulfinyl]-propanamide, (+)-N-[3-chloro-1-(3-pyridinyl)-1H-pyrazol-4-yl]-N-ethyl-3-[(3,3,3-trifluoropropyl)sulfinyl]-propanamide and (−)-N-[3-chloro-1-(3-pyridinyl)-1H-pyrazol-4-yl]-N-ethyl-3-[(3,3,3-trifluoropropyl)sulfinyl]-propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide, (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-Pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl) oxy]phenoxy]propoxy]-2-methoxy-6-(trifluoromethyl)-pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]-hydrazinecarboxamide (known from CN 101715774 A) (CAS 1232543-85-9); 3-(2,2-dichloroethenyl)-2,2-dimethyl-4-(1H-benzimidazol-2-yl)phenyl-cyclopropanecarboxylic acid ester (known from CN 103524422 A) (CAS 1542271-46-4); (4aS)-7-chloro-2,5-dihydro-2-[[(methoxycarbonyl)[4-[(trifluoromethyl)thio]phenyl]amino]carbonyl]-indeno[1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-, 1-[N-[4-[1-[4-(1,1,2,2,2-pentafluoroethoxy)phenyl]-1H-1,2,4-triazol-3-yl]phenyl]carbamate]-α-L-mannopyranose (known from US 2014/0275503 A1) (CAS 1181213-14-8); 8-(2-cyclopropylmethoxy-4-trifluoromethyl-phenoxy)-3-(6-trifluoromethyl-pyridazin-3-yl)-3-aza-bicyclo[3.2.1]octane (CAS 1253850-56-4), (8-anti)-8-(2-cyclopropylmethoxy-4-trifluoromethyl-phenoxy)-3-(6-trifluoromethyl-pyridazin-3-yl)-3-aza-bicyclo[3.2.1]octane (CAS 933798-27-7), (8-syn)-8-(2-cyclopropylmethoxy-4-trifluoromethyl-phenoxy)-3-(6-trifluoromethyl-pyridazin-3-yl)-3-aza-bicyclo[3.2.1]octane (known from WO 2007040280 A1, WO 2007040282 A1) (CAS 934001-66-8), N-[3-chloro-1-(3-pyridinyl)-1H-pyrazol-4-yl]-N-ethyl-3-[(3,3,3-trifluoropropyl)thio]-propanamide (known from WO 2015/058021 A1, WO 2015/058028 A1) (CAS 1477919-27-9) and N-[4-(aminothioxomethyl)-2-methyl-6-[(methylamino)carbonyl]phenyl]-3-bromo-1-(3-chloro-2-pyridinyl)-1H-pyrazole-5-carboxamide (known from CN 103265527 A) (CAS 1452877-50-7), 5-(1,3-dioxan-2-yl)-4-[[4-(trifluoromethyl)phenyl]methoxy]-pyrimidine (known from WO 2013/115391 A1) (CAS 1449021-97-9), 3-(4-chloro-2,6-dimethylphenyl)-4-hydroxy-8-methoxy-1-methyl-1,8-diazaspiro[4.5]dec-3-en-2-one (known from WO 2010/066780 A1, WO 2011/151146 A1) (CAS 1229023-34-0), 3-(4-chloro-2,6-dimethylphenyl)-8-methoxy-1-methyl-1,8-diazaspiro[4.5]decane-2,4-dione (known from WO 2014/187846 A1) (CAS 1638765-58-8), 3-(4-chloro-2,6-dimethylphenyl)-8-methoxy-1-methyl-2-oxo-1,8-diazaspiro[4.5]dec-3-en-4-yl-carbonic acid ethyl ester (known from WO 2010/066780 A1, WO 2011151146 A1) (CAS 1229023-00-0), N-[1-[(6-chloro-3-pyridinyl)methyl]-2(1H)-pyridinylidene]-2,2,2-trifluoro-acetamide (known from DE 3639877 A1, WO 2012029672 A1) (CAS 1363400-41-2), [N(E)]-N-[1-[(6-chloro-3-pyridinyl)methyl]-2(1H)-pyridinylidene]-2,2,2-trifluoro-acetamide, (known from WO 2016005276 A1) (CAS 1689566-03-7), [N(Z)]-N-[1-[(6-chloro-3-pyridinyl)methyl]-2(1H)-pyridinylidene]-2,2,2-trifluoro-acetamide, (CAS 1702305-40-5), 3-endo-3-[2-propoxy-4-(trifluoromethyl)phenoxy]-9-[[5-(trifluoromethyl)-2-pyridinyl]oxy]-9-azabicyclo[3.3.1]nonane (known from WO 2011/105506 A1, WO 2016/133011 A1) (CAS 1332838-17-1). Examples of safeners which could be mixed with the compound of formula (I) and composition comprising thereof are, for example, benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}-sulfonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

Examples of herbicides which could be mixed with the compound of formula (I) and composition comprising thereof are:

Acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate, and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamin, -ethyl, -2-ethylhexyl, -isobutyl, -isooctyl, -isopropylammonium, -potassium, -triisopropanolammonium, and -trolamine, 2,4-DB, 2,4-DB-butyl, -dimethylammonium, -isooctyl, -potassium, and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, etha-metsulfuron-methyl, ethiozin, ethofumesate, ethoxyfen, ethoxyfen-ethyl, ethoxysulfuron, etobenzanid, F-9600, F-5231, i.e. N-{2-chloro-4-fluoro-5-[4-(3-fluoropropyl)-5-oxo-4,5-dihydro-1H-tetrazol-1-yl]phenyl}ethanesulfonamide, F-7967, i. e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium, and -trimesium, H-9201, i.e. O-(2,4-dimethyl-6-nitrophenyl)O-ethyl isopropylphosphoramidothioate, halauxifen, halauxifen-methyl halosafen, halosulfuron, halosulfuron-methyl, haloxyfop, haloxyfop-P, haloxyfop-ethoxyethyl, haloxyfop-P-ethoxyethyl, haloxyfop-methyl, haloxy-fop-P-methyl, hexazinone, HW-02, i.e. 1-(dimethoxyphosphoryl) ethyl-(2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and -sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium, and -sodium, MCPB, MCPB-methyl, -ethy,l and -sodium, mecoprop, mecoprop-sodium, and -butotyl, mecoprop-P, mecoprop-P-butotyl, -dimethylammonium, -2-ethylhexyl, and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, i.e. N-(3-chloro-4-isopropylphenyl)-2-methylpentan amide, NGGC-011, napropamide, NC-310, i.e. [5-(benzyloxy)-1-methyl-1H-pyrazol-4-yl](2,4-dichlorophenyl)methanone, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trichloroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and the following compounds:

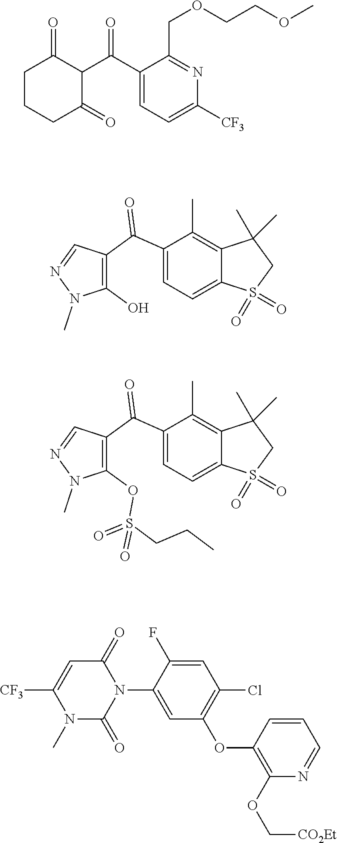 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=O)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl]

Examples for plant growth regulators are:

Acibenzolar, acibenzolar-S-methyl, 5-aminolevulinic acid, ancymidol, 6-benzylaminopurine, Brassinolid, catechine, chlormequat chloride, cloprop, cyclanilide, 3-(cycloprop-1-enyl) propionic acid, daminozide, dazomet, n-decanol, dikegulac, dikegulac-sodium, endothal, endothal-dipotassium, -disodium, and -mono(N,N-dimethylalkylammonium), ethephon, flumetralin, flurenol, flurenol-butyl, flurprimidol, forchlorfenuron, gibberellic acid, inabenfide, indol-3-acetic acid (IAA), 4-indol-3-ylbutyric acid, isoprothiolane, probenazole, jasmonic acid, maleic hydrazide, mepiquat chloride, 1-methylcyclopropene, methyl jasmonate, 2-(1-naphthyl)acetamide, 1-naphthylacetic acid, 2-naphthyloxyacetic acid, nitrophenolate-mixture, paclobutrazol, N-(2-phenylethyl)-beta-alanine, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

### METHODS AND USES

The compound of formula (I) and composition comprising thereof have potent microbicidal activity and/or plant defense modulating potential. They can be used for controlling unwanted microorganisms, such as unwanted fungi, oomycetes and bacteria. They can be particularly useful in crop protection (they control microorganisms that cause plants diseases) or for protecting materials (e.g. industrial materials, timber, storage goods) as described in more details herein below. More specifically, the compound of formula (I) and composition comprising thereof can be used to protect seeds, germinating seeds, emerged seedlings, plants, plant parts, fruits, harvest goods and/or the soil in which the plants grow from unwanted microorganisms.

Control or controlling as used herein encompasses protective, curative and eradicative treatment of unwanted microorganisms. Unwanted microorganisms may be pathogenic bacteria, pathogenic virus, pathogenic oomycetes or pathogenic fungi, more specifically phytopathogenic bacteria, phytopathogenic virus, phytopathogenic oomycetes or phytopathogenic fungi. As detailed herein below, these phytopathogenic microorganims are the causal agents of a broad spectrum of plants diseases.

More specifically, the compound of formula (I) and composition comprising thereof can be used as fungicides. For the purpose of the specification, the term “fungicide” refers to a compound or composition that can be used in crop protection for the control of unwanted fungi, such as Plasmodiophoromycetes, Chytridiomycetes, Zygomycetes, Ascomycetes, Basidiomycetes and Deuteromycetes and/or for the control of Oomycetes.

The compound of formula (I) and composition comprising thereof may also be used as antibacterial agent. In particular, they may be used in crop protection, for example for the control of unwanted bacteria, such as Pseudomonadaceae, Rhizobiaceae, Xanthomonadaceae, Enterobacteriaceae, Corynebacteriaceae and Streptomycetaceae.

The compound of formula (I) and composition comprising thereof may also be used as antiviral agent in crop protection. For example the compound of formula (I) and composition comprising thereof may have effects on diseases from plant viruses, such as the tobacco mosaic virus (TMV), tobacco rattle virus, tobacco stunt virus (TStuV), tobacco leaf curl virus (VLCV), tobacco nervilia mosaic virus (TVBMV), tobacco necrotic dwarf virus (TNDV), tobacco streak virus (TSV), potato virus X (PVX), potato viruses Y, S, M, and A, potato acuba mosaic virus (PAMV), potato mop-top virus (PMTV), potato leaf-roll virus (PLRV), alfalfa mosaic virus (AMV), cucumber mosaic virus (CMV), cucumber green mottlemosaic virus (CGMMV), cucumber yellows virus (CuYV), watermelon mosaic virus (WMV), tomato spotted wilt virus (TSWV), tomato ringspot virus (TomRSV), sugarcane mosaic virus (SCMV), rice drawf virus, rice stripe virus, rice black-streaked drawf virus, strawberry mottle virus (SMoV), strawberry vein banding virus (SVBV), strawberry mild yellow edge virus (SMYEV), strawberry crinkle virus (SCrV), broad beanwilt virus (BBWV), and melon necrotic spot virus (MNSV).

The present invention also relates to a method for controlling unwanted phytopathogenic microorganisms, such as unwanted fungi, oomycetes and bacteria, comprising the step of applying at least one compound of formula (I) or at least one composition to the plants, plant parts, seeds, fruits or to the soil in which the plants grow.

Typically, when the compound of formula (I) and composition comprising thereof are used in curative or protective methods for controlling phytopathogenic fungi and/or phytopathogenic oomycetes, an effective and plant-compatible amount thereof is applied to the plants, plant parts, fruits, seeds or to the soil or substrates in which the plants grow. Suitable substrates that may be used for cultivating plants include inorganic based substrates, such as mineral wool, in particular stone wool, perlite, sand or gravel; organic substrates, such as peat, pine bark or sawdust; and petroleum based substrates such as polymeric foams or plastic beads. Effective and plant-compatible amount means an amount that is sufficient to control or destroy the fungi present or liable to appear on the cropland and that does not entail any appreciable symptom of phytotoxicity for said crops. Such an amount can vary within a wide range depending on the fungus to be controlled, the type of crop, the crop growth stage, the climatic conditions and the respective compound of formula (I) or composition used. This amount can be determined by systematic field trials that are within the capabilities of a person skilled in the art.

Plants and Plant Parts

The compound of formula (I) and composition comprising thereof may be applied to any plants or plant parts.

Plants mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the genetically modified plants (GMO or transgenic plants) and the plant cultivars which are protectable and non-protectable by plant breeders' rights.

Genetically Modified Plants (GMO)

Genetically modified plants (GMO or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome. This gene gives the transformed plant new or improved agronomic or other properties by expressing a protein or polypeptide of interest or by downregulating or silencing other gene(s) which are present in the plant (using for example, antisense technology, cosuppression technology, RNA interference-RNAi-technology or microRNA-miRNA-technology). A heterologous gene that is located in the genome is also called a transgene. A transgene that is defined by its particular location in the plant genome is called a transformation or transgenic event.

Plant cultivars are understood to mean plants which have new properties (“traits”) and have been obtained by conventional breeding, by mutagenesis or by recombinant DNA techniques. They can be cultivars, varieties, bio- or genotypes.

Plant parts are understood to mean all parts and organs of plants above and below the ground, such as shoots, leaves, needles, stalks, stems, flowers, fruit bodies, fruits, seeds, roots, tubers and rhizomes. The plant parts also include harvested material and vegetative and generative propagation material, for example cuttings, tubers, rhizomes, slips and seeds.

Plants which may be treated in accordance with the methods described herein include the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); useful plants and ornamental plants for gardens and wooded areas; and genetically modified varieties of each of these plants.

Plants and plant cultivars which may be treated by the above disclosed methods include plants and plant cultivars which are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may be treated by the above disclosed methods include those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may be treated by the above disclosed methods include those plants characterized by enhanced yield characteristics. Increased yield in said plants may be the result of, for example, improved plant physiology, growth and development, such as water use efficiency, water retention efficiency, improved nitrogen use, enhanced carbon assimilation, improved photosynthesis, increased germination efficiency and accelerated maturation. Yield may furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content and composition for example cotton or starch, protein content, oil content and composition, nutritional value, reduction in anti-nutritional compounds, improved processability and better storage stability.

Plants and plant cultivars which may be treated by the above disclosed methods include plants and plant cultivars which are hybrid plants that already express the characteristic of heterosis or hybrid vigor which results in generally higher yield, vigor, health and resistance towards biotic and abiotic stresses.

Plants and plant cultivars (obtained by plant biotechnology methods such as genetic engineering) which may be treated by the above disclosed methods include plants and plant cultivars which are herbicide-tolerant plants, i.e. plants made tolerant to one or more given herbicides. Such plants can be obtained either by genetic transformation, or by selection of plants containing a mutation imparting such herbicide tolerance.

Plants and plant cultivars (obtained by plant biotechnology methods such as genetic engineering) which may be treated by the above disclosed methods include plants and plant cultivars which are insect-resistant transgenic plants, i.e. plants made resistant to attack by certain target insects. Such plants can be obtained by genetic transformation, or by selection of plants containing a mutation imparting such insect resistance.

Plants and plant cultivars (obtained by plant biotechnology methods such as genetic engineering) which may be treated by the above disclosed methods include plants and plant cultivars which are disease-resistant transgenic plants, i.e. plants made resistant to attack by certain target insects. Such plants can be obtained by genetic transformation, or by selection of plants containing a mutation imparting such insect resistance.

Plants and plant cultivars (obtained by plant biotechnology methods such as genetic engineering) which may be treated by the above disclosed methods include plants and plant cultivars which are tolerant to abiotic stresses. Such plants can be obtained by genetic transformation, or by selection of plants containing a mutation imparting such stress resistance.

Plants and plant cultivars (obtained by plant biotechnology methods such as genetic engineering) which may be treated by the above disclosed methods include plants and plant cultivars which show altered quantity, quality and/or storage-stability of the harvested product and/or altered properties of specific ingredients of the harvested product.

Plants and plant cultivars (obtained by plant biotechnology methods such as genetic engineering) which may be treated by the above disclosed methods include plants and plant cultivars, such as cotton plants, with altered fiber characteristics. Such plants can be obtained by genetic transformation, or by selection of plants contain a mutation imparting such altered fiber characteristics.

Plants and plant cultivars (obtained by plant biotechnology methods such as genetic engineering) which may be treated by the above disclosed methods include plants and plant cultivars, such as oilseed rape or related Brassica plants, with altered oil profile characteristics. Such plants can be obtained by genetic transformation, or by selection of plants contain a mutation imparting such altered oil profile characteristics.

Plants and plant cultivars (obtained by plant biotechnology methods such as genetic engineering) which may be treated by the above disclosed methods include plants and plant cultivars, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering.

Plants and plant cultivars (obtained by plant biotechnology methods such as genetic engineering) which may be treated by the above disclosed methods include plants and plant cultivars, such as Tobacco plants, with altered post-translational protein modification patterns.

Pathogens

Non-limiting examples of pathogens of diseases which may be treated in accordance with the invention include:

diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator;
diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus;
diseases caused by pathogens from the group of the Oomycetes, for example Albugo species, for example Albugo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum; leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidial form: Drechslera, syn: Helminthosporium) or Cochliobolus miyabeanus; Colletotrichum species, for example Colletotrichum lindemuthanium; Corynespora species, for example Corynespora cassiicola; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans; Magnaporthe species, for example Magnaporthe grisea; Microdochium species, for example Microdochium nivale; Mycosphaerella species, for example Mycosphaerella graminicola, Mycosphaerella arachidicola or Mycosphaerella fijiensis; Phaeosphaeria species, for example Phaeosphaeria nodorum; Pyrenophora species, for example Pyrenophora teres or Pyrenophora tritici repentis; Ramularia species, for example Ramularia collo-cygni or Ramularia areola; Rhynchosporium species, for example Rhynchosporium secalis; Septoria species, for example Septoria apii or Septoria lycopersici; Stagonospora species, for example Stagonospora nodorum; Typhula species, for example Typhula incarnata; Venturia species, for example Venturia inaequalis; root and stem diseases caused, for example, by Corticium species, for example Corticium graminearum; Fusarium species, for example Fusarium oxysporum; Gaeumannomyces species, for example Gaeumannomyces graminis; Plasmodiophora species, for example Plasmodiophora brassicae; Rhizoctonia species, for example Rhizoctonia solani; Sarocladium species, for example Sarocladium oryzae; Sclerotium species, for example Sclerotium oryzae; Tapesia species, for example Tapesia acuformis; Thielaviopsis species, for example Thielaviopsis basicola;
ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Stagnospora species, for example Stagnospora nodorum;
diseases caused by smut fungi, for example Sphacelotheca species, for example Sphacelotheca reiliana; Tilletia species, for example Tilletia caries or Tilletia controversa; Urocystis species, for example Urocystis occulta; Ustilago species, for example Ustilago nuda;
fruit rot caused, for example, by Aspergillus species, for example Aspergillus flavus; Botrytis species, for example Botrytis cinerea; Monilinia species, for example Monilinia laxa; Penicillium species, for example Penicillium expansum or Penicillium purpurogenum; Rhizopus species, for example Rhizopus stolonifer; Sclerotinia species, for example Sclerotinia sclerotiorum; Verticilium species, for example Verticilium alboatrum;
seed- and soil-borne rot and wilt diseases, and also diseases of seedlings, caused, for example, by Alternaria species, for example Alternaria brassicicola; Aphanomyces species, for example Aphanomyces euteiches; Ascochyta species, for example Ascochyta lentis; Aspergillus species, for example Aspergillus flavus; Cladosporium species, for example Cladosporium herbarum; Cochliobolus species, for example Cochliobolus sativus (conidial form: Drechslera, Bipolaris Syn: Helminthosporium); Colletotrichum species, for example Colletotrichum coccodes; Fusarium species, for example Fusarium culmorum; Gibberella species, for example Gibberella zeae; Macrophomina species, for example Macrophomina phaseolina; Microdochium species, for example Microdochium nivale; Monographella species, for example Monographella nivalis; Penicillium species, for example Penicillium expansum; Phoma species, for example Phoma lingam; Phomopsis species, for example Phomopsis sojae; Phytophthora species, for example Phytophthora cactorum; Pyrenophora species, for example Pyrenophora graminea; Pyricularia species, for example Pyricularia oryzae; Pythium species, for example Pythium ultimum; Rhizoctonia species, for example Rhizoctonia solani; Rhizopus species, for example Rhizopus oryzae; Sclerotium species, for example Sclerotium rolfsii; Septoria species, for example Septoria nodorum; Typhula species, for example Typhula incarnata; Verticillium species, for example Verticillium dahliae;
cancers, galls and witches' broom caused, for example, by Nectria species, for example Nectria galligena; wilt diseases caused, for example, by Verticillium species, for example Verticillium longisporum; Fusarium species, for example Fusarium oxysporum;
deformations of leaves, flowers and fruits caused, for example, by Exobasidium species, for example Exobasidium vexans; Taphrina species, for example Taphrina deformans;
degenerative diseases in woody plants, caused, for example, by Esca species, for example Phaeomoniella chlamydospora, Phaeoacremonium aleophilum or Fomitiporia mediterranea; Ganoderma species, for example Ganoderma boninense;
diseases of plant tubers caused, for example, by Rhizoctonia species, for example Rhizoctonia solani; Helminthosporium species, for example Helminthosporium solani;
diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora; Liberibacter species, for example Liberibacter asiaticus; Xyella species, for example Xylella fastidiosa; Ralstonia species, for example Ralstonia solanacearum; Dickeya species, for example Dickeya solani; Clavibacter species, for example Clavibacter michiganensis; Streptomyces species, for example Streptomyces scabies.
diseases of soya beans:
Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), Choanephora leaf blight (Choanephora infundibulifera trispora (Syn.)), Dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), sudden death syndrome (Fusarium virguliforme), target spot (Corynespora cassiicola).
Fungal diseases on roots and the stem base caused, for example, by black root rot (Calonectria crotalariae), charcoal rot (Macrophomina phaseolina), Fusarium blight or wilt, root rot, and pod and collar rot (Fusarium oxysporum, Fusarium orthoceras, Fusarium semitectum, Fusarium equiseti), Mycoleptodiscus root rot (Mycoleptodiscus terrestris), Neocosmospora (Neocosmospora vasinfecta), pod and stem blight (Diaporthe phaseolorum), stem canker (Diaporthe phaseolorum var. caulivora), Phytophthora rot (Phytophthora megasperma), brown stem rot (Phialophora gregata), Pythium rot (Pythium aphanidermatum, Pythium irregulare, Pythium debaryanum, Pythium myriotylum, Pythium ultimum), Rhizoctonia root rot, stem decay, and damping-off (Rhizoctonia solani), Sclerotinia stem decay (Sclerotinia sclerotiorum), Sclerotinia southern blight (Sclerotinia rolfsii), Thielaviopsis root rot (Thielaviopsis basicola).

Mycotoxins

In addition, the compound of formula (I) and composition comprising thereof may reduce the mycotoxin content in the harvested material and the foods and feeds prepared therefrom. Mycotoxins include particularly, but not exclusively, the following: deoxynivalenol (DON), nivalenol, 15-Ac-DON, 3-Ac-DON, T2- and HT2-toxin, fumonisins, zearalenon, moniliformin, fusarin, diaceotoxyscirpenol (DAS), beauvericin, enniatin, fusaroproliferin, fusarenol, ochratoxins, patulin, ergot alkaloids and aflatoxins which can be produced, for example, by the following fungi: Fusarium spec., such as F. acuminatum, F. asiaticum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides etc., and also by Aspergillus spec., such as A. flavus, A. parasiticus, A. nomius, A. ochraceus, A. clavatus, A. terreus, A. versicolor, Penicillium spec., such as P. verrucosum, P. viridicatum, P. citrinum, P. expansum, P. claviforme, P. roqueforti, Claviceps spec., such as C. purpurea, C. fusiformis, C. paspali, C. africana, Stachybotrys spec. and others.

Material Protection

The compound of formula (I) and composition comprising thereof may also be used in the protection of materials, especially for the protection of industrial materials against attack and destruction by phytopathogenic fungi.

In addition, the compound of formula (I) and composition comprising thereof may be used as antifouling compositions, alone or in combinations with other active ingredients.

Industrial materials in the present context are understood to mean inanimate materials which have been prepared for use in industry. For example, industrial materials which are to be protected from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by microorganisms. Parts of production plants and buildings, for example cooling-water circuits, cooling and heating systems and ventilation and air-conditioning units, which may be impaired by the proliferation of microorganisms may also be mentioned within the scope of the materials to be protected.

Industrial materials within the scope of the present invention preferably include adhesives, sizes, paper and card, leather, wood, paints, cooling lubricants and heat transfer fluids, more preferably wood.

The compound of formula (I) and composition comprising thereof may prevent adverse effects, such as rotting, decay, discoloration, decoloration or formation of mould.

In the case of treatment of wood the compound of formula (I) and composition comprising thereof may also be used against fungal diseases liable to grow on or inside timber.

Timber means all types of species of wood, and all types of working of this wood intended for construction, for example solid wood, high-density wood, laminated wood, and plywood. In addition, the compound of formula (I) and composition comprising thereof may be used to protect objects which come into contact with saltwater or brackish water, especially hulls, screens, nets, buildings, moorings and signalling systems, from fouling.

The compound of formula (I) and composition comprising thereof may also be employed for protecting storage goods. Storage goods are understood to mean natural substances of vegetable or animal origin or processed products thereof which are of natural origin, and for which long-term protection is desired. Storage goods of vegetable origin, for example plants or plant parts, such as stems, leaves, tubers, seeds, fruits, grains, may be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The compound of formula (I) and composition comprising thereof may prevent adverse effects, such as rotting, decay, discoloration, decoloration or formation of mould.

Microorganisms capable of degrading or altering industrial materials include, for example, bacteria, fungi, yeasts, algae and slime organisms. The compound of formula (I) and composition comprising thereof preferably act against fungi, especially moulds, wood-discoloring and wood-destroying fungi (Ascomycetes, Basidiomycetes, Deuteromycetes and Zygomycetes), and against slime organisms and algae. Examples include microorganisms of the following genera: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

Seed Treatment

The compound of formula (I) and composition comprising thereof may also be used to protect seeds from unwanted microorganisms, such as phytopathogenic microorganisms, for instance phytopathogenic fungi or phytopathogenic oomycetes. The term seed(s) as used herein include dormant seeds, primed seeds, pregerminated seeds and seeds with emerged roots and leaves.

Thus, the present invention also relates to a method for protecting seeds from unwanted microorganisms which comprises the step of treating the seeds with the compound of formula (I) or the composition.

The treatment of seeds with the compound of formula (I) or the composition protects the seeds from phytopathogenic microorganisms, but also protects the germinating seeds, the emerging seedlings and the plants after emergence from the treated seeds. Therefore, the present invention also relates to a method for protecting seeds, germinating seeds and emerging seedlings.

The seeds treatment may be performed prior to sowing, at the time of sowing or shortly thereafter.

When the seeds treatment is performed prior to sowing (e.g. so-called on-seed applications), the seeds treatment may be performed as follows: the seeds may be placed into a mixer with a desired amount of the compound of formula (I) or the composition, the seeds and the compound of formula (I) or the composition are mixed until an homogeneous distribution on seeds is achieved. If appropriate, the seeds may then be dried.

The invention also relates to seeds coated with the compound of formula (I) or composition comprising thereof.

Preferably, the seeds are treated in a state in which it is sufficiently stable for no damage to occur in the course of treatment. In general, seeds can be treated at any time between harvest and shortly after sowing. It is customary to use seeds which have been separated from the plant and freed from cobs, shells, stalks, coats, hairs or the flesh of the fruits. For example, it is possible to use seeds which have been harvested, cleaned and dried down to a moisture content of less than 15% by weight. Alternatively, it is also possible to use seeds which, after drying, for example, have been treated with water and then dried again, or seeds just after priming, or seeds stored in primed conditions or pre-germinated seeds, or seeds sown on nursery trays, tapes or paper.

The amount of the compound of formula (I) or composition comprising thereof applied to the seeds is typically such that the germination of the seed is not impaired, or that the resulting plant is not damaged.

This must be ensured particularly in case the the compound of formula (I) would exhibit phytotoxic effects at certain application rates. The intrinsic phenotypes of transgenic plants should also be taken into consideration when determining the amount of the compound of formula (I) to be applied to the seed in order to achieve optimum seed and germinating plant protection with a minimum amount of compound being employed.

The compound of formula (I) can be applied as such, directly to the seeds, i.e. without the use of any other components and without having been diluted. Also the composition comprising thereof can be applied to the seeds.

The compound of formula (I) and composition comprising thereof are suitable for protecting seeds of any plant variety. Preferred seeds are that of cereals (such as wheat, barley, rye, millet, triticale, and oats), oilseed rape, maize, cotton, soybean, rice, potatoes, sunflower, beans, coffee, peas, beet (e.g. sugar beet and fodder beet), peanut, vegetables (such as tomato, cucumber, onions and lettuce), lawns and ornamental plants. More preferred are seeds of wheat, soybean, oilseed rape, maize and rice.

The compound of formula (I) and composition comprising thereof may be used for treating transgenic seeds, in particular seeds of plants capable of expressing a polypeptide or protein which acts against pests, herbicidal damage or abiotic stress, thereby increasing the protective effect. Seeds of plants capable of expressing a polypeptide or protein which acts against pests, herbicidal damage or abiotic stress may contain at least one heterologous gene which allows the expression of said polypeptide or protein. These heterologous genes in transgenic seeds may originate, for example, from microorganisms of the species Bacillus, Rhizobium, Pseudomonas, Serratia, Trichoderma, Clavibacter, Glomus or Gliocladium. These heterologous genes preferably originate from Bacillus sp., in which case the gene product is effective against the European corn borer and/or the Western corn rootworm. Particularly preferably, the heterologous genes originate from Bacillus thuringiensis.

Application

The compound of formula (I) can be applied as such, or for example in the form of as ready-to-use solutions, emulsions, water- or oil-based suspensions, powders, wettable powders, pastes, soluble powders, dusts, soluble granules, granules for broadcasting, suspoemulsion concentrates, natural products impregnated with the compound of formula (I), synthetic substances impregnated with the compound of formula (I), fertilizers or microencapsulations in polymeric substances.

Application is accomplished in a customary manner, for example by watering, spraying, atomizing, broadcasting, dusting, foaming, spreading-on and the like. It is also possible to deploy the compound of formula (I) by the ultra-low volume method, via a drip irrigation system or drench application, to apply it in-furrow or to inject it into the soil stem or trunk. It is further possible to apply the compound of formula (I) by means of a wound seal, paint or other wound dressing.

The effective and plant-compatible amount of the compound of formula (I) which is applied to the plants, plant parts, fruits, seeds or soil will depend on various factors, such as the compound/composition employed, the subject of the treatment (plant, plant part, fruit, seed or soil), the type of treatment (dusting, spraying, seed dressing), the purpose of the treatment (curative and protective), the type of microorganisms, the development stage of the microorganisms, the sensitivity of the microorganisms, the crop growth stage and the environmental conditions.

When the compound of formula (I) is used as a fungicide, the application rates can vary within a relatively wide range, depending on the kind of application. For the treatment of plant parts, such as leaves, the application rate may range from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used). For the treatment of seeds, the application rate may range from 0.1 to 200 g per 100 kg of seeds, preferably from 1 to 150 g per 100 kg of seeds, more preferably from 2.5 to 25 g per 100 kg of seeds, even more preferably from 2.5 to 12.5 g per 100 kg of seeds. For the treatment of soil, the application rate may range from 0.1 to 10 000 g/ha, preferably from 1 to 5000 g/ha.

These application rates are merely examples and are not intended to limit the scope of the present invention.

Aspects of the present teaching may be further understood in light of the following examples, which should not be construed as limiting the scope of the present teaching in any way.

### EXAMPLES

### Preparation Examples

In the following examples, the log P value and mass peak are as defined in table 1.

### Preparation Example 1: 7,8-difluoro-3-[3-fluoro-2-(6-fluoropyridin-3-yl)benzyl]-2-methylquinoline (Compound I.042)

In a 5 mL microwave tube, were weighed 73 mg (0.51 mmol) of (6-fluoropyridin-3-yl)boronic acid, 10 mg (0.047 mmol) of Palladium(II) acetate and 38 mg (0.094 mmol) of dicyclohexyl(2′,6′-dimethoxybiphenyl-2-yl)phosphane. 214 mg (1.41 mmol) of cesium fluoride were then added followed by 172 mg (0.47 mmol) of 3-(2-bromo-3-fluorobenzyl)-7,8-difluoro-2-methylquinoline in solution in 2.9 mL of a 2:1 mixture of 1,2-dimethoxyethane and methanol. The reaction mixture was heated under microwave at 80° C. for 1 hour. The cooled reaction mixture was transferred onto a 2 g silicagel cartridge and eluted three times by 8 mL of dichloromethane. The filtrate was concentrated in vacuo and purified by preparative HPLC (gradient acetonitrile/water+0.1% HCO₂H) to yield 117 mg (61%) of 7,8-difluoro-3-[3-fluoro-2-(6-fluoropyridin-3-yl)benzyl]-2-methylquinoline. Log P=3.70. Mass (M+H)=383.

### Preparation Example 2: N-[2-(4-cyclopropylpyridin-3-yl)-3-fluorophenyl]-7,8-difluoro-2-methylquinolin-3-amine (Compound I.104)

### Step 1: Preparation of N-(2-bromo-3-fluorophenyl)-7,8-difluoro-2-methylquinolin-3-amine

A mixture of 2.5 g (12.23 mmol) of 7,8-difluoro-2-methylquinolin-3-amine and 3.32 g (12.84 mmol) of 1,2-dibromo-3-fluorobenzene together with 11.95 mg (36.69 mmol) of cesium carbonate and 708 mg (1.22 mmol) of 9,9-dimethyl-9H-xanthene-4,5-diyl)bis-(diphenylphosphane [Xantphos] in 70 mL of 1,4-dioxane was degassed with argon for 30 minutes. 896 mg (0.97 mmol) of tris(dibenzylideneacetone)palladium(0) was added and the reaction mixture was heated at reflux for 26 hours. The cooled reaction mixture was filtered over a silica gel pad, and the pad washed by ethyl acetate. The organic phase was concentrated under vacuo and purified by column chromatography on silica gel (300 g cartridge—gradient n-heptane/ethyl acetate) to yield 2.31 g (49%) of N-(2-bromo-3-fluorophenyl)-7,8-difluoro-2-methylquinolin-3-amine. Log P=3.28. Mass (M+H)=367.

### Step 2: Preparation of N-[2-(4-cyclopropylpyridin-3-yl)-3-fluorophenyl]-7,8-difluoro-2-methylquinolin-3-amine (Compound I.104)

In a 25 mL microwave tube, to a solution of 8 mL of 1,4-dioxane and 1.5 mL of water degassed with argon for 15 minutes, were successively added, 130 mg (0.35 mmol) of N-(2-bromo-3-fluorophenyl)-7,8-difluoro-2-methylquinolin-3-amine, 115 mg (0.70 mmol) of (4-cyclopropylpyridin-3-yl)boronic acid, 134 mg (0.88 mmol) of cesium fluoride, 14.5 mg (0.035 mmol) of tris(dibenzylideneacetone)palladium(0) and 14.5 mg (0.035 mmol) of 2-dicyclohexylphosphino-2′,6′-dimethoxybiphenyl [S-Phos]. The reaction mixture was heated under microwave at 150° C. for 1 hour. The cooled reaction mixture was diluted with ethyl acetate and filtered over a pad of Celite™. The organic phase was washed by brine and dried over magnesium sulfate. The organic phase was concentrated under vacuo and purified by column chromatography on silica gel (12 g cartridge—gradient n-heptane/ethyl acetate) to yield 43 mg (29%) of N-[2-(4-cyclopropylpyridin-3-yl)-3-fluorophenyl]-7,8-difluoro-2-methylquinolin-3-amine. Log P=2.12. Mass (M+H)=406.

### Preparation Example 3: Preparation of 5,6-difluoro-N-[2-(3-fluoropyridin-4-yl)phenyl]-3-methylquinoxalin-2-amine (Compound I.014)

### Step 1: Preparation of 2-(3-fluoropyridin-4-yl)aniline

In a 50 mL microwave tube, to a solution of 20 mL of 1,4-dioxane and 3 mL of water degassed with argon for 15 minutes, were successively added, 900 mg (4.73 mmol) of 2-bromo-3-fluoroaniline, 2.64 g (11.84 mmol) of 3-fluoro-4-(4,4,5,5-tetramethyl-1,3,2-dioxaborolan-2-yl)pyridine, 1798 mg (11.84 mmol) of cesium fluoride, 216 mg (0.237 mmol) of tris(dibenzylideneacetone)palladium(0) and 194 mg (0.474 mmol) of S-Phos. The reaction mixture was heated under microwave at 150° C. for 1 hour. The cooled reaction mixture was diluted with ethyl acetate and filtered over a pad of Celite™. The organic phase was washed by brine and dried over magnesium sulfate. The organic phase was concentrated under vacuo and purified by column chromatography on silica gel (40 g cartridge—gradient n-heptane/ethyl acetate) to yield 775 mg (72%) of 2-(3-fluoropyridin-4-yl)aniline. Log P=2.38. Mass (M+H)=207.

### Step 2: Preparation of 5,6-difluoro-N-[2-(3-fluoropyridin-4-yl)phenyl]-3-methylquinoxalin-2-amine (Compound I.014)

To a mixture of 295 mg (1.22 mmol) of 2-bromo-5-fluoro-3-methylquinoxaline and 252 mg (1.22 mmol) of 2-(3-fluoropyridin-4-yl)aniline together with 1.19 g (3.67 mmol) of cesium carbonate and 70 mg (0.122 mmol) of Xantphos in 25 mL of degassed 1,4-dioxane, were added 31 mg (0.61 mmol) of palladium-(Pi-cinnamyl) chloride dimer. The reaction mixture was heated at reflux for 2.5 hours. The cooled reaction mixture was diluted with ethyl acetate and filtered over a pad of Celite™. The organic phase was concentrated under vacuo and purified by column chromatography on silica gel (12 g cartridge—gradient n-heptane/ethyl acetate) to yield 113 mg (25%) of 5,6-difluoro-N-[2-(3-fluoropyridin-4-yl)phenyl]-3-methylquinoxalin-2-amine as a pale yellow solid. Log P=2.83. Mass (M+H)=367.

### Preparation Example 4: Preparation of 7,8-difluoro-3-{[4-(2-fluoropyridin-4-yl)-3-thienyl]methyl}-2-methylquinoline (Compound I.138)

### Step 1: Preparation of 3-bromo-4-(chloromethyl)thiophene

To a mixture of 1 g (5.02 mmol) of (4-bromo-3-thienyl)methanol and 770 mg (7.53 mmol) of triethylamine in 20 mL of dichloromethane were added 867 mg (7.53 mmol) of methanesulfonyl chloride. The reaction mixture was stirred overnight at ambient temperature. The mixture was concentrated in vacuo to give 3.19 g of residue as an orange oil. The residue was purified by column chromatography on silica gel (40 g cartridge-gradient n-heptane/ethyl acetate) to yield 1.01 g (95%) of 3-bromo-4-(chloromethyl)thiophene as a light yellow oil. Log P=2.99. Mass (M+H): no ionization. Mass (M) by GC-mass=210.

### Step 2: Preparation of 3-[(4-bromo-3-thienyl)methyl]-7,8-difluoro-2-methylquinoline

In a 5 mL microwave tube, were dissolved 99 mg (0.43 mmol) of (7,8-difluoro-2-methylquinolin-3-yl)boronic acid and 100 mg (0.47 mmol) of 3-bromo-4-(chloromethyl)thiophene in 4 mL of 1,4-dioxane. A solution of 178 mg (1.29 mmol) of potassium carbonate in 1 mL of water was added and the reaction mixture was degassed 5 minutes with argon. 24.8 mg (0.021 mmol) of tetrakis(triphenyl-phosphine)palladium(0) were further added and the reaction mixture was heated under microwave at 100° C. for 20 minutes. The same reaction was repeated 4 more times. The combined 5 reaction mixtures were poured over 100 mL of water and reextracted by ethyl acetate. The organic phase was dried over magnesium sulfate and concentrated in vacuo to give 978 mg of residue as an orange solid. The residue was purified by column chromatography on silica gel (80 g cartridge—gradient n-heptane/ethyl acetate) to yield 609 mg (80%) of 3-[(4-bromo-3-thienyl)methyl]-7,8-difluoro-2-methylquinoline as a pale yellow solid. Log P=4.01. Mass (M+H)=354.

### Step 3: Preparation of 7,8-difluoro-3-{[4-(2-fluoropyridin-4-yl)-3-thienyl]methyl}-2-methylquinoline (Compound I.138)

In a 5 mL microwave tube, were dissolved 100 mg (0.28 mmol) of 3-[(4-bromo-3-thienyl)methyl]-7,8-difluoro-2-methylquinoline and 265 mg (1.12 mmol) of 2-fluoro-4-(4,4,5,5-tetramethyl-1,3,2-dioxaborolan-2-yl)pyridine in 5 mL of 1,4-dioxane. 129 mg (0.84 mmol) of cesium fluoride together with 12 mg (0.028 mmol) of S-Phos were added and the reaction mixture was degassed 5 minutes with argon. 13 mg (0.014 mmol) of tris(dibenzylideneacetone)palladium(0 were further added and the reaction mixture was heated under microwave at 150° C. for 3 hours. The cooled reaction mixture was filtered over a silica gel pad, and the pad washed by ethyl acetate. The organic phase was concentrated under vacuo and purified by column chromatography on silica gel (40 g cartridge—gradient n-heptane/ethyl acetate) to yield 87 mg (78%) of 7,8-difluoro-3-{[4-(2-fluoropyridin-4-yl)-3-thienyl]methyl}-2-methylquinoline as an orange solid. Log P=3.64. Mass (M+H)=371.

Table 1 illustrates in a non-limiting manner examples of compounds of formula (I):

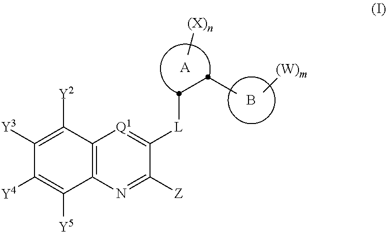

wherein A can be selected in the list consisting of the following groups: A-G1, A-G2, A-G3, A-G4, A-G5, A-G6 and A-G7:

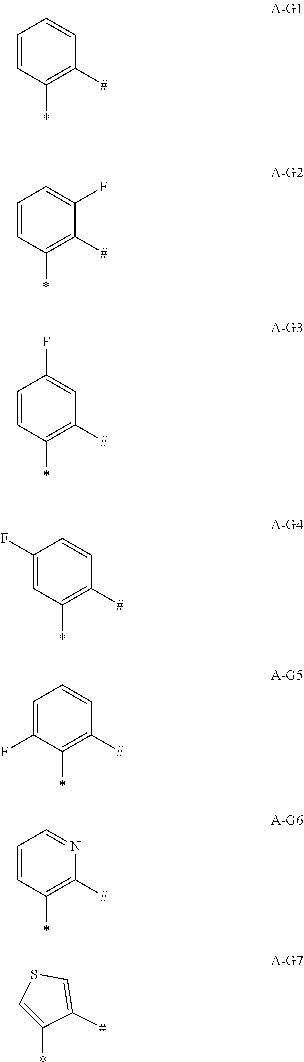

wherein the group A-Gn is connected to L of formula (I) via the bonds identified with “*” and the group A-Gn is connected to the B ring of formula (I) via the bonds identified with “#”.

The compounds as shown in table 1 were prepared in analogy with the examples provided above.

In table 1, the log P values were determined in accordance with EEC Directive 79/831 Annex V.A8 by HPLC (High Performance Liquid Chromatography) on a reversed-phase column (C 18), using the method described below:

temperature: 40° C.; mobile phases: 0.1% aqueous formic acid and acetonitrile; linear gradient from 10% acetonitrile to 95% acetonitrile;

If more than one Log P value is available within the same method, all the values are given and separated by “;”.

Calibration was carried out using unbranched alkan-2-ones (comprising 3 to 16 carbon atoms) with known log P values (determination of the log P values by the retention times using linear interpolation between two successive alkanones). lambda-max-values were determined using UV-spectra from 200 nm to 400 nm and the peak values of the chromatographic signals.

TABLE 1

| Example | 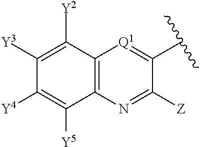 | L | 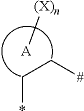 | 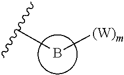 | M + H | logP |
|---|---|---|---|---|---|---|
| I.001 | quinolin-3-yl | NH | A-G3 | pyridin-3-yl | 316 | 1.39 |
| I.002 | 3-methylquinoxalin-2-yl | NH | A-G2 | 3-fluoropyridin-4-yl | 349 | 2.57 |
| I.003 | 7,8-difluoro-2-methylquinolin-3-yl | NH | A-G1 | pyridazin-4-yl | 349 | 2.28 |
| I.004 | 7,8-difluoro-2-methylquinolin-3-yl | NH | A-G1 | 5-methylpyridin-3-yl | 362 | 1.99 |
| I.005 | 7,8-difluoro-2-methylquinolin-3-yl | NH | A-G1 | 2-methylpyridin-4-yl | 362 | 1.75 |
| I.006 | 7,8-difluoro-2-methylquinolin-3-yl | NH | A-G1 | 4-methylpyridin-3-yl | 362 | 1.86 |
| I.007 | 7,8-difluoro-2-methylquinolin-3-yl | CH₂ | A-G2 | pyridin-4-yl | 365 | 2.00 |
| I.008 | 7,8-difluoro-2-methylquinolin-3-yl | CH₂ | A-G2 | pyridin-3-yl | 365 | 2.64 |
| I.009 | 7,8-difluoro-2-methylquinolin-3-yl | NH | A-G2 | pyridin-3-yl | 366 | 2.28 |
| I.010 | 7,8-difluoro-2-methylquinolin-3-yl | NH | A-G2 | pyridin-4-yl | 366 | 2.01 |
| I.011 | 7,8-difluoro-2-methylquinolin-3-yl | CH₂ | A-G2 | pyrimidin-5-yl | 366 | 2.76 |
| I.012 | 8-fluoro-2-methylquinolin-3-yl | NH | A-G4 | 3-fluoropyridin-4-yl | 366 | 2.95 |
| I.013 | 7,8-difluoro-2-methylquinolin-3-yl | NH | A-G2 | pyrimidin-5-yl | 367 | 2.61 |
| I.014 | 5,6-difluoro-3-methylquinoxalin-2-yl | NH | A-G1 | 3-fluoropyridin-4-yl | 367 | 2.83 |
| I.015 | 5-fluoro-3-methylquinoxalin-2-yl | NH | A-G2 | 3-fluoropyridin-4-yl | 367 | 2.67 |
| I.016 | 7,8-difluoro-2-methylquinolin-3-yl | NH | A-G2 | pyridazin-4-yl | 367 | 2.44 |
| I.017 | 7,8-difluoro-2-methylquinolin-3-yl | CH₂ | A-G2 | 3,4-dihydro-2H-pyran-6-yl | 370 | 4.41 |
| I.018 | 7,8-difluoro-2-methylquinolin-3-yl | NH | A-G2 | 3,4-dihydro-2H-pyran-6-yl | 371 | 4.44 |
| I.019 | 7,8-difluoro-2-methylquinolin-3-yl | NH | A-G1 | 6-cyanopyridin-3-yl | 373 | 3.25 |
| I.020 | 7,8-difluoro-2-methylquinolin-3-yl | NH | A-G1 | 5-cyanopyridin-3-yl | 373 | 3.04 |
| I.021 | 7,8-difluoro-2-methylquinolin-3-yl | NH | A-G1 | 3-cyanopyridin-4-yl | 373 | 1.41 |
| I.022 | 7,8-difluoro-2-methylquinolin-3-yl | NH | A-G1 | 3-ethylpyridin-4-yl | 376 | 2.08 |
| I.023 | 7,8-difluoro-2-methylquinolin-3-yl | NH | A-G1 | 4-ethylpyridin-3-yl | 376 | 2.08 |
| I.024 | 7,8-difluoro-2-methylquinolin-3-yl | NH | A-G1 | 2-ethylpyridin-3-yl | 376 | 2.03 |
| I.025 | 7,8-difluoro-2-methylquinolin-3-yl | NH | A-G1 | 3,5-dimethylpyridin-4-yl | 376 | 2.05 |
| I.026 | 7,8-difluoro-2-methylquinolin-3-yl | NH | A-G1 | 1-methyl-6-oxo-1,6-dihydropyridin-2-yl | 378 | 2.52 |
| I.027 | 7,8-difluoro-2-methylquinolin-3-yl | NH | A-G1 | 3-methoxypyridin-4-yl | 378 | 2.14 |
| I.028 | 7,8-difluoro-2-methylquinolin-3-yl | NH | A-G1 | 2-methoxypyridin-4-yl | 378 | 3.56 |
| I.029 | 7,8-difluoro-2-methylquinolin-3-yl | NH | A-G1 | 6-methoxypyridin-2-yl | 378 | 4.66 |
| I.030 | 7,8-difluoro-2-methylquinolin-3-yl | NH | A-G1 | 4-methoxypyridin-3-yl | 378 | 1.77 |
| I.031 | 7,8-difluoro-2-methylquinolin-3-yl | NH | A-G1 | 6-fluoro-5-methylpyridin-3-yl | 380 | 3.69 |
| I.032 | 7,8-difluoro-2-methylquinolin-3-yl | NH | A-G2 | 5-methylpyridin-3-yl | 380 | 2.26 |
| I.033 | 7,8-difluoro-2-methylquinolin-3-yl | NH | A-G2 | 2-methylpyridin-4-yl | 380 | 1.88 |
| I.034 | 7,8-difluoro-2-methylquinolin-3-yl | NH | A-G1 | 3-chloropyridin-4-yl | 382 | 3.37 |
| I.035 | 7,8-difluoro-2-methylquinolin-3-yl | NH | A-G1 | 6-chloropyridin-2-yl | 382 | 4.97 |
| I.036 | 7,8-difluoro-2-methylquinolin-3-yl | NH | A-G1 | 2-chloropyridin-4-yl | 382 | 3.56 |
| I.037 | 7,8-difluoro-2-methylquinolin-3-yl | NH | A-G1 | 6-chloropyridin-3-yl | 382 | 3.62 |
| I.038 | 7,8-difluoro-2-methylquinolin-3-yl | NH | A-G1 | 2-chloropyridin-3-yl | 382 | 3.43 |
| I.039 | 7,8-difluoro-2-methylquinolin-3-yl | NH | A-G1 | 4-chloropyridin-3-yl | 382 | 3.22 |
| I.040 | 7,8-difluoro-2-methylquinolin-3-yl | NH | A-G1 | 5-chloropyridin-3-yl | 382 | 3.54 |
| I.041 | 7,8-difluoro-2-methylquinolin-3-yl | CH₂ | A-G2 | 3-fluoropyridin-4-yl | 383 | 3.40 |
| I.042 | 7,8-difluoro-2-methylquinolin-3-yl | CH₂ | A-G2 | 6-fluoropyridin-3-yl | 383 | 3.70 |
| I.043 | 7,8-difluoro-2-methylquinolin-3-yl | CH₂ | A-G2 | 2-fluoropyridin-3-yl | 383 | 3.55 |
| I.044 | 7,8-difluoro-2-methylquinolin-3-yl | CH₂ | A-G2 | 2-fluoropyridin-4-yl | 383 | 3.65 |
| I.045 | 7,8-difluoro-2-methylquinolin-3-yl | NH | A-G2 | 6-fluoropyridin-3-yl | 384 | 3.46 |
| I.046 | 7,8-difluoro-2-methylquinolin-3-yl | NH | A-G2 | 2-fluoropyridin-4-yl | 384 | 3.46 |
| I.047 | 7,8-difluoro-2-methylquinolin-3-yl | NH | A-G2 | 2-fluoropyridin-3-yl | 384 | 3.35 |
| I.048 | 7,8-difluoro-2-methylquinolin-3-yl | NH | A-G5 | 3-fluoropyridin-4-yl | 384 | 3.04 |
| I.049 | 7,8-difluoro-2-methylquinolin-3-yl | NH | A-G3 | 3-fluoropyridin-4-yl | | 3.24 |
| I.050 | 5,6-difluoro-3-methylquinoxalin-2-yl | CH₂ | A-G2 | 3-fluoropyridin-4-yl | 384 | 3.31 |
| I.051 | 7,8-difluoro-2-methylquinolin-3-yl | NH | A-G2 | 3-fluoropyridin-4-yl | | 3.29 |
| I.052 | 7,8-difluoro-2-methylquinolin-3-yl | NH | A-G1 | 2,5-difluoropyridin-4-yl/ | 384 | 3.62 |
| | | | | 2,5-difluoropyridin-3-yl⁽¹⁾ | | |
| I.053 | 7,8-difluoro-2-methylquinolin-3-yl | NH | A-G1 | 2,3-difluoropyridin-4-yl | 384 | 3.69 |
| I.054 | 7,8-difluoro-2-methylquinolin-3-yl | NH | A-G1 | 2,6-difluoropyridin-4-yl | 384 | 3.91 |
| I.055 | 7,8-difluoro-2-methylquinolin-3-yl | NH | A-G1 | 2,6-difluoropyridin-3-yl | 384 | 3.81 |
| I.056 | 5,6-difluoro-3-methylquinoxalin-2-yl | NH | A-G2 | 3-fluoropyridin-4-yl | 385 | 2.98 |
| I.057 | 7,8-difluoro-2-methylquinolin-3-yl | NH | A-G1 | 4-cyclopropylpyridin-3-yl | 388 | 2.05 |
| I.058 | 7,8-difluoro-2-methylquinolin-3-yl | NH | A-G1 | 3-cyclopropylpyridin-4-yl | 388 | 2.12 |
| I.059 | 7,8-difluoro-2-methylquinolin-3-yl | NH | A-G1 | 2-cyclopropylpyridin-3-yl | 388 | 2.80 |
| I.060 | 7,8-difluoro-2-methylquinolin-3-yl | NH | A-G1 | 2-propylpyridin-3-yl | 390 | 2.21 |
| I.061 | 7,8-difluoro-2-methylquinolin-3-yl | NH | A-G1 | 3-propylpyridin-4-yl | 390 | 2.31 |
| I.062 | 7,8-difluoro-2-methylquinolin-3-yl | NH | A-G1 | 4-propylpyridin-3-yl | 390 | 2.41 |
| I.063 | 7,8-difluoro-2-methylquinolin-3-yl | NH | A-G2 | 3-cyanopyridin-4-yl | 391 | 1.46 |
| I.064 | 7,8-difluoro-2-methylquinolin-3-yl | NH | A-G2 | 5-cyanopyridin-3-yl | 391 | 3.16 |
| I.065 | 7,8-difluoro-2-methylquinolin-3-yl | NH | A-G1 | 6-ethoxypyridin-3-yl | 392 | 4.02 |
| I.066 | 7,8-difluoro-2-methylquinolin-3-yl | NH | A-G1 | 2-ethoxypyridin-3-yl | 392 | 4.24 |
| I.067 | 7,8-difluoro-2-methylquinolin-3-yl | NH | A-G2 | 3-ethylpyridin-4-yl | 394 | 2.41 |
| I.068 | 7,8-difluoro-2-methylquinolin-3-yl | NH | A-G2 | 4-ethylpyridin-3-yl | 394 | 2.41 |
| I.069 | 7,8-difluoro-2-methylquinolin-3-yl | NH | A-G2 | 2-ethylpyridin-3-yl | 394 | 2.41 |
| I.070 | 7,8-difluoro-2-methylquinolin-3-yl | NH | A-G2 | 3,5-dimethylpyridin-4-yl | 394 | 2.28 |
| I.071 | 7,8-difluoro-2-methylquinolin-3-yl | NH | A-G1 | 6-(methylsulfanyl)pyridin-3-yl | 394 | 3.81 |
| I.072 | 7,8-difluoro-2-methylquinolin-3-yl | NH | A-G1 | 2-(methylsulfanyl)pyridin-3-yl | 394 | 4.08 |
| I.073 | 7,8-difluoro-2-methylquinolin-3-yl | CH₂ | A-G2 | 6-methoxypyridin-3-yl | 395 | 3.99 |
| I.074 | 7,8-difluoro-2-methylquinolin-3-yl | CH₂ | A-G2 | 6-methoxypyridin-2-yl | 395 | 3.79 |
| I.075 | 7,8-difluoro-2-methylquinolin-3-yl | CH₂ | A-G2 | 2-methoxypyridin-3-yl | 395 | 3.79 |
| I.076 | 7,8-difluoro-2-methylquinolin-3-yl | NH | A-G2 | 2-methoxypyridin-3-yl | 396 | 3.70 |
| I.077 | 7,8-difluoro-2-methylquinolin-3-yl | NH | A-G2 | 6-methoxypyridin-3-yl | 396 | 3.71 |
| I.078 | 7,8-difluoro-2-methylquinolin-3-yl | CH₂ | A-G2 | 2-methoxypyrimidin-5-yl | 396 | 3.25 |
| I.079 | 7,8-difluoro-2-methylquinolin-3-yl | NH | A-G1 | 2-fluoro-3-methoxypyridin-4-yl | 396 | 3.72 |
| I.080 | 7,8-difluoro-2-methylquinolin-3-yl | NH | A-G2 | 6-methoxypyridin-2-yl | 396 | 4.55 |
| I.081 | 7,8-difluoro-2-methylquinolin-3-yl | NH | A-G2 | 3-methoxypyridin-4-yl | 396 | 2.44 |
| I.082 | 7,8-difluoro-2-methylquinolin-3-yl | NH | A-G2 | 4-methoxypyridin-3-yl | 396 | 1.84 |
| I.083 | 7,8-difluoro-2-methylquinolin-3-yl | NH | A-G2 | 2-methoxypyridin-4-yl | 396 | 3.71 |
| I.084 | 7,8-difluoro-2-methylquinolin-3-yl | NH | A-G1 | 2-chloro-5-methylpyridin-3-yl | 396 | 3.69 |
| I.085 | 7,8-difluoro-2-methylquinolin-3-yl | NH | A-G1 | 6-chloro-2-methylpyridin-3-yl | 396 | 3.94 |
| I.086 | 7,8-difluoro-2-methylquinolin-3-yl | NH | A-G1 | 6-chloro-4-methylpyridin-3-yl | 396 | 3.98 |
| I.087 | 7,8-difluoro-2-methylquinolin-3-yl | NH | A-G1 | 6-chloro-5-methylpyridin-3-yl | 396 | 3.91 |
| I.088 | 7,8-difluoro-2-methylquinolin-3-yl | NH | A-G2 | 2-methoxypyrimidin-5-yl | 397 | 3.08 |
| I.089 | 7,8-difluoro-2-methylquinolin-3-yl | NH | A-G2 | 6-fluoro-5-methylpyridin-3-yl | 398 | 3.75 |
| I.090 | 7,8-difluoro-2-methylquinolin-3-yl | NH | A-G1 | 4-chloro-5-fluoropyridin-3-yl | 400 | 3.62 |
| I.091 | 7,8-difluoro-2-methylquinolin-3-yl | NH | A-G2 | 2-chloropyridin-4-yl | 400 | 3.69 |
| I.092 | 7,8-difluoro-2-methylquinolin-3-yl | NH | A-G2 | 6-chloropyridin-3-yl | 400 | 3.72 |
| I.093 | 7,8-difluoro-2-methylquinolin-3-yl | NH | A-G2 | 3-chloropyridin-4-yl | 400 | 3.46 |
| I.094 | 7,8-difluoro-2-methylquinolin-3-yl | NH | A-G2 | 2-chloropyridin-3-yl | 400 | 3.50 |
| I.095 | 7,8-difluoro-2-methylquinolin-3-yl | NH | A-G1 | 5-chloro-2-fluoropyridin-3-yl | 400 | 4.01 |
| I.096 | 7,8-difluoro-2-methylquinolin-3-yl | NH | A-G1 | 3-chloro-2-fluoropyridin-4-yl | 400 | 3.91 |
| I.097 | 7,8-difluoro-2-methylquinolin-3-yl | NH | A-G1 | 2-chloro-5-fluoropyridin-3-yl | 400 | 3.81 |
| I.098 | 7,8-difluoro-2-methylquinolin-3-yl | NH | A-G1 | 2-chloro-5-fluoropyridin-4-yl | 400 | 3.91 |
| I.099 | 7,8-difluoro-2-methylquinolin-3-yl | NH | A-G2 | 5-chloropyridin-3-yl | 400 | 3.60 |
| I.100 | 7,8-difluoro-2-methylquinolin-3-yl | NH | A-G2 | 2,6-difluoropyridin-4-yl | 402 | 3.98 |
| I.101 | 7,8-difluoro-2-methylquinolin-3-yl | NH | A-G2 | 2,3-difluoropyridin-4-yl | 402 | 3.81 |
| I.102 | 7,8-difluoro-2-methylquinolin-3-yl | NH | A-G2 | 2,5-difluoropyridin-4-yl/ | 402 | 3.77 |
| | | | | 2,5-difluoropyridin-3-yl⁽¹⁾ | | |
| I.103 | 7,8-difluoro-2-methylquinolin-3-yl | NH | A-G2 | 2,6-difluoropyridin-3-yl | 402 | 3.84 |
| I.104 | 7,8-difluoro-2-methylquinolin-3-yl | NH | A-G2 | 4-cyclopropylpyridin-3-yl | 406 | 2.12 |
| I.105 | 7,8-difluoro-2-methylquinolin-3-yl | NH | A-G2 | 2-cyclopropylpyridin-3-yl | 406 | 3.25 |
| I.106 | 7,8-difluoro-2-methylquinolin-3-yl | NH | A-G2 | 3-cyclopropylpyridin-4-yl | 406 | 2.49 |
| I.107 | 7,8-difluoro-2-methylquinolin-3-yl | NH | A-G1 | 6-isopropoxypyridin-3-yl | 406 | 4.43 |
| I.108 | 7,8-difluoro-2-methylquinolin-3-yl | NH | A-G1 | 2,6-dimethoxypyridin-3-yl | 408 | 4.62 |
| I.109 | 7,8-difluoro-2-methylquinolin-3-yl | NH | A-G2 | 2-propylpyridin-3-yl | 408 | 2.62 |
| I.110 | 7,8-difluoro-2-methylquinolin-3-yl | NH | A-G2 | 3-propylpyridin-4-yl | 408 | 2.89 |
| I.111 | 7,8-difluoro-2-methylquinolin-3-yl | NH | A-G2 | 4-propylpyridin-3-yl | 408 | 2.75 |
| I.112 | 7,8-difluoro-2-methylquinolin-3-yl | NH | A-G2 | 6-ethoxypyridin-3-yl | 410 | 4.09 |
| I.113 | 7,8-difluoro-2-methylquinolin-3-yl | NH | A-G2 | 2-ethoxypyridin-3-yl | 410 | 4.13 |
| I.114 | 7,8-difluoro-2-methylquinolin-3-yl | NH | A-G2 | 6-(methylsulfanyl)pyridin-3-yl | 412 | 3.91 |
| I.115 | 7,8-difluoro-2-methylquinolin-3-yl | NH | A-G2 | 2-(methylsulfanyl)pyridin-3-yl | 412 | 3.98 |
| I.116 | 7,8-difluoro-2-methylquinolin-3-yl | NH | A-G1 | 3-chloro-2-methoxypyridin-4-yl | 412 | 4.09 |
| I.117 | 7,8-difluoro-2-methylquinolin-3-yl | NH | A-G2 | 2-fluoro-3-methoxypyridin-4-yl | 414 | 3.75 |
| I.118 | 7,8-difluoro-2-methylquinolin-3-yl | NH | A-G2 | 2-chloro-5-methylpyridin-3-yl | 414 | 3.75 |
| I.119 | 7,8-difluoro-2-methylquinolin-3-yl | NH | A-G2 | 6-chloro-5-methylpyridin-3-yl | 414 | 3.98 |
| I.120 | 7,8-difluoro-2-methylquinolin-3-yl | NH | A-G2 | 6-chloro-2-methylpyridin-3-yl | 414 | 4.01 |
| I.121 | 7,8-difluoro-2-methylquinolin-3-yl | NH | A-G2 | 6-chloro-4-methylpyridin-3-yl | 414 | 4.01 |
| I.122 | 7,8-difluoro-2-methylquinolin-3-yl | NH | A-G1 | 5-(trifluoromethyppyridin-3-yl | 416 | 3.65 |
| I.123 | 7,8-difluoro-2-methylquinolin-3-yl | NH | A-G1 | 6-(trifluoromethyppyridin-3-yl | 416 | 3.91 |
| I.124 | 7,8-difluoro-2-methylquinolin-3-yl | NH | A-G1 | 2,5-dichloropyridin-3-yl | 416 | 4.13 |
| I.125 | 7,8-difluoro-2-methylquinolin-3-yl | NH | A-G1 | 4,5-dichloropyridin-3-yl | 416 | 4.01 |
| I.126 | 7,8-difluoro-2-methylquinolin-3-yl | NH | A-G2 | 2-chloro-5-fluoropyridin-4-yl | 418 | 3.98 |
| I.127 | 7,8-difluoro-2-methylquinolin-3-yl | NH | A-G2 | 2-chloro-5-fluoropyridin-3-yl | 418 | 3.87 |
| I.128 | 7,8-difluoro-2-methylquinolin-3-yl | NH | A-G2 | 5-chloro-2-fluoropyridin-3-yl | 418 | 4.05 |
| I.129 | 7,8-difluoro-2-methylquinolin-3-yl | NH | A-G2 | 3-chloro-2-fluoropyridin-4-yl | 418 | 3.98 |
| I.130 | 7,8-difluoro-2-methylquinolin-3-yl | NH | A-G2 | 6-isopropoxypyridin-3-yl | 424 | 4.47 |
| I.131 | 7,8-difluoro-2-methylquinolin-3-yl | NH | A-G2 | 2,6-dimethoxypyridin-3-yl | 426 | 4.43 |
| I.132 | 7,8-difluoro-2-methylquinolin-3-yl | NH | A-G2 | 3-chloro-2-methoxypyridin-4-yl | 430 | 4.09 |
| I.133 | 7,8-difluoro-2-methylquinolin-3-yl | NH | A-G2 | 5-(trifluoromethyppyridin-3-yl | 434 | 3.75 |
| I.134 | 7,8-difluoro-2-methylquinolin-3-yl | NH | A-G2 | 6-(trifluoromethyppyridin-3-yl | 434 | 3.94 |
| I.135 | 7,8-difluoro-2-methylquinolin-3-yl | NH | A-G1 | 5-bromo-2-methoxypyridin-4-yl | 456 | 4.36 |
| I.136 | 7,8-difluoro-2-methylquinolin-3-yl | NH | A-G2 | 2,5-difluoropyridin-3-yl | | 3.71 |
| I.137 | 7,8-difluoro-2-methylquinolin-3-yl | CH₂ | A-G6 | 2-fluoropyridin-4-yl | 366 | 2.52 |
| I.138 | 7,8-difluoro-2-methylquinolin-3-yl | CH₂ | A-G7 | 2-fluoropyridin-4-yl | 371 | 3.64 |
| I.139 | 8-fluoroquinolin-3-yl | CH₂ | A-G7 | 2-fluoropyridin-4-yl | 339 | 3.11 |

Note (1) : as an inseparable 1:1 mixture

### NMR-Peak Lists

The ¹H-NMR data of selected examples are stated in the form of ¹H-NMR peak lists. For each signal peak, the δ-value in ppm and the signal intensity in brackets are listed.

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

The ¹H-NMR peak lists are similar to classical ¹H-NMR prints and contain therefore usually all peaks, which are listed at classical NMR-interpretation. Additionally they can show like classical ¹H-NMR prints signals of solvents, stereoisomers of the target compounds, which are also object of the invention, and/or peaks of impurities. To show compound signals in the delta-range of solvents and/or water the usual peaks of solvents, for example peaks of DMSO in d6-DMSO and the peak of water are shown in our ¹H-NMR peak lists and have usually on average a high intensity.

The peaks of stereoisomers of the target compounds and/or peaks of impurities have usually on average a lower intensity than the peaks of target compounds (for example with a purity >90%). Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values), can isolate the peaks of the target compounds as needed optionally using additional intensity filters. This isolation would be similar to relevant peak picking at classical ¹H-NMR interpretation.

Further details of NMR-data description with peak lists can be found in the publication “Citation of NMR Peaklist Data within Patent Applications” of the Research Disclosure Database Number 564025.

NMR-Peak Lists for Selected Compounds of Formula (I)

I.001: 1H-NMR(300.2 MHz, CDCl3):
δ = 8.7057 (7.6); 8.7039 (7.6); 8.6985 (8.1); 8.6081 (5.6); 8.6027 (5.9); 8.5919 (6.3); 8.5864 (6.7); 8.5808 (12.0);
8.5715 (11.6); 8.0205 (4.4); 8.0166 (4.8); 7.9936 (4.7); 7.9906 (5.4); 7.7943 (3.4); 7.7885 (5.0); 7.7811 (3.4); 7.7680
(4.0); 7.7622 (5.6); 7.7549 (3.7); 7.6399 (3.9); 7.6356 (3.6); 7.6325 (3.1); 7.6147 (6.3); 7.6084 (6.2); 7.5621 (2.0);
7.5562 (2.7); 7.5392 (6.0); 7.5333 (4.6); 7.5291 (2.5); 7.5130 (9.8); 7.5057 (10.1); 7.4857 (16.0); 7.4799 (10.9);
7.4681 (5.0); 7.4625 (3.0); 7.4570 (5.9); 7.4394 (5.0); 7.3644 (4.8); 7.3619 (4.3); 7.3482 (4.7); 7.3457 (4.2); 7.3381
(4.4); 7.3357 (3.8); 7.3219 (4.1); 7.3195 (3.6); 7.2984 (11.2); 7.1981 (2.4); 7.1881 (3.8); 7.1717 (3.1); 7.1596 (7.5);
7.1567 (7.8); 7.1467 (3.6); 7.1333 (4.5); 7.1272 (6.8); 7.1175 (3.4); 5.7495 (7.0); 2.9841 (2.4); 2.9091 (2.1); 2.8083
(0.3); 2.1190 (2.3); 2.0512 (1.0); 2.0349 (13.0); 2.0182 (1.0); 1.2943 (0.5); 1.2878 (0.5); 1.2710 (0.5); 0.0433 (0.4);
0.0326 (12.4); 0.0216 (0.4)
I.002: 1H-NMR(300.2 MHz, CDCl3):
δ = 8.7126 (3.5); 8.5976 (2.1); 8.5815 (2.2); 8.5550 (1.9); 8.5271 (2.0); 7.8989 (1.6); 7.8718 (1.8); 7.8404 (1.6);
7.8128 (2.1); 7.6553 (0.8); 7.6286 (1.7); 7.6157 (0.8); 7.6052 (1.1); 7.5934 (1.0); 7.5879 (1.6); 7.5661 (1.5); 7.5602
(0.9); 7.5389 (1.9); 7.5160 (1.7); 7.4929 (0.8); 7.4886 (0.7); 7.4656 (1.3); 7.4478 (2.0); 7.4296 (1.2); 7.2984 (7.4);
7.0746 (1.2); 7.0446 (2.1); 7.0164 (1.1); 6.5274 (1.8); 2.4188 (16.0); 1.6383 (9.7); 1.2919 (0.4); 0.0364 (9.1)
I.003: 1H-NMR(400.2 MHz, d6-DMSO):
δ = 9.3181 (2.4); 9.3151 (2.9); 9.3123 (2.9); 9.3092 (2.5); 9.0425 (2.4); 9.0395 (2.4); 9.0291 (2.5); 9.0261 (2.5);
7.8267 (3.4); 7.7596 (2.2); 7.7536 (2.3); 7.7462 (2.2); 7.7402 (2.2); 7.5837 (0.8); 7.5798 (1.0); 7.5644 (1.6); 7.5604
(2.9); 7.5419 (2.4); 7.5381 (2.7); 7.4860 (0.5); 7.4692 (0.7); 7.4613 (1.6); 7.4481 (2.2); 7.4314 (1.0); 7.4232 (1.1);
7.4059 (1.1); 7.4005 (1.6); 7.3979 (1.7); 7.3819 (2.2); 7.3789 (2.4); 7.3724 (2.5); 7.3630 (1.0); 7.3601 (1.2); 7.3527
(2.0); 7.0810 (3.3); 7.0786 (3.3); 3.3490 (30.7); 2.8932 (1.2); 2.7354 (1.0); 2.7344 (1.1); 2.6266 (16.0); 2.5296 (0.5);
2.5161 (8.1); 2.5118 (15.9); 2.5073 (20.4); 2.5027 (14.9); 2.4983 (7.3); −0.0002 (1.3)
I.004: 1H-NMR(400.2 MHz, d6-DMSO):
δ = 8.3887 (2.8); 8.3842 (2.8); 8.1005 (2.6); 8.0969 (2.7); 7.9554 (0.5); 7.6707 (2.8); 7.6516 (3.4); 7.4955 (0.7);
7.4916 (0.8); 7.4765 (1.7); 7.4735 (1.6); 7.4574 (1.4); 7.4538 (1.8); 7.4399 (2.0); 7.4353 (2.1); 7.4309 (2.3); 7.4197
(3.7); 7.4165 (3.5); 7.4067 (1.1); 7.3981 (1.2); 7.3814 (1.0); 7.3580 (0.4); 7.3444 (2.1); 7.3393 (2.6); 7.3367 (2.2);
7.3274 (2.2); 7.3196 (1.9); 7.3097 (1.0); 6.9853 (3.3); 6.9830 (3.3); 3.3444 (9.9); 2.8923 (3.1); 2.7338 (2.7); 2.5824
(16.0); 2.5279 (0.6); 2.5143 (10.9); 2.5102 (21.0); 2.5057 (26.6); 2.5012 (19.3); 2.4970 (9.5); 2.1347 (13.0); −0.0002
(1.6)
I.005: 1H-NMR(400.2 MHz, d6-DMSO):
δ = 8.2102 (2.7); 8.1973 (2.8); 7.6589 (3.5); 7.5051 (0.8); 7.5013 (0.9); 7.4860 (1.7); 7.4828 (1.7); 7.4671 (1.2);
7.4633 (1.3); 7.4556 (0.5); 7.4367 (2.0); 7.4321 (4.1); 7.4162 (4.2); 7.4040 (1.3); 7.3876 (1.0); 7.3643 (0.4); 7.3304
(4.5); 7.3265 (4.6); 7.3116 (3.5); 7.2962 (1.0); 7.2099 (1.8); 7.2071 (1.8); 7.1970 (1.8); 7.1943 (1.7); 7.0345 (3.5);
3.3449 (20.6); 2.8924 (1.9); 2.7341 (1.7); 2.6067 (16.0); 2.5105 (19.5); 2.5061 (24.8); 2.5017 (18.3); 2.3020
(15.4); −0.0002 (1.3)
I.006: 1H-NMR(400.2 MHz, d6-DMSO):
δ = 8.3400 (5.2); 8.1306 (3.1); 8.1181 (3.2); 7.9554 (0.3); 7.5238 (0.6); 7.5204 (0.6); 7.5098 (0.8); 7.5062 (0.9);
7.4974 (2.1); 7.4895 (1.6); 7.4836 (1.8); 7.4800 (2.0); 7.4701 (4.8); 7.4629 (1.8); 7.4575 (1.6); 7.4553 (1.8); 7.4400
(1.1); 7.4322 (1.2); 7.4142 (1.0); 7.4093 (0.7); 7.3910 (0.5); 7.3079 (0.8); 7.2949 (10.5); 7.2888 (3.5); 7.2775 (3.2);
7.2617 (0.5); 7.2592 (0.4); 7.1344 (3.5); 7.1321 (3.6); 7.0650 (2.6); 7.0524 (2.6); 3.3512 (36.5); 2.8928 (2.4); 2.7342
(2.0); 2.5290 (0.6); 2.5156 (10.0); 2.5113 (19.6); 2.5067 (26.8); 2.5023 (32.7); 2.1772 (16.0); −0.0002 (1.3)
I.007: 1H-NMR(400.2 MHz, d6-DMSO):
δ = 8.5978 (5.0); 8.5938 (3.3); 8.5868 (3.2); 8.5828 (5.2); 7.9564 (0.3); 7.7924 (3.0); 7.7521 (0.6); 7.7481 (0.7);
7.7383 (0.8); 7.7340 (0.8); 7.7293 (1.0); 7.7250 (1.0); 7.7153 (0.9); 7.7117 (0.9); 7.6286 (0.8); 7.6103 (0.9); 7.6029
(1.0); 7.5848 (1.0); 7.5800 (0.7); 7.5616 (0.5); 7.4863 (0.7); 7.4712 (0.9); 7.4662 (1.4); 7.4514 (1.4); 7.4463 (1.1);
7.4313 (0.9); 7.3446 (4.3); 7.3414 (3.1); 7.3299 (4.2); 7.3150 (1.3); 7.2930 (2.0); 7.2716 (0.9); 7.0409 (2.1); 7.0216
(2.0); 6.6378 (0.4); 6.6169 (0.4); 4.0287 (6.7); 3.6173 (0.6); 3.5850 (2.4); 3.3563 (72.8); 2.8963 (2.2); 2.7382 (1.9);
2.7373 (1.9); 2.5179 (10.8); 2.5136 (21.2); 2.5091 (27.4); 2.5046 (20.3); 2.5003 (10.4); 2.4010 (16.0); −0.0002 (0.4)
I.008: 1H-NMR(400.2 MHz, d6-DMSO):
δ = 8.5556 (1.7); 8.5518 (1.8); 8.5436 (1.8); 8.5398 (1.8); 8.4795 (2.4); 8.4752 (2.5); 7.9561 (0.5); 7.7724 (3.1);
7.7474 (1.6); 7.7423 (1.8); 7.7381 (1.7); 7.7235 (2.4); 7.7175 (1.6); 7.6255 (0.8); 7.6072 (0.8); 7.6000 (1.0); 7.5819
(1.0); 7.5770 (0.7); 7.5584 (0.5); 7.4883 (0.7); 7.4733 (0.8); 7.4683 (1.4); 7.4534 (1.4); 7.4485 (1.1); 7.4334 (2.3);
7.4205 (1.4); 7.4141 (1.4); 7.4019 (1.2); 7.3159 (1.2); 7.2935 (1.9); 7.2719 (0.9); 7.0707 (2.2); 7.0516 (2.0); 4.0343
(6.8); 3.3635 (72.1); 3.3616 (74.1); 2.8965 (3.4); 2.7380 (3.0); 2.5315 (0.6); 2.5183 (9.9); 2.5142 (19.4); 2.5097
(25.1); 2.5053 (18.5); 2.3882 (16.0)
I.009: 1H-NMR(400.2 MHz, d6-DMSO):
δ = 9.6188 (0.6); 8.5487 (11.9); 8.3119 (0.8); 8.2839 (7.4); 8.2806 (8.1); 8.2721 (8.1); 8.2687 (7.9); 7.9543 (2.0);
7.8645 (0.5); 7.8455 (0.5); 7.8044 (6.4); 7.7848 (6.8); 7.6848 (14.7); 7.5502 (3.1); 7.5359 (3.9); 7.5303 (5.3); 7.5273
(5.3); 7.5165 (4.9); 7.4961 (3.6); 7.4757 (8.2); 7.4702 (5.4); 7.4592 (8.5); 7.4552 (7.4); 7.4440 (6.0); 7.4391 (5.9);
7.4265 (5.0); 7.4028 (2.5); 7.3790 (0.6); 7.3608 (0.3); 7.3468 (0.6); 7.3259 (1.1); 7.3051 (0.9); 7.2974 (0.6); 7.2718
(16.0); 7.2353 (6.3); 7.2232 (6.4); 7.2166 (6.4); 7.2044 (5.7); 7.1471 (5.3); 7.1253 (8.8); 7.1013 (6.4); 7.0959 (10.7);
7.0756 (9.3); 7.0444 (0.6); 7.0364 (0.6); 7.0261 (0.6); 7.0170 (0.8); 7.0063 (0.5); 6.9971 (0.4); 6.7082 (1.8); 6.6872
(1.7); 6.6372 (0.6); 6.6162 (0.6); 6.5550 (1.9); 6.1299 (2.5); 4.0122 (0.5); 3.6522 (0.6); 3.6155 (9.8); 3.5832 (3.3);
3.5565 (6.5); 3.3603 (499.3); 3.2019 (0.4); 2.9817 (0.3); 2.8941 (12.0); 2.7540 (0.4); 2.7356 (10.8); 2.7140 (0.6);
2.6955 (0.6); 2.6763 (0.9); 2.6334 (0.5); 2.5856 (1.0); 2.5746 (0.7); 2.5371 (70.5); 2.5116 (114.8); 2.5073 (145.5);
2.5030 (109.8); 2.3760 (0.5); 2.3602 (0.5); 2.3384 (0.8); 2.3340 (1.0); 1.7152 (0.4); 1.6458 (1.9); 1.6014 (1.9);
1.5534 (2.2); 1.4127 (0.7); 1.3784 (0.6); 1.3586 (0.5); 1.3390 (0.6); 1.2999 (0.9); 1.2597 (1.4); 1.2375 (5.7); 1.1856
(0.4); 1.1508 (0.4); 1.1143 (0.8); 1.0588 (3.3); 0.8698 (0.5); 0.8528 (1.1); 0.8353 (0.5); −0.0002 (2.2)
I.010: 1H-NMR(300.2 MHz, CDCl3):
δ = 8.7734 (2.6); 8.7543 (2.6); 7.7861 (2.9); 7.7833 (2.8); 7.4315 (0.6); 7.4133 (3.8); 7.3975 (4.4); 7.3888 (2.8);
7.3813 (2.7); 7.3684 (1.3); 7.3592 (2.4); 7.3362 (1.4); 7.2988 (4.1); 7.0433 (2.1); 7.0158 (1.8); 6.9478 (1.1); 6.9451
(1.0); 6.9173 (2.0); 6.8896 (1.0); 6.8868 (0.9); 5.4505 (1.9); 5.3340 (0.7); 2.5742 (16.0); 2.0791 (0.3); 1.7574 (0.6);
1.2886 (0.7); 0.0336 (4.4)
I.011: 1H-NMR(400.2 MHz, d6-DMSO):
δ = 9.1552 (7.0); 8.8135 (11.5); 7.9557 (0.4); 7.7797 (3.2); 7.7599 (0.7); 7.7560 (0.8); 7.7463 (0.8); 7.7421 (0.9);
7.7372 (1.0); 7.7331 (1.0); 7.7233 (0.9); 7.7197 (0.9); 7.6334 (0.8); 7.6152 (0.9); 7.6080 (1.0); 7.5898 (1.0); 7.5850
(0.7); 7.5665 (0.5); 7.5422 (0.7); 7.5271 (0.9); 7.5221 (1.4); 7.5071 (1.4); 7.5022 (1.1); 7.4871 (0.9); 7.3651 (1.2);
7.3432 (1.9); 7.3218 (1.0); 7.1072 (2.1); 7.0881 (2.0); 4.1000 (6.7); 3.3624 (66.4); 2.8969 (2.3); 2.7379 (2.1); 2.5146
(19.4); 2.5101 (25.1); 2.5056 (18.5); 2.5015 (9.5); 2.4289 (16.0)
I.012: 1H-NMR(300.2 MHz, CDCl3):
δ = 8.6104 (2.7); 8.6049 (2.8); 8.5373 (1.7); 8.5339 (1.8); 8.5210 (1.8); 8.5176 (1.8); 7.7579 (2.6); 7.7536 (2.6);
7.4654 (0.5); 7.4593 (0.8); 7.4379 (2.7); 7.4324 (3.1); 7.4198 (1.2); 7.4106 (1.4); 7.3987 (1.7); 7.3957 (1.6); 7.3825
(3.0); 7.3676 (0.8); 7.3616 (2.5); 7.3536 (1.4); 7.3325 (1.3); 7.2990 (7.6); 7.2810 (0.7); 7.2737 (0.7); 7.2678 (1.0);
7.2623 (1.0); 7.2433 (0.7); 7.2378 (0.7); 7.0277 (1.2); 7.0194 (1.5); 6.9929 (1.3); 6.9846 (1.5); 6.9560 (1.0); 6.9476
(0.8); 6.9291 (1.3); 6.9205 (1.1); 6.9013 (0.8); 6.8930 (0.7); 5.6154 (1.4); 4.1934 (0.3); 4.1695 (1.0); 4.1457 (1.0);
4.1219 (0.4); 2.6506 (16.0); 2.0820 (4.7); 1.6458 (5.5); 1.3197 (1.3); 1.3069 (0.8); 1.2959 (2.7); 1.2721 (1.2); 0.0471
(0.3); 0.0361 (9.7); 0.0254 (0.3)
I.013: 1H-NMR(400.2 MHz, d6-DMSO):
δ = 10.1518 (1.0); 10.1442 (1.0); 8.9091 (6.5); 8.8326 (9.6); 8.7558 (1.2); 8.6398 (1.9); 8.3893 (0.7); 8.3144 (0.6);
7.9546 (2.6); 7.8494 (3.4); 7.8392 (1.4); 7.8211 (0.4); 7.5869 (0.7); 7.5727 (0.9); 7.5672 (1.2); 7.5641 (1.2); 7.5505
(1.1); 7.5382 (0.8); 7.5177 (1.6); 7.5009 (2.4); 7.4808 (1.7); 7.4575 (1.1); 7.4338 (0.5); 7.3479 (3.7); 7.3320 (0.4);
7.3242 (0.6); 7.3117 (0.5); 7.3042 (0.4); 7.2916 (0.3); 7.2066 (0.5); 7.1865 (0.5); 7.1748 (1.4); 7.1525 (2.1); 7.1302
(1.1); 7.0911 (2.4); 7.0713 (2.4); 7.0552 (0.9); 3.6158 (0.8); 3.3787 (3.0); 3.2021 (0.4); 2.8948 (14.3); 2.7360 (13.1);
2.7208 (0.4); 2.5617 (16.0); 2.5111 (32.1); 2.5068 (41.0); 2.5026 (31.3); 2.4214 (5.0); 1.2603 (0.4); 1.2376
(1.4); −0.0002 (1.1)
I.014: 1H-NMR(300.2 MHz, CDCl3):
δ = 8.6400 (2.8); 8.6347 (2.8); 8.5484 (1.7); 8.5452 (1.8); 8.5322 (1.8); 8.5289 (1.8); 8.4373 (1.8); 8.4100 (1.9);
7.6434 (0.8); 7.6369 (0.8); 7.6139 (1.4); 7.5921 (0.8); 7.5858 (0.9); 7.5418 (0.6); 7.5369 (0.5); 7.5254 (0.6); 7.5195
(0.6); 7.5107 (1.2); 7.5057 (1.4); 7.4942 (1.2); 7.4884 (1.3); 7.4825 (1.3); 7.4579 (1.1); 7.4502 (1.4); 7.4254 (3.4);
7.4051 (3.9); 7.3987 (2.2); 7.3890 (1.7); 7.3836 (1.9); 7.3803 (1.7); 7.3566 (1.7); 7.3343 (0.6); 7.3309 (0.6); 7.2988
(17.6); 6.6741 (1.4); 5.3383 (0.7); 2.5466 (16.0); 1.6061 (13.7); 1.2908 (0.9); 0.0476 (0.7); 0.0368 (19.6); 0.0260
(0.8)
I.015: 1H-NMR(300.2 MHz, CDCl3):
δ = 8.7100 (3.0); 8.7064 (2.9); 8.6785 (0.4); 8.5958 (1.9); 8.5926 (2.0); 8.5797 (1.9); 8.5766 (1.8); 8.4866 (1.8);
8.4587 (1.9); 7.6224 (1.3); 7.6170 (0.9); 7.6018 (1.0); 7.5938 (3.2); 7.5887 (2.1); 7.5748 (2.4); 7.5676 (1.0); 7.5586
(1.1); 7.5508 (1.4); 7.5468 (1.1); 7.5327 (1.2); 7.5229 (0.5); 7.5048 (0.6); 7.4556 (1.2); 7.4374 (1.8); 7.4195 (1.2);
7.2989 (9.7); 7.2361 (0.9); 7.2305 (0.9); 7.2113 (0.8); 7.2028 (1.1); 7.1978 (1.0); 7.1768 (0.8); 7.1722 (0.8); 7.1060
(1.0); 7.1033 (1.0); 7.0757 (1.9); 7.0480 (0.9); 7.0453 (0.9); 6.6053 (1.5); 5.3374 (0.3); 2.4651 (16.0); 1.6210 (10.8);
0.0474 (0.4); 0.0366 (10.8); 0.0255 (0.4)
I.016: 1H-NMR(400.2 MHz, d6-DMSO):
δ = 9.2585 (2.4); 9.2558 (2.5); 9.2532 (2.4); 9.0715 (2.4); 9.0685 (2.3); 9.0582 (2.5); 9.0552 (2.4); 7.8762 (3.3);
7.7608 (1.3); 7.7591 (1.4); 7.7553 (1.4); 7.7476 (1.4); 7.7459 (1.4); 7.7421 (1.4); 7.5858 (0.6); 7.5821 (0.6); 7.5718
(0.7); 7.5633 (1.7); 7.5593 (1.2); 7.5469 (1.9); 7.5435 (2.2); 7.5266 (1.7); 7.5226 (1.1); 7.5066 (1.3); 7.4903 (0.9);
7.4827 (1.0); 7.4648 (0.9); 7.4598 (0.6); 7.4414 (0.5); 7.3681 (3.3); 7.1821 (1.2); 7.1603 (1.8); 7.1385 (1.0); 7.1370
(1.0); 7.1035 (2.3); 7.0832 (2.1); 3.3479 (29.8); 2.8938 (1.3); 2.7355 (1.1); 2.5773 (16.0); 2.5299 (0.4); 2.5249 (0.7);
2.5164 (8.2); 2.5120 (16.2); 2.5075 (20.8); 2.5029 (15.1); 2.4985 (7.3); −0.0002 (1.3)
I.017: 1H-NMR(400.2 MHz, d6-DMSO):
δ = 7.8962 (3.0); 7.7684 (0.6); 7.7642 (0.7); 7.7547 (0.7); 7.7503 (0.8); 7.7456 (1.0); 7.7414 (1.0); 7.7317 (0.9);
7.7279 (0.9); 7.6310 (0.7); 7.6127 (0.8); 7.6053 (1.0); 7.5872 (0.9); 7.5824 (0.7); 7.5640 (0.5); 7.3507 (0.7); 7.3358
(0.8); 7.3308 (1.4); 7.3161 (1.4); 7.3108 (1.1); 7.2960 (0.9); 7.1538 (1.1); 7.1320 (1.8); 7.1105 (0.9); 6.8627 (2.1);
6.8437 (1.9); 4.8253 (1.3); 4.8159 (2.8); 4.8065 (1.3); 4.2115 (6.7); 3.9355 (2.2); 3.9231 (2.8); 3.91 04 (2.3); 3.3679
(87.0); 2.8970 (1.8); 2.7386 (1.6); 2.5840 (16.0); 2.5324 (0.5); 2.5190 (8.4); 2.5147 (16.7); 2.5102 (21.9); 2.5057
(16.3); 2.5016 (8.4); 2.0684 (0.9); 2.0527 (2.3); 2.0430 (2.3); 2.0274 (1.1); 1.7725 (0.6); 1.7575 (1.7); 1.7439 (1.9);
1.7316 (1.5); 1.7161 (0.5)
I.018: 1H-NMR(400.2 MHz, d6-DMSO):
δ = 7.9548 (0.4); 7.7944 (3.3); 7.6758 (0.6); 7.6719 (0.7); 7.6622 (0.7); 7.6579 (0.8); 7.6527 (1.0); 7.6486 (1.1);
7.6391 (1.0); 7.6356 (1.0); 7.5677 (0.8); 7.5494 (0.8); 7.5424 (1.0); 7.5242 (1.0); 7.5006 (0.6); 7.4676 (3.3); 7.4402
(0.4); 7.4316 (0.4); 7.4242 (0.4); 7.3123 (0.8); 7.2958 (1.1); 7.2918 (1.6); 7.2756 (1.7); 7.2715 (1.1); 7.2550 (0.9);
6.9546 (2.3); 6.9341 (2.1); 6.8284 (1.2); 6.8055 (1.9); 6.7841 (1.1); 4.9802 (1.0); 4.9709 (2.1); 4.9624 (1.1); 3.8275
(2.0); 3.8149 (2.7); 3.8025 (2.2); 3.3695 (96.3); 3.2945 (0.4); 3.2811 (0.3); 2.8962 (2.7); 2.7376 (2.3); 2.6493 (16.0);
2.5310 (0.6); 2.5176 (10.9); 2.5134 (21.7); 2.5089 (28.4); 2.5045 (21.2); 1.9421 (0.9); 1.9267 (2.2); 1.9169 (2.2);
1.9014 (1.1); 1.5784 (0.6); 1.5640 (1.7); 1.5509 (2.0); 1.5380 (1.5); 1.5226 (0.6); 1.2602 (0.4); 1.2367 (1.1); −0.0002
(0.5)
I.019: 1H-NMR(400.2 MHz, d6-DMSO):
δ = 8.8225 (2.8); 8.8207 (3.0); 8.8171 (3.0); 8.8153 (2.7); 8.1147 (2.0); 8.1092 (1.9); 8.0945 (2.4); 8.0890 (2.3);
7.9565 (2.2); 7.8974 (3.0); 7.8957 (3.0); 7.8773 (2.5); 7.8755 (2.5); 7.7603 (3.6); 7.5597 (0.8); 7.5558 (0.9); 7.5404
(1.6); 7.5367 (2.0); 7.5177 (2.8); 7.5005 (2.3); 7.4971 (1.8); 7.4879 (0.6); 7.4712 (0.7); 7.4637 (1.9); 7.4547 (1.6);
7.4503 (1.6); 7.4390 (1.0); 7.4305 (1.1); 7.4137 (1.0); 7.4079 (0.4); 7.3854 (1.3); 7.3827 (1.5); 7.3667 (1.8); 7.3638
(2.6); 7.3585 (2.6); 7.3478 (1.0); 7.3450 (1.2); 7.3388 (2.0); 7.0863 (3.3); 7.0842 (3.3); 3.3492 (51.4); 2.8947 (14.9);
2.7363 (12.7); 2.7354 (12.4); 2.6017 (16.0); 2.5304 (0.5); 2.5170 (10.5); 2.5126 (20.5); 2.5080 (26.1); 2.5035 (18.7);
2.4990 (9.0); −0.0002 (0.9)
I.020: 1H-NMR(400.2 MHz, d6-DMSO):
δ = 8.8745 (3.8); 8.8692 (3.8); 8.7200 (4.0); 8.7150 (4.0); 8.4058 (2.4); 8.4007 (4.1); 8.3955 (2.2); 7.9558 (2.0);
7.7683 (3.6); 7.5496 (0.8); 7.5457 (0.9); 7.5303 (1.6); 7.5266 (2.3); 7.5184 (1.8); 7.5157 (1.8); 7.5120 (1.4); 7.5077
(1.9); 7.4996 (2.7); 7.4897 (0.8); 7.4864 (0.8); 7.4778 (1.4); 7.4635 (1.3); 7.4587 (1.2); 7.4412 (0.9); 7.4333 (1.1);
7.4158 (1.0); 7.4107 (0.5); 7.3925 (0.4); 7.3748 (1.2); 7.3722 (1.5); 7.3531 (4.4); 7.3329 (2.5); 7.0486 (3.4); 3.3444
(38.5); 2.8935 (13.6); 2.7352 (11.6); 2.7342 (11.8); 2.5992 (16.0); 2.5291 (0.6); 2.5155 (11.3); 2.5112 (21.9); 2.5068
(27.7); 2.5022 (20.0); 2.4978 (9.7); −0.0002 (1.4)
I.022: 1H-NMR(300.2 MHz, CDCl3):
δ = 8.6155 (0.7); 8.5984 (0.7); 8.4862 (1.2); 7.8102 (0.8); 7.4483 (0.4); 7.4202 (0.4); 7.3816 (0.5); 7.3683 (0.6);
7.3526 (0.8); 7.3387 (0.5); 7.3290 (1.1); 7.3124 (0.8); 7.2990 (7.5); 7.2821 (0.6); 7.2769 (0.6); 7.2075 (0.4); 7.1826
(0.5); 5.3380 (1.3); 5.2050 (0.6); 2.5630 (0.8); 2.5377 (0.9); 2.5127 (0.3); 2.4836 (4.3); 1.6097 (16.0); 1.1394 (1.2);
1.1142 (2.4); 1.0890 (1.1); 0.0478 (0.4); 0.0374 (9.2)
I.024: 1H-NMR(300.2 MHz, CDCl3):
δ = 8.6729 (0.6); 8.6670 (0.6); 8.6567 (0.6); 8.6509 (0.6); 7.8038 (1.1); 7.5755 (0.6); 7.5695 (0.6); 7.5500 (0.7);
7.5440 (0.7); 7.4397 (0.5); 7.4164 (0.5); 7.4120 (0.6); 7.3788 (0.6); 7.3639 (1.4); 7.3607 (1.3); 7.3478 (0.5); 7.3372
(1.0); 7.3165 (0.5); 7.2988 (8.0); 7.2793 (0.8); 7.2724 (1.0); 7.2546 (0.6); 7.2453 (0.6); 7.2292 (0.5); 7.2034 (0.5);
7.1994 (0.5); 7.1784 (0.6); 7.1755 (0.6); 5.1946 (0.7); 2.7733 (0.4); 2.7482 (1.4); 2.7231 (1.4); 2.6980 (0.5); 2.4783
(6.2); 1.6206 (16.0); 1.2912 (0.9); 1.2198 (1.8); 1.1947 (3.8); 1.1696 (1.7); 0.0370 (7.6)
I.025: 1H-NMR(400.2 MHz, d6-DMSO):
δ = 8.1728 (4.2); 7.6043 (0.3); 7.6005 (0.4); 7.5904 (0.4); 7.5865 (0.4); 7.5811 (0.6); 7.5775 (0.6); 7.5673 (0.6);
7.5640 (0.6); 7.5298 (0.5); 7.5119 (0.5); 7.5042 (0.6); 7.4862 (2.4); 7.4182 (0.5); 7.4136 (0.5); 7.3981 (0.8); 7.3959
(0.8); 7.3805 (0.7); 7.3757 (0.6); 7.1945 (0.4); 7.1920 (0.5); 7.1756 (1.1); 7.1734 (1.2); 7.1579 (1.0); 7.1555 (1.0);
7.1462 (1.5); 7.1417 (1.5); 7.1273 (0.6); 7.1228 (0.5); 7.1098 (1.3); 7.0901 (1.2); 7.0379 (1.8); 3.3454 (32.0); 2.8929
(0.8); 2.7343 (0.7); 2.7333 (0.7); 2.5280 (0.4); 2.5146 (8.0); 2.5102 (15.6); 2.5056 (19.8); 2.5011 (14.1); 2.4966
(6.7); 2.4607 (9.6); 2.0612 (16.0); −0.0002 (0.7)
I.027: 1H-NMR(400.2 MHz, d6-DMSO):
δ = 8.2285 (4.5); 8.0707 (2.8); 8.0589 (2.9); 7.4675 (0.8); 7.4634 (0.8); 7.4485 (3.4); 7.4437 (2.8); 7.4320 (3.7);
7.4255 (1.6); 7.4186 (1.1); 7.3392 (3.2); 7.3318 (1.8); 7.3161 (4.3); 7.2970 (1.6); 7.2473 (1.5); 7.2441 (1.4); 7.2395
(3.2); 7.2271 (7.2); 7.2103 (0.9); 7.2076 (0.8); 3.7944 (16.0); 3.3464 (24.5); 2.8926 (1.4); 2.7340 (1.2); 2.5498
(14.7); 2.5284 (0.5); 2.5148 (8.3); 2.5106 (16.3); 2.5061 (20.9); 2.5016 (15.4); 2.4974 (7.7); −0.0002 (0.8)
I.028: 1H-NMR(400.2 MHz, d6-DMSO):
δ = 7.9698 (2.3); 7.9689 (2.3); 7.9556 (3.4); 7.6350 (2.7); 7.5067 (0.6); 7.5027 (0.7); 7.4874 (1.4); 7.4851 (1.2);
7.4827 (1.1); 7.4684 (1.1); 7.4648 (1.3); 7.4520 (1.6); 7.4476 (1.8); 7.4430 (2.0); 7.4373 (1.6); 7.4312 (2.8); 7.4279
(2.8); 7.4135 (0.9); 7.3970 (0.8); 7.3325 (1.3); 7.3165 (3.5); 7.2970 (2.7); 7.0727 (2.6); 7.0703 (2.6); 7.0441 (1.8);
7.0406 (1.9); 7.0308 (1.8); 7.0273 (1.8); 6.8708 (3.1); 6.8692 (3.0); 3.7176 (16.0); 3.3429 (27.6); 2.8926 (7.9);
2.7346 (6.8); 2.7337 (6.6); 2.6039 (12.9); 2.5283 (0.5); 2.5149 (9.1); 2.5105 (17.8); 2.5060 (22.8); 2.5014 (16.4);
2.4970 (8.0); −0.0002 (1.0)
I.029: 1H-NMR(400.2 MHz, d6-DMSO):
δ = 9.0649 (2.7); 7.9577 (0.4); 7.8046 (1.4); 7.7854 (2.2); 7.7650 (1.9); 7.7570 (2.6); 7.7238 (1.4); 7.7203 (1.4);
7.7044 (1.5); 7.7008 (1.5); 7.5936 (0.5); 7.5900 (0.5); 7.5796 (0.6); 7.5760 (0.6); 7.5703 (0.9); 7.5670 (0.9); 7.5565
(0.8); 7.5535 (0.8); 7.5243 (0.7); 7.5063 (0.7); 7.4988 (0.8); 7.4809 (0.8); 7.4760 (0.5); 7.4575 (0.4); 7.4240 (0.6);
7.4203 (0.6); 7.4033 (1.4); 7.3900 (2.5); 7.3860 (1.3); 7.3820 (1.1); 7.3720 (2.2); 7.3243 (1.9); 7.3060 (1.2); 7.1665
(0.9); 7.1637 (0.9); 7.1473 (1.5); 7.1292 (0.8); 7.1263 (0.8); 6.7828 (2.4); 6.7625 (2.3); 3.9106 (16.0); 3.3489 (30.2);
2.8950 (2.9); 2.7368 (2.5); 2.6864 (12.4); 2.5309 (0.4); 2.5174 (7.9); 2.5132 (15.2); 2.5087 (19.3); 2.5042 (13.9);
2.4999 (6.8); −0.0002 (1.1)
I.030: 1H-NMR(400.2 MHz, d6-DMSO):
δ = 8.2476 (5.7); 8.2227 (3.0); 8.2084 (3.1); 7.4480 (3.3); 7.4368 (2.6); 7.4312 (3.3); 7.4180 (1.4); 7.4136 (2.0);
7.3308 (1.3); 7.3269 (1.4); 7.3112 (3.0); 7.3076 (3.9); 7.2903 (3.9); 7.2503 (1.3); 7.2473 (1.2); 7.2319 (1.8); 7.2290
(1.7); 7.2132 (0.8); 7.2103 (0.8); 7.1969 (2.9); 7.1939 (2.9); 6.9169 (2.9); 6.9024 (2.8); 3.7145 (16.0); 3.3490 (17.3);
2.8924 (1.3); 2.7344 (1.1); 2.7333 (1.1); 2.5222 (15.9); 2.5148 (10.2); 2.5103 (19.3); 2.5057 (24.9); 2.5012 (18.1);
2.4967 (8.9); −0.0002 (0.5)
I.031: 1H-NMR(400.2 MHz, d6-DMSO):
δ = 8.0391 (2.3); 7.9564 (0.6); 7.9144 (1.2); 7.9103 (1.1); 7.8900 (1.2); 7.8859 (1.1); 7.6599 (3.4); 7.4951 (0.8);
7.4912 (0.9); 7.4760 (2.2); 7.4569 (1.7); 7.4530 (3.1); 7.4421 (3.0); 7.4377 (3.0); 7.4269 (1.3); 7.4218 (2.9); 7.4184
(3.0); 7.4016 (1.0); 7.3958 (0.4); 7.3784 (0.4); 7.3323 (1.7); 7.3226 (2.2); 7.3195 (2.7); 7.3157 (2.5); 7.3027 (1.7);
7.2992 (2.1); 7.0164 (3.2); 7.0138 (3.2); 3.6152 (1.5); 3.3452 (35.3); 2.8937 (4.0); 2.7350 (3.4); 2.5964 (16.0);
2.5296 (0.5); 2.5162 (9.6); 2.5118 (19.1); 2.5073 (24.7); 2.5027 (17.9); 2.4983 (8.7); 2.0841 (10.6); −0.0002 (1.6)
I.032: 1H-NMR(400.2 MHz, d6-DMSO):
δ = 8.3006 (2.8); 8.0903 (2.8); 7.9553 (2.0); 7.6755 (4.3); 7.6125 (3.2); 7.5743 (0.8); 7.5704 (0.8); 7.5605 (0.9);
7.5564 (1.0); 7.5512 (1.3); 7.5473 (1.4); 7.5373 (1.2); 7.5338 (1.2); 7.4868 (1.1); 7.4777 (1.2); 7.4686 (1.2); 7.4612
(2.5); 7.4574 (2.5); 7.4412 (3.0); 7.4372 (2.1); 7.4203 (1.7); 7.2891 (4.1); 7.1198 (1.4); 7.1178 (1.5); 7.0966 (2.5);
7.0943 (2.0); 7.0756 (4.0); 7.0558 (2.6); 3.3460 (28.6); 2.8927 (14.0); 2.7345 (11.7); 2.7334 (11.7); 2.5290 (22.1);
2.5149 (14.8); 2.5105 (29.1); 2.5059 (37.5); 2.5013 (27.2); 2.4968 (13.3); 2.1076 (16.0); −0.0002 (1.7)
I.033: 1H-NMR(400.2 MHz, d6-DMSO):
δ = 8.2223 (2.6); 8.2097 (2.7); 7.9543 (0.3); 7.6563 (3.4); 7.5755 (0.6); 7.5717 (0.6); 7.5616 (0.7); 7.5576 (0.8);
7.5524 (1.1); 7.5486 (1.1); 7.5384 (1.0); 7.5351 (1.0); 7.4929 (0.9); 7.4753 (1.7); 7.4672 (1.1); 7.4591 (1.3); 7.4550
(2.1); 7.4438 (0.9); 7.4388 (1.9); 7.4347 (1.2); 7.4260 (0.6); 7.4183 (1.0); 7.3277 (3.3); 7.2536 (3.2); 7.1385 (1.8);
7.1257 (1.7); 7.0943 (1.3); 7.0728 (1.9); 7.0537 (2.6); 7.0341 (2.1); 3.6132 (0.4); 3.3395 (47.9); 2.8921 (2.4); 2.7336
(2.0); 2.7326 (2.0); 2.5453 (17.5); 2.5271 (1.3); 2.5222 (1.7); 2.5136 (17.3); 2.5092 (33.9); 2.5047 (43.4); 2.5001
(31.0); 2.4956 (14.8); 2.2710 (16.0); −0.0002 (2.3)
I.034: 1H-NMR(400.2 MHz, d6-DMSO):
δ = 8.4287 (5.6); 8.2937 (3.3); 8.2813 (3.5); 7.9555 (1.8); 7.6631 (3.1); 7.5346 (0.8); 7.5304 (0.8); 7.5143 (1.5);
7.5123 (1.6); 7.4964 (1.2); 7.4921 (1.4); 7.4675 (3.8); 7.4519 (2.5); 7.4448 (4.1); 7.4321 (3.4); 7.4064 (0.3); 7.3619
(1.3); 7.3580 (1.4); 7.3508 (0.3); 7.3429 (2.3); 7.3390 (2.1); 7.3327 (0.4); 7.3300 (0.4); 7.3124 (2.5); 7.2984 (1.9);
7.2952 (2.6); 7.2801 (2.1); 7.2778 (1.8); 7.2614 (0.8); 7.2588 (0.8); 7.2060 (0.3); 7.1620 (3.1); 7.1598 (3.1); 3.3444
(35.1); 2.8929 (12.5); 2.7348 (10.4); 2.7339 (10.6); 2.6997 (1.4); 2.5650 (16.0); 2.5286 (0.6); 2.5151 (10.7); 2.5108
(21.2); 2.5063 (27.2); 2.5017 (19.7); 2.4973 (9.6); −0.0002 (1.8)
I.035: 1H-NMR(400.2 MHz, d6-DMSO):
δ = 9.6057 (3.3); 7.9848 (1.3); 7.9652 (3.5); 7.9457 (2.6); 7.9109 (3.2); 7.8925 (1.8); 7.8615 (3.3); 7.8320 (1.8);
7.8285 (1.8); 7.8123 (2.0); 7.8088 (1.9); 7.6126 (0.6); 7.6092 (0.7); 7.5990 (0.7); 7.5950 (0.8); 7.5895 (1.2); 7.5854
(1.4); 7.5786 (2.2); 7.5606 (2.5); 7.5355 (0.9); 7.5174 (1.0); 7.5099 (1.1); 7.4957 (4.4); 7.4769 (4.1); 7.4569 (0.9);
7.4534 (0.8); 7.1966 (1.2); 7.1940 (1.2); 7.1764 (2.0); 7.1589 (1.0); 7.1563 (1.0); 3.3474 (51.6); 2.8956 (1.4); 2.7889
(16.0); 2.7365 (1.2); 2.6737 (0.4); 2.5312 (0.8); 2.5177 (13.2); 2.5134 (25.9); 2.5090 (33.4); 2.5044 (24.4); 2.5001
(12.2); −0.0002 (1.9)
I.036: 1H-NMR(400.2 MHz, d6-DMSO):
δ = 8.1908 (2.8); 8.1786 (2.9); 8.1779 (3.0); 7.9555 (1.6); 7.7649 (3.4); 7.5870 (3.6); 7.5850 (3.7); 7.5460 (0.8);
7.5421 (0.9); 7.5268 (1.7); 7.5238 (1.6); 7.5079 (1.1); 7.5039 (1.4); 7.4935 (1.7); 7.4892 (1.8); 7.4842 (0.9); 7.4732
(2.9); 7.4699 (2.5); 7.4613 (1.7); 7.4500 (4.2); 7.4467 (3.9); 7.4373 (2.6); 7.4336 (3.0); 7.4244 (1.1); 7.4073 (1.0);
7.4015 (0.5); 7.3840 (0.4); 7.3519 (1.9); 7.3450 (2.4); 7.3421 (2.3); 7.3348 (2.4); 7.3250 (1.9); 7.3223 (1.8); 7.3169
(1.3); 7.3140 (0.8); 7.0793 (3.1); 7.0768 (3.2); 3.3641 (0.6); 3.3606 (0.6); 3.3450 (45.8); 3.3316 (0.6); 2.9038 (0.4);
2.8932 (11.3); 2.7449 (0.3); 2.7349 (9.5); 2.6997 (1.0); 2.6240 (16.0); 2.5154 (12.4); 2.5110 (24.0); 2.5064 (30.5);
2.5018 (21.6); 2.4974 (10.1); 1.2309 (0.4); −0.0002 (1.5)
I.037: 1H-NMR(400.2 MHz, d6-DMSO):
δ = 8.4662 (3.1); 8.4611 (3.0); 8.4600 (3.0); 7.9554 (0.4); 7.9303 (2.0); 7.9240 (1.9); 7.9096 (2.2); 7.9033 (2.1);
7.6899 (3.5); 7.5197 (0.8); 7.5158 (0.9); 7.5004 (1.6); 7.4967 (2.0); 7.4772 (2.6); 7.4719 (1.9); 7.4560 (3.9); 7.4439
(1.9); 7.4288 (1.1); 7.4203 (1.2); 7.4035 (1.0); 7.3802 (0.4); 7.3641 (4.3); 7.3433 (5.2); 7.3338 (2.6); 7.3243 (1.2);
7.3142 (2.0); 7.0298 (3.4); 3.6145 (0.3); 3.3439 (45.4); 2.8930 (2.8); 2.7339 (2.4); 2.6022 (16.0); 2.5288 (0.8);
2.5151 (12.9); 2.5109 (24.9); 2.5064 (31.8); 2.5019 (23.2); 2.4976 (11.5); −0.0002 (1.2)
I.038: 1H-NMR(400.2 MHz, d6-DMSO):
δ = 8.1161 (2.6); 8.1113 (2.9); 8.1043 (2.9); 8.0994 (2.8); 7.9551 (2.6); 7.8564 (2.6); 7.8516 (2.6); 7.8374 (2.9);
7.8326 (2.7); 7.6194 (4.4); 7.5061 (1.1); 7.5022 (1.2); 7.4865 (2.3); 7.4838 (2.4); 7.4680 (5.7); 7.4527 (3.6); 7.4432
(1.6); 7.4273 (1.4); 7.4043 (0.6); 7.3862 (1.6); 7.3824 (2.2); 7.3658 (2.8); 7.3626 (2.7); 7.2919 (6.3); 7.2727 (6.3);
7.2557 (1.3); 7.2530 (1.1); 7.2409 (2.8); 7.2290 (2.7); 7.2219 (2.6); 7.2101 (2.6); 7.1571 (4.4); 3.6136 (0.5); 3.3425
(44.6); 2.8924 (16.0); 2.7343 (13.6); 2.7332 (14.1); 2.6993 (0.6); 2.6700 (0.4); 2.5479 (21.0); 2.5279 (0.9); 2.5101
(29.9); 2.5056 (38.2); 2.5011 (28.2); 2.3162 (0.6); −0.0002 (2.3)
I.040: 1H-NMR(400.2 MHz, d6-DMSO):
δ = 8.5456 (3.7); 8.5411 (3.8); 8.3180 (3.6); 8.3122 (3.8); 8.0042 (2.1); 7.9989 (3.4); 7.9938 (2.1); 7.9543 (2.2);
7.7465 (3.3); 7.5345 (0.7); 7.5307 (0.9); 7.5151 (1.6); 7.5130 (1.6); 7.5109 (1.6); 7.5026 (1.6); 7.4978 (2.1); 7.4929
(1.8); 7.4821 (3.0); 7.4696 (0.7); 7.4586 (1.4); 7.4432 (1.9); 7.4255 (0.9); 7.4174 (1.0); 7.4001 (0.9); 7.3767 (0.4);
7.3648 (1.6); 7.3484 (4.3); 7.3294 (3.2); 7.0246 (3.3); 7.0224 (3.3); 3.3425 (55.9); 2.8918 (14.7); 2.7323 (12.8);
2.5970 (16.0); 2.5271 (0.9); 2.5135 (17.8); 2.5093 (35.3); 2.5048 (45.8); 2.5003 (33.8); 2.4960 (17.0); 1.2322
(0.4); −0.0002 (0.4)
I.041: 1H-NMR(300.2 MHz, CDCl3):
δ = 8.5732 (2.3); 8.4926 (1.4); 8.4769 (1.4); 7.5981 (2.6); 7.4765 (0.5); 7.4714 (0.5); 7.4600 (1.2); 7.4533 (0.6);
7.4460 (1.1); 7.4410 (1.8); 7.4330 (1.6); 7.4231 (1.1); 7.4144 (1.4); 7.4066 (2.1); 7.3869 (1.4); 7.3847 (1.4); 7.3746
(1.3); 7.3521 (1.2); 7.3440 (0.6); 7.3214 (0.6); 7.2984 (4.0); 7.2283 (1.1); 7.2106 (2.5); 7.1919 (1.2); 7.1834 (1.8);
7.1556 (0.8); 6.9534 (1.8); 6.9277 (1.6); 4.0768 (0.9); 4.0213 (2.4); 3.9590 (2.4); 3.9035 (0.9); 2.5397 (16.0); 2.0100
(0.4); 1.7260 (2.4); 1.2974 (0.6); 1.2926 (0.6); 1.2765 (5.2); 0.9141 (0.6); 0.0330 (2.9)
I.042: 1H-NMR(400.2 MHz, d6-DMSO):
δ = 8.1870 (2.5); 8.1823 (2.6); 8.1138 (1.2); 7.9667 (0.8); 7.9600 (1.1); 7.9463 (1.5); 7.9405 (1.4); 7.9260 (0.8);
7.9199 (0.8); 7.8055 (0.3); 7.8006 (0.4); 7.7821 (3.6); 7.7690 (0.6); 7.7646 (0.5); 7.7553 (0.7); 7.7514 (0.8); 7.7413
(0.8); 7.7371 (0.9); 7.7324 (1.1); 7.7287 (1.1); 7.7187 (1.0); 7.6336 (1.1); 7.6152 (1.3); 7.6091 (1.3); 7.5908 (1.2);
7.5666 (0.5); 7.4944 (0.7); 7.4791 (1.0); 7.4747 (1.5); 7.4596 (1.6); 7.4546 (1.2); 7.4394 (0.9); 7.3849 (0.4); 7.3673
(0.6); 7.3476 (0.4); 7.3206 (1.2); 7.2976 (1.9); 7.2763 (1.0); 7.2208 (1.5); 7.2142 (1.5); 7.1998 (1.5); 7.1930 (1.4);
7.0940 (0.4); 7.0761 (1.4); 7.0653 (2.6); 7.0467 (2.7); 6.7082 (0.6); 6.6871 (0.5); 4.2165 (2.9); 4.0547 (6.9); 3.6179
(3.0); 3.3448 (33.3); 3.3414 (38.2); 2.8971 (4.0); 2.7388 (3.6); 2.6098 (6.5); 2.5135 (27.1); 2.5092 (33.7); 2.5048
(24.7); 2.4160 (16.0); 1.6101 (0.3); 1.5566 (0.3); 1.2357 (0.6); 1.0547 (0.5); −0.0002 (0.6)
I.043: 1H-NMR(400.2 MHz, d6-DMSO):
δ = 8.2446 (1.6); 8.2325 (1.6); 8.0248 (0.9); 8.0202 (0.9); 8.0064 (1.1); 8.0011 (1.6); 7.9959 (1.0); 7.9820 (1.0);
7.9773 (0.9); 7.9563 (0.8); 7.7549 (3.3); 7.7111 (0.7); 7.7074 (0.7); 7.6971 (0.8); 7.6933 (0.9); 7.6881 (1.1); 7.6845
(1.2); 7.6743 (1.0); 7.6710 (1.0); 7.6262 (0.8); 7.6082 (0.9); 7.6006 (1.1); 7.5828 (1.0); 7.5779 (0.7); 7.5595 (0.5);
7.5317 (0.7); 7.5164 (0.9); 7.5117 (1.4); 7.4965 (1.5); 7.4918 (1.2); 7.4765 (1.0); 7.4330 (1.0); 7.4282 (1.1); 7.4208
(1.1); 7.4153 (1.7); 7.4099 (1.1); 7.4025 (1.0); 7.3976 (1.0); 7.3315 (1.2); 7.3095 (2.0); 7.2876 (1.0); 7.1089 (2.3);
7.0897 (2.1); 4.1139 (1.1); 4.0723 (2.7); 4.0254 (2.7); 3.9838 (1.0); 3.6170 (1.5); 3.3455 (51.1); 2.8960 (4.7); 2.7375
(4.3); 2.5128 (20.8); 2.5085 (26.8); 2.5042 (20.1); 2.3931 (16.0); 1.2363 (0.3); −0.0002 (0.4)
I.044: 1H-NMR(400.2 MHz, d6-DMSO):
δ = 8.3120 (1.4); 8.2552 (2.6); 8.2425 (2.7); 7.9539 (0.4); 7.8131 (3.3); 7.7537 (0.7); 7.7397 (0.9); 7.7349 (1.1);
7.7310 (1.0); 7.7207 (1.0); 7.7179 (1.0); 7.6360 (0.8); 7.6174 (0.9); 7.6107 (1.0); 7.5928 (1.0); 7.5691 (0.5); 7.5059
(0.7); 7.4907 (0.8); 7.4858 (1.4); 7.4707 (1.4); 7.4661 (1.1); 7.4508 (0.9); 7.3302 (1.7); 7.3242 (1.8); 7.3103 (3.3);
7.2873 (1.0); 7.2428 (3.2); 7.0395 (2.2); 7.0204 (2.1); 4.0668 (7.0); 3.3375 (111.7); 2.8946 (2.4); 2.7356 (2.2);
2.6747 (0.4); 2.5099 (48.8); 2.5057 (62.6); 2.5015 (47.4); 2.4277 (16.0); 2.3326 (0.4); 1.2383 (1.1); −0.0002 (2.1)
I.045: 1H-NMR(400.2 MHz, d6-DMSO):
δ = 8.3137 (0.5); 8.2394 (2.6); 8.0610 (5.1); 8.0247 (0.7); 8.0192 (0.8); 8.0043 (1.5); 7.9989 (1.4); 7.9839 (0.8);
7.9782 (0.8); 7.9560 (2.3); 7.7171 (0.8); 7.7129 (0.9); 7.6973 (4.4); 7.6806 (1.0); 7.6765 (0.9); 7.5786 (1.0); 7.5731
(0.9); 7.5610 (1.6); 7.5537 (2.1); 7.5363 (2.0); 7.5124 (0.6); 7.4913 (1.2); 7.4745 (1.9); 7.4703 (2.7); 7.4537 (2.5);
7.4502 (2.3); 7.4335 (1.1); 7.4269 (0.6); 7.3393 (0.8); 7.3255 (3.9); 7.3194 (2.3); 7.3014 (1.7); 7.2810 (0.8); 7.1327
(1.3); 7.1112 (2.2); 7.0898 (1.2); 7.0596 (4.0); 7.0540 (2.2); 7.0390 (3.8); 7.0325 (1.8); 6.9797 (1.5); 6.9761 (1.6);
6.9592 (1.2); 6.9558 (1.5); 6.9293 (0.9); 6.9237 (1.5); 6.9183 (0.8); 6.9004 (0.9); 6.8948 (1.5); 6.8893 (0.8); 6.7333
(0.7); 6.7283 (0.7); 6.7128 (1.3); 6.7069 (1.2); 6.6923 (0.7); 6.6862 (0.6); 3.5119 (0.7); 3.3432 (44.5); 3.3395 (52.9);
2.8959 (15.3); 2.7377 (13.4); 2.7150 (0.4); 2.6883 (16.0); 2.5568 (17.6); 2.5167 (18.4); 2.5125 (36.8); 2.5080 (48.1);
2.5035 (36.0); 1.2612 (0.4); 1.2360 (2.0); 0.8526 (0.4); −0.0002 (1.6)
I.046: 1H-NMR(400.2 MHz, d6-DMSO):
δ = 8.0642 (4.0); 8.0517 (2.9); 7.9550 (1.1); 7.7475 (3.1); 7.6940 (0.3); 7.6894 (0.3); 7.6759 (0.3); 7.5790 (0.8);
7.5549 (1.4); 7.5411 (1.1); 7.5117 (0.9); 7.5035 (0.9); 7.4912 (1.7); 7.4858 (1.2); 7.4752 (2.1); 7.4600 (1.2); 7.4546
(1.4); 7.4367 (0.6); 7.3896 (3.4); 7.3550 (1.5); 7.3420 (1.6); 7.3195 (0.6); 7.3018 (0.6); 7.2400 (3.1); 7.1186 (1.1);
7.0952 (1.8); 7.0735 (1.1); 7.0531 (2.3); 7.0329 (2.1); 6.9788 (0.5); 6.9752 (0.5); 6.9584 (0.4); 6.9549 (0.5); 6.9223
(0.5); 6.8934 (0.5); 6.7132 (0.4); 6.7082 (0.4); 3.6561 (0.5); 3.6174 (0.4); 3.3711 (103.7); 3.3684 (101.5); 2.8962
(7.1); 2.7375 (6.1); 2.6876 (5.3); 2.5835 (1.1); 2.5724 (16.0); 2.5315 (0.7); 2.5180 (13.7); 2.5137 (27.7); 2.5093
(36.5); 2.5048 (27.4); 1.2610 (0.4); 1.2365 (1.4); −0.0002 (0.6)
I.047: 1H-NMR(400.2 MHz, d6-DMSO):
δ = 8.3100 (0.7); 8.0336 (7.6); 8.0241 (7.2); 8.0216 (7.6); 7.9765 (4.0); 7.9718 (3.9); 7.9577 (5.7); 7.9530 (8.5);
7.9481 (4.7); 7.9340 (4.4); 7.9293 (3.9); 7.7603 (16.0); 7.5471 (2.4); 7.5307 (3.2); 7.5229 (8.5); 7.5091 (11.8);
7.4956 (6.0); 7.4881 (12.9); 7.4714 (12.2); 7.4509 (4.8); 7.3619 (16.0); 7.2396 (4.4); 7.2349 (4.8); 7.2275 (4.8);
7.2219 (7.5); 7.2164 (4.9); 7.2090 (4.5); 7.2042 (4.4); 7.0902 (5.6); 7.0683 (9.6); 7.0464 (5.2); 7.0251 (11.1); 7.0049
(10.4); 4.0131 (0.4); 3.6171 (0.4); 3.4464 (0.4); 3.3700 (412.0); 3.3679 (399.6); 2.8956 (9.0); 2.7371 (7.9); 2.7158
(0.5); 2.6991 (0.3); 2.6923 (0.5); 2.6872 (0.4); 2.6825 (0.6); 2.6778 (0.8); 2.6736 (0.6); 2.6424 (0.6); 2.6346 (0.3);
2.5980 (1.1); 2.5757 (0.6); 2.5345 (79.1); 2.5175 (46.4); 2.5133 (90.4); 2.5089 (117.5); 2.5045 (87.6); 2.3731 (0.5);
2.3402 (0.5); 2.3358 (0.7); 2.3313 (0.6); 1.3012 (0.6); 1.2606 (1.1); 1.2369 (4.2); 0.8694 (0.3); 0.8528 (0.9); 0.8348
(0.4); −0.0002 (2.3)
I.049: 1H-NMR(300.1 MHz, d6-DMSO):
δ = 8.4146 (9.2); 8.4085 (9.4); 8.2588 (6.0); 8.2558 (6.1); 8.2425 (6.3); 8.2395 (6.2); 7.6529 (10.4); 7.4852 (5.4);
7.4684 (6.2); 7.4639 (7.7); 7.4541 (12.6); 7.4482 (7.7); 7.4289 (9.2); 7.4208 (9.3); 7.4135 (8.9); 7.4040 (10.4);
7.3955 (16.0); 7.3697 (5.9); 7.0552 (9.8); 7.0517 (9.4); 3.3507 (19.3); 2.9873 (0.7); 2.5510 (49.1); 2.5203 (6.8);
2.5144 (12.7); 2.5084 (16.7); 2.5025 (11.5); 2.4967 (5.2); 2.0808 (10.7); −0.0004 (0.4)
I.050: 1H-NMR(300.2 MHz, CDCl3):
δ = 8.5830 (0.4); 7.2988 (3.9); 4.3044 (0.4); 4.2338 (0.5); 4.1671 (0.4); 2.5514 (2.1); 1.9731 (0.4); 1.6254 (6.0);
1.3082 (16.0); 1.2900 (0.4); 1.2791 (6.8); 0.0360 (3.9)
I.051: 1H-NMR(300.2 MHz, d6-DMSO):
δ = 8.4611 (10.9); 8.4569 (11.0); 8.3125 (7.1); 8.2980 (7.0); 7.8066 (10.1); 7.5834 (2.5); 7.5522 (10.7); 7.5428 (7.5);
7.5302 (14.8); 7.5103 (12.8); 7.5036 (9.2); 7.4979 (6.9); 7.4922 (8.1); 7.4760 (4.1); 7.4571 (2.1); 7.4321 (11.8);
7.2975 (0.6); 7.1123 (5.2); 7.0836 (8.0); 7.0527 (5.1); 7.0388 (8.7); 7.0117 (7.7); 6.9570 (0.8); 6.9349 (0.6); 6.9137
(0.6); 6.8749 (0.6); 6.8160 (0.5); 6.8033 (0.5); 6.7709 (0.5); 6.7390 (0.5); 6.6697 (0.5); 6.5754 (0.4); 5.7648 (0.8);
2.7651 (0.4); 2.5544 (58.2); 2.5244 (8.4); 2.5187 (15.8); 2.5127 (20.9); 2.5068 (15.3); 2.3399 (0.3); 2.0841 (16.0);
1.2286 (0.6); −0.0003 (1.2)
I.052: 1H-NMR(400.2 MHz, d6-DMSO):
δ = 8.0632 (2.6); 7.9872 (1.1); 7.9797 (2.5); 7.9762 (2.4); 7.9562 (3.0); 7.7391 (2.5); 7.7122 (2.2); 7.5525 (0.6);
7.5487 (0.6); 7.5332 (1.7); 7.5304 (1.5); 7.5211 (1.1); 7.5100 (2.2); 7.5064 (2.0); 7.5014 (1.8); 7.4940 (2.7); 7.4862
(1.7); 7.4830 (1.7); 7.4779 (1.8); 7.4696 (1.5); 7.4610 (0.9); 7.4457 (2.6); 7.4264 (2.4); 7.3235 (1.1); 7.3174 (1.2);
7.3120 (1.3); 7.3058 (1.2); 7.2957 (3.8); 7.2920 (3.7); 7.2758 (4.0); 7.2726 (3.6); 7.2604 (3.3); 7.1956 (2.1); 3.3500
(73.8); 2.8940 (16.0); 2.7356 (13.9); 2.7346 (13.6); 2.7003 (0.4); 2.5822 (12.0); 2.5731 (10.4); 2.5293 (0.7); 2.5158
(13.9); 2.5117 (26.6); 2.5072 (33.7); 2.5027 (24.6); 2.4987 (12.3); −0.0002 (1.0)
I.053: 1H-NMR(400.2 MHz, d6-DMSO):
δ = 7.9557 (2.0); 7.8635 (2.0); 7.8612 (2.0); 7.8509 (2.1); 7.8485 (2.0); 7.7057 (3.4); 7.5475 (0.8); 7.5436 (0.9);
7.5282 (2.0); 7.5253 (1.7); 7.5236 (1.6); 7.5183 (0.8); 7.5153 (0.8); 7.5087 (2.8); 7.4990 (1.6); 7.4949 (1.6); 7.4832
(1.1); 7.4747 (1.3); 7.4596 (2.1); 7.4517 (0.8); 7.4429 (1.9); 7.3976 (1.8); 7.3855 (2.9); 7.3733 (1.7); 7.2959 (3.3);
7.2885 (2.8); 7.2861 (3.0); 7.2834 (2.2); 7.2698 (2.5); 7.2660 (2.3); 7.2633 (1.8); 7.2533 (1.2); 7.2505 (0.9); 3.6148
(1.6); 3.3440 (38.1); 2.8934 (13.6); 2.7351 (11.4); 2.7340 (11.6); 2.5736 (16.0); 2.5288 (0.7); 2.5153 (11.9); 2.5109
(23.4); 2.5064 (29.9); 2.5018 (21.6); 2.4973 (10.4); −0.0002 (1.8)
I.054: 1H-NMR(400.2 MHz, d6-DMSO):
δ = 7.9555 (0.9); 7.7790 (3.5); 7.5553 (0.8); 7.5516 (1.0); 7.5359 (1.8); 7.5333 (1.8); 7.5195 (2.3); 7.5156 (2.8);
7.4995 (3.1); 7.4965 (2.6); 7.4893 (2.3); 7.4767 (2.1); 7.4634 (1.4); 7.4549 (1.3); 7.4378 (1.0); 7.4240 (0.4); 7.4148
(0.5); 7.3344 (1.7); 7.3258 (2.4); 7.3231 (2.6); 7.3171 (2.6); 7.3057 (2.0); 7.3033 (2.1); 7.2994 (1.6); 7.2438 (14.8);
7.1739 (3.4); 3.3373 (40.2); 2.8925 (5.8); 2.7331 (5.1); 2.6318 (16.0); 2.5598 (1.0); 2.5098 (35.4); 2.5054 (45.2);
2.5009 (33.9); 1.2320 (0.4)
I.055: 1H-NMR(400.2 MHz, d6-DMSO):
δ = 8.1542 (0.6); 8.1338 (1.5); 8.1102 (1.4); 8.0898 (0.6); 7.9554 (2.0); 7.6308 (3.2); 7.5135 (0.5); 7.5077 (0.9);
7.5038 (1.0); 7.4981 (0.7); 7.4892 (3.9); 7.4735 (2.3); 7.4698 (1.8); 7.4651 (1.9); 7.4471 (1.0); 7.4404 (0.3); 7.4242
(1.6); 7.4072 (1.8); 7.2879 (1.1); 7.2853 (1.6); 7.2745 (2.1); 7.2715 (2.8); 7.2689 (2.6); 7.2544 (1.7); 7.2510 (2.6);
7.1963 (3.0); 7.0593 (1.4); 7.0538 (1.6); 7.0391 (1.4); 7.0335 (1.5); 3.3380 (30.4); 2.8926 (14.2); 2.7339 (11.9);
2.7331 (11.7); 2.5601 (16.0); 2.5276 (0.6); 2.5142 (13.3); 2.5098 (26.6); 2.5053 (34.3); 2.5007 (24.6); 2.4962 (11.9);
1.2325 (0.3)
I.056: 1H-NMR(300.2 MHz, CDCl3):
δ = 8.7057 (1.4); 8.7021 (1.4); 8.5933 (0.8); 8.5901 (0.8); 8.5771 (0.8); 8.5739 (0.8); 8.4209 (0.8); 8.3931 (0.9);
7.6203 (0.3); 7.5924 (0.8); 7.5790 (0.3); 7.5713 (0.8); 7.5644 (0.9); 7.5585 (0.6); 7.5479 (0.5); 7.5423 (0.8); 7.5210
(0.5); 7.4959 (0.4); 7.4886 (0.6); 7.4635 (0.5); 7.4485 (0.6); 7.4310 (1.0); 7.4124 (0.5); 7.2989 (10.4); 7.1134 (0.5);
7.1106 (0.5); 7.0829 (0.9); 7.0552 (0.4); 7.0527 (0.4); 6.5837 (0.7); 2.4764 (7.0); 2.0483 (0.6); 1.6070 (16.0); 0.0362
(9.1); 0.0251 (0.4)
I.057: 1H-NMR(300.2 MHz, CDCl3):
δ = 8.5236 (0.6); 8.5059 (0.6); 8.4569 (1.0); 7.7653 (0.6); 7.7615 (0.6); 7.4262 (0.6); 7.4176 (0.4); 7.3977 (0.5);
7.3925 (0.4); 7.3522 (0.7); 7.3318 (0.6); 7.3210 (0.3); 7.2988 (8.6); 7.2401 (0.3); 7.2358 (0.3); 6.7358 (0.5); 6.7179
(0.5); 5.4059 (0.4); 5.3378 (0.6); 2.5131 (3.5); 1.6113 (16.0); 1.2913 (0.6); 0.0370 (7.7)
I.059: 1H-NMR(300.2 MHz, CDCl3):
δ = 8.5290 (1.5); 8.5232 (1.6); 8.5131 (1.6); 8.5073 (1.6); 7.7439 (2.6); 7.7396 (2.7); 7.5350 (1.8); 7.5290 (1.9);
7.5095 (2.1); 7.5036 (2.2); 7.4948 (0.6); 7.4724 (1.4); 7.4677 (1.3); 7.4497 (1.4); 7.4449 (1.8); 7.4363 (2.2); 7.4312
(2.4); 7.4222 (1.5); 7.4171 (1.4); 7.4098 (1.1); 7.4050 (0.8); 7.3968 (1.8); 7.3922 (1.7); 7.3772 (0.5); 7.3589 (0.6);
7.3456 (3.0); 7.3251 (2.6); 7.3139 (1.5); 7.2990 (9.6); 7.2942 (1.8); 7.2837 (0.4); 7.2615 (1.5); 7.2560 (1.2); 7.2382
(1.3); 7.2361 (1.3); 7.2332 (1.3); 7.2305 (1.1); 7.2134 (0.8); 7.2077 (0.7); 7.1587 (2.0); 7.1427 (2.0); 7.1332 (1.8);
7.1172 (1.8); 5.4859 (1.8); 5.3376 (0.9); 2.5206 (16.0); 1.9237 (0.6); 1.9127 (0.7); 1.8970 (1.3); 1.8812 (0.8); 1.8701
(0.7); 1.8542 (0.3); 1.6233 (12.6); 1.2321 (0.4); 1.2277 (0.4); 1.2225 (0.4); 1.2119 (0.8); 1.2068 (0.5); 1.2011 (0.6);
1.1962 (0.8); 1.1913 (0.6); 1.1854 (0.6); 1.1800 (0.6); 1.1755 (0.5); 1.1643 (0.4); 1.0855 (0.4); 1.0747 (0.5); 1.0695
(0.5); 1.0646 (0.7); 1.0586 (0.6); 1.0541 (0.9); 1.0488 (0.6); 1.0438 (0.4); 1.0380 (0.8); 1.0337 (0.5); 1.0278 (0.4);
1.0227 (0.5); 1.0176 (0.4); 1.0067 (0.4); 0.9141 (0.4); 0.9028 (0.4); 0.8927 (0.4); 0.8835 (0.8); 0.8726 (0.6); 0.8628
(0.6); 0.8562 (0.8); 0.8454 (0.5); 0.8355 (0.4); 0.8244 (0.3); 0.7302 (0.4); 0.7195 (0.4); 0.7089 (0.5); 0.7032 (0.6);
0.6992 (0.8); 0.6929 (0.6); 0.6894 (0.5); 0.6823 (0.6); 0.6789 (0.5); 0.6723 (0.7); 0.6628 (0.4); 0.0484 (0.4); 0.0375
(11.7); 0.0267 (0.5)
I.060: 1H-NMR(300.2 MHz, CDCl3):
δ = 8.6741 (1.5); 8.6682 (1.6); 8.6581 (1.6); 8.6520 (1.6); 8.1413 (0.9); 7.7981 (2.6); 7.7940 (2.7); 7.5814 (1.5);
7.5754 (1.6); 7.5558 (1.8); 7.5499 (1.8); 7.4733 (0.5); 7.4679 (0.6); 7.4457 (1.3); 7.4412 (1.1); 7.4225 (1.1); 7.4170
(1.2); 7.3937 (0.5); 7.3891 (0.5); 7.3778 (1.8); 7.3686 (3.4); 7.3589 (1.5); 7.3484 (1.3); 7.3438 (1.5); 7.3387 (2.1);
7.3167 (1.2); 7.2991 (23.2); 7.2868 (0.8); 7.2731 (3.0); 7.2672 (2.0); 7.2581 (1.7); 7.2487 (1.5); 7.2325 (1.5); 7.2078
(1.3); 7.2037 (1.3); 7.1834 (1.5); 7.1797 (1.6); 7.1589 (0.7); 7.1546 (0.7); 5.3377 (0.4); 5.1889 (1.8); 2.7280 (1.0);
2.7199 (1.2); 2.7108 (0.5); 2.7012 (2.0); 2.6952 (2.0); 2.6866 (0.6); 2.6764 (1.2); 2.6686 (1.2); 2.4772 (16.0); 2.4523
(0.7); 2.0834 (1.1); 1.8380 (1.7); 1.7600 (0.7); 1.7395 (0.6); 1.7257 (0.5); 1.7143 (0.9); 1.7014 (0.9); 1.6889 (1.1);
1.6768 (1.2); 1.6628 (1.2); 1.6523 (1.1); 1.6382 (0.8); 1.6257 (0.7); 1.3209 (0.4); 1.2971 (1.0); 1.2923 (0.8); 1.2733
(0.4); 0.8524 (4.4); 0.8279 (9.1); 0.8033 (3.9); 0.0481 (0.9); 0.0373 (26.6); 0.0263 (0.9)
I.061: 1H-NMR(300.2 MHz, CDCl3):
δ = 8.6358 (0.6); 8.5626 (0.4); 8.5461 (0.4); 7.8102 (0.4); 7.3721 (0.6); 7.3415 (0.4); 7.2986 (6.9); 7.1934 (0.4);
7.1771 (0.6); 2.4813 (2.4); 1.6041 (16.0); 0.8364 (0.7); 0.8119 (1.3); 0.7875 (0.6); 0.0375 (7.8)
I.062: 1H-NMR(300.2 MHz, CDCl3):
δ = 8.6029 (2.8); 8.5860 (2.9); 8.4920 (4.1); 7.8073 (2.6); 7.8031 (2.6); 7.4812 (0.5); 7.4757 (0.6); 7.4530 (1.3);
7.4300 (1.1); 7.4245 (1.2); 7.4125 (0.6); 7.3950 (0.6); 7.3902 (0.5); 7.3787 (1.6); 7.3646 (3.3); 7.3608 (3.3); 7.3482
(1.3); 7.3376 (2.5); 7.3158 (1.7); 7.3074 (3.1); 7.2990 (41.0); 7.2768 (2.2); 7.2713 (2.0); 7.2110 (1.3); 7.2069 (1.3);
7.1862 (1.6); 7.1827 (1.7); 7.1618 (0.7); 7.1579 (0.7); 5.2008 (1.8); 2.5287 (2.1); 2.5030 (4.3); 2.4826 (16.0); 1.7230
(0.9); 1.6877 (1.0); 1.6416 (0.8); 1.6170 (0.7); 1.5918 (0.6); 1.5722 (1.0); 1.5476 (1.6); 1.5230 (1.7); 1.5018 (1.4);
1.4768 (0.8); 1.4570 (0.4); 1.2932 (0.7); 0.8260 (4.3); 0.8017 (8.7); 0.7771 (3.8); 0.1080 (0.9); 0.0488 (2.2); 0.0379
(53.7); 0.0271 (2.1)
I.064: 1H-NMR(400.2 MHz, d6-DMSO):
δ = 8.8271 (2.4); 8.8247 (2.7); 8.8224 (2.7); 8.7436 (4.1); 8.7386 (4.2); 8.4235 (1.8); 8.4188 (2.9); 8.4148 (1.6);
7.9559 (2.2); 7.8276 (3.4); 7.6055 (0.6); 7.6018 (0.6); 7.5916 (0.7); 7.5877 (0.8); 7.5824 (1.0); 7.5787 (1.1); 7.5685
(1.0); 7.5652 (1.0); 7.5316 (0.9); 7.5237 (1.0); 7.5150 (1.2); 7.5110 (1.8); 7.5058 (1.1); 7.4947 (2.1); 7.4907 (1.3);
7.4798 (1.1); 7.4743 (1.5); 7.4567 (0.5); 7.3588 (3.3); 7.1541 (1.2); 7.1327 (2.0); 7.1113 (1.0); 7.1095 (1.1); 7.0744
(2.3); 7.0542 (2.1); 3.3435 (36.9); 2.8939 (16.0); 2.7354 (13.5); 2.7343 (13.3); 2.5560 (16.5); 2.5298 (1.1); 2.5244
(1.1); 2.5157 (11.8); 2.5113 (23.5); 2.5068 (30.3); 2.5022 (21.8); 2.4977 (10.4); −0.0002 (1.7)
I.065: 1H-NMR(400.2 MHz, d6-DMSO):
δ = 8.1847 (3.2); 8.1790 (3.2); 7.9562 (1.6); 7.7585 (1.9); 7.7524 (1.8); 7.7371 (2.0); 7.7310 (1.9); 7.6021 (3.7);
7.4623 (0.8); 7.4586 (0.9); 7.4505 (0.7); 7.4423 (2.0); 7.4381 (2.1); 7.4265 (4.8); 7.4104 (4.5); 7.3988 (1.3); 7.3824
(1.1); 7.3588 (0.4); 7.3330 (1.7); 7.3163 (4.8); 7.2976 (3.1); 6.9640 (3.8); 6.6080 (3.1); 6.5866 (3.1); 4.1756 (1.6);
4.1581 (5.0); 4.1405 (5.0); 4.1229 (1.6); 3.3454 (33.7); 2.8933 (9.4); 2.7351 (8.6); 2.5971 (16.0); 2.5112 (21.3);
2.5070 (26.4); 2.5027 (19.6); 1.1781 (5.1); 1.1605 (10.5); 1.1430 (5.0); −0.0002 (0.9)
I.066: 1H-NMR(400.2 MHz, d6-DMSO):
δ = 7.9558 (2.0); 7.9367 (3.0); 7.9320 (3.3); 7.9244 (3.2); 7.9196 (3.2); 7.6221 (3.0); 7.6173 (3.1); 7.6038 (3.4);
7.5991 (3.1); 7.4577 (0.5); 7.4458 (1.2); 7.4419 (1.5); 7.4344 (1.9); 7.4218 (3.7); 7.4154 (4.2); 7.4084 (3.6); 7.4037
(3.3); 7.3987 (5.8); 7.3767 (0.4); 7.3580 (2.4); 7.3542 (2.4); 7.3388 (3.8); 7.3345 (4.0); 7.3321 (4.0); 7.3294 (4.0);
7.3111 (2.6); 7.3094 (2.6); 7.2949 (5.2); 7.2570 (2.1); 7.2541 (2.0); 7.2384 (3.0); 7.2358 (2.9); 7.2199 (1.3); 7.2169
(1.2); 7.1730 (5.2); 6.8565 (3.1); 6.8441 (3.1); 6.8382 (3.1); 6.8258 (2.9); 4.2453 (1.8); 4.2277 (5.8); 4.2101 (5.9);
4.1925 (1.9); 3.3430 (45.2); 2.8927 (13.2); 2.7341 (11.5); 2.5268 (25.4); 2.5150 (14.8); 2.5107 (28.8); 2.5062 (37.3);
2.5017 (27.4); 2.4975 (13.7); 1.2125 (7.7); 1.1950 (16.0); 1.1773 (7.4); −0.0002 (2.0)
I.068: 1H-NMR(300.2 MHz, CDCl3):
δ = 19.1321 (0.4); 9.6695 (0.4); 8.7245 (0.6); 8.7010 (0.7); 8.6605 (0.6); 8.6499 (0.7); 8.6441 (0.8); 8.6231 (0.9);
8.5853 (0.7); 8.5150 (0.8); 8.4717 (0.5); 7.8788 (0.7); 7.8449 (0.7); 7.3736 (4.8); 7.3542 (4.9); 7.3219 (5.4); 7.2985
(6.5); 7.2705 (9.7); 7.0624 (0.6); 6.9772 (1.0); 6.9546 (0.8); 6.9188 (0.7); 6.8885 (0.7); 6.8448 (0.5); 6.7993 (0.4);
5.1995 (0.8); 5.1718 (0.6); 5.1350 (0.4); 2.7041 (0.5); 2.6533 (0.8); 2.5806 (2.0); 2.5678 (2.1); 2.5358 (2.3); 2.5030
(2.2); 2.4755 (2.5); 1.8409 (0.5); 1.6064 (16.0); 1.5771 (16.0); 1.4117 (0.5); 1.3784 (0.7); 1.2920 (1.1); 1.2011 (1.8);
1.1930 (1.7); 1.1739 (2.0); 1.1470 (1.8); 0.0587 (5.7); 0.0370 (7.7); 0.0090 (11.5)
I.069: 1H-NMR(300.2 MHz, CDCl3):
δ = 8.7283 (0.6); 8.7225 (0.6); 8.7123 (0.6); 8.7064 (0.6); 7.8744 (1.1); 7.6291 (0.6); 7.6232 (0.6); 7.6036 (0.7);
7.5977 (0.7); 7.4264 (0.4); 7.4219 (0.5); 7.4090 (0.4); 7.4024 (0.8); 7.3794 (0.5); 7.3703 (1.1); 7.3488 (1.0); 7.3433
(0.5); 7.3367 (0.7); 7.3207 (0.9); 7.3108 (0.6); 7.2989 (6.3); 7.0232 (0.8); 6.9957 (0.7); 6.8996 (0.4); 6.8972 (0.4);
6.8702 (0.7); 6.8430 (0.4); 5.3373 (1.1); 5.1712 (0.8); 2.7655 (0.7); 2.7593 (0.7); 2.7401 (0.7); 2.7344 (0.7); 2.4972
(6.4); 1.6242 (16.0); 1.2917 (0.4); 1.2658 (1.8); 1.2407 (3.5); 1.2155 (1.6); 0.0367 (6.5)
I.070: 1H-NMR(400.2 MHz, d6-DMSO):
δ = 8.2746 (2.1); 7.7945 (2.1); 7.7137 (0.4); 7.7100 (0.4); 7.7000 (0.5); 7.6959 (0.5); 7.6908 (0.6); 7.6869 (0.6);
7.6769 (0.6); 7.6736 (0.6); 7.6002 (0.5); 7.5820 (0.6); 7.5746 (0.6); 7.5567 (0.6); 7.5516 (0.4); 7.5331 (0.3); 7.3797
(0.5); 7.3591 (1.0); 7.3421 (1.0); 7.3216 (0.5); 7.1321 (2.2); 6.9276 (0.8); 6.9062 (1.4); 6.8849 (0.7); 6.6936 (1.5);
6.6732 (1.5); 3.3457 (19.4); 2.5111 (12.2); 2.5067 (15.3); 2.5022 (11.0); 2.4981 (5.4); 2.4767 (9.9); 2.1007
(16.0); −0.0002 (0.9)
I.071: 1H-NMR(400.2 MHz, d6-DMSO):
δ = 8.4992 (2.4); 8.4948 (2.4); 7.9552 (1.4); 7.7983 (0.3); 7.7880 (0.3); 7.7378 (1.5); 7.7320 (1.4); 7.7169 (1.6);
7.7112 (1.6); 7.6264 (2.9); 7.4801 (1.0); 7.4653 (2.1); 7.4616 (2.2); 7.4542 (2.0); 7.4510 (2.0); 7.4426 (3.1); 7.4348
(3.4); 7.4183 (1.1); 7.4099 (1.1); 7.3931 (0.9); 7.3698 (0.4); 7.3522 (1.3); 7.3360 (1.6); 7.3159 (2.4); 7.2964 (1.6);
7.1563 (2.2); 7.1356 (2.1); 7.0118 (2.9); 3.6140 (1.1); 3.3459 (33.4); 2.8925 (9.0); 2.7342 (7.9); 2.6018 (12.4);
2.5104 (19.1); 2.5061 (23.6); 2.5017 (17.2); 2.3963 (16.0); −0.0002 (0.7)
I.072: 1H-NMR(400.2 MHz, d6-DMSO):
δ = 8.1620 (1.6); 8.1577 (1.7); 8.1500 (1.7); 8.1456 (1.6); 7.9533 (0.5); 7.7130 (0.3); 7.6966 (0.3); 7.6931 (0.3);
7.6138 (0.6); 7.5411 (1.6); 7.5368 (1.7); 7.5223 (1.9); 7.5179 (1.7); 7.4781 (0.7); 7.4741 (0.8); 7.4537 (3.1); 7.4397
(1.3); 7.4360 (1.7); 7.4206 (1.0); 7.4112 (1.9); 7.3952 (2.4); 7.3870 (0.5); 7.3715 (0.8); 7.3647 (1.4); 7.3606 (1.3);
7.3455 (1.7); 7.3417 (1.5); 7.2925 (1.7); 7.2747 (1.3); 7.2726 (1.3); 7.2508 (3.4); 7.2481 (3.4); 7.2321 (1.6); 7.2294
(1.5); 7.2134 (0.7); 7.2105 (0.6); 7.1573 (0.3); 6.9344 (1.6); 6.9223 (1.6); 6.9156 (1.6); 6.9035 (1.5); 3.3332 (67.2);
2.8907 (3.7); 2.7309 (3.1); 2.6759 (0.4); 2.6671 (2.9); 2.5283 (13.5); 2.5205 (3.1); 2.5117 (31.2); 2.5073 (62.8);
2.5027 (81.6); 2.4982 (58.8); 2.4937 (28.3); 2.3397 (16.0); 2.3298 (0.8); 2.3252 (0.5); 1.2342 (0.6); −0.0002 (0.6)
I.073: 1H-NMR(400.2 MHz, d6-DMSO):
δ = 8.0687 (2.2); 8.0629 (2.2); 7.9567 (0.6); 7.7830 (2.4); 7.7457 (0.5); 7.7417 (0.6); 7.7316 (0.6); 7.7277 (0.7);
7.7228 (0.8); 7.7188 (0.8); 7.7088 (0.7); 7.7055 (0.7); 7.6304 (1.8); 7.6246 (1.4); 7.6088 (1.9); 7.6036 (1.9); 7.5869
(0.8); 7.5821 (0.6); 7.5635 (0.4); 7.4426 (0.5); 7.4276 (0.7); 7.4227 (1.1); 7.4079 (1.1); 7.4028 (0.9); 7.3878 (0.7);
7.2771 (0.9); 7.2555 (1.5); 7.2341 (0.7); 7.0263 (1.7); 7.0074 (1.5); 6.8399 (2.2); 6.8390 (2.2); 6.8187 (2.2); 6.8176
(2.1); 4.0512 (5.0); 3.8382 (16.0); 3.3443 (35.1); 2.8961 (4.2); 2.7372 (3.6); 2.5309 (0.4); 2.5175 (7.7); 2.5132
(15.3); 2.5087 (19.9); 2.5042 (14.8); 2.4999 (7.7); 2.4097 (12.5); −0.0002 (0.4)
I.074: 1H-NMR(400.2 MHz, d6-DMSO):
δ = 8.1352 (1.3); 8.1306 (1.4); 8.1226 (1.6); 8.1138 (3.1); 7.9573 (0.7); 7.8041 (0.7); 7.7998 (0.7); 7.7902 (0.7);
7.7854 (0.8); 7.7813 (1.0); 7.7771 (0.9); 7.7675 (0.9); 7.7635 (0.8); 7.6785 (2.3); 7.6583 (1.2); 7.6489 (0.8); 7.6381
(0.9); 7.6326 (1.0); 7.6216 (1.4); 7.6167 (1.9); 7.6037 (1.7); 7.5988 (1.4); 7.5894 (1.1); 7.5831 (0.7); 7.5653 (0.6);
7.4758 (0.3); 7.4472 (0.5); 7.4320 (0.8); 7.4277 (1.0); 7.4127 (1.2); 7.4076 (0.9); 7.4028 (0.6); 7.3924 (0.8); 7.3838
(1.1); 7.3664 (1.4); 7.3470 (0.9); 7.3259 (0.5); 7.2386 (0.8); 7.2159 (1.3); 7.1944 (0.7); 7.0938 (0.9); 7.0839 (1.8);
7.0755 (3.2); 7.0628 (2.3); 7.0596 (2.4); 7.0523 (2.4); 7.0418 (2.2); 7.0298 (1.4); 7.0173 (1.4); 7.0118 (1.2); 6.9992
(1.1); 6.7077 (1.0); 6.6867 (1.0); 4.2157 (7.1); 4.0472 (0.8); 4.0061 (1.7); 3.9427 (1.6); 3.9016 (0.8); 3.6175 (5.8);
3.5570 (12.2); 3.3444 (62.0); 2.8966 (4.3); 2.7385 (3.9); 2.6096 (16.0); 2.5135 (29.1); 2.5091 (36.6); 2.5048 (26.6);
2.3778 (10.7); 1.6436 (0.5); 1.6112 (0.6); 1.5562 (0.6); 1.2352 (0.7); 1.0554 (1.0); −0.0002 (0.5)
I.075: 1H-NMR(400.2 MHz, d6-DMSO):
δ = 8.1342 (1.6); 8.1295 (1.8); 8.1216 (2.0); 8.1166 (3.1); 7.9565 (0.4); 7.8037 (0.4); 7.7993 (0.5); 7.7897 (0.5);
7.7850 (0.7); 7.7810 (0.8); 7.7771 (0.8); 7.7670 (0.7); 7.7632 (0.7); 7.6751 (3.1); 7.6570 (1.5); 7.6444 (1.0); 7.6377
(0.8); 7.6303 (0.8); 7.6207 (1.7); 7.6159 (2.1); 7.6028 (2.2); 7.5980 (1.9); 7.5886 (1.3); 7.5812 (1.0); 7.5633 (0.9);
7.5397 (0.4); 7.4478 (0.6); 7.4325 (0.8); 7.4278 (1.3); 7.4129 (1.3); 7.4080 (1.2); 7.4033 (0.6); 7.3930 (0.9); 7.3841
(0.8); 7.3666 (1.0); 7.3471 (0.6); 7.2379 (1.0); 7.2157 (1.7); 7.1940 (0.9); 7.0857 (2.1); 7.0726 (2.3); 7.0676 (2.6);
7.0590 (1.8); 7.0505 (1.7); 7.0409 (1.6); 7.0304 (1.9); 7.0179 (1.8); 7.0124 (1.8); 6.9998 (1.6); 6.7098 (0.3); 4.3062
(0.4); 4.2151 (4.9); 4.0472 (1.0); 4.0060 (2.2); 3.9427 (2.2); 3.9015 (1.0); 3.6180 (1.8); 3.5545 (16.0); 3.3681
(148.4); 2.8969 (2.3); 2.7384 (2.1); 2.6746 (1.0); 2.6087 (11.0); 2.5147 (27.7); 2.5105 (36.3); 2.5061 (27.9); 2.3769
(13.8); 1.2338 (0.4)
I.076: 1H-NMR(400.2 MHz, d6-DMSO):
δ = 7.9548 (0.4); 7.9480 (1.6); 7.9433 (1.8); 7.9356 (1.8); 7.9308 (1.8); 7.6311 (1.5); 7.6264 (1.6); 7.6129 (1.7);
7.6081 (1.7); 7.5026 (2.7); 7.4924 (0.8); 7.4832 (2.6); 7.4676 (2.2); 7.4580 (1.0); 7.4420 (0.9); 7.4350 (0.8); 7.4183
(1.1); 7.4146 (1.3); 7.3980 (1.3); 7.3943 (1.0); 7.3776 (0.7); 7.3175 (2.9); 7.0209 (3.4); 7.0002 (4.0); 6.9787 (1.0);
6.8661 (1.7); 6.8537 (1.7); 6.8479 (1.8); 6.8354 (1.6); 3.7590 (16.0); 3.3404 (25.1); 2.8939 (2.5); 2.7357 (2.2);
2.6876 (1.0); 2.5120 (22.4); 2.5067 (21.9); 2.5022 (16.1); 2.4979 (8.5); 1.2364 (0.8); −0.0002 (0.5)
I.077: 1H-NMR(400.2 MHz, d6-DMSO):
δ = 8.1565 (2.1); 8.1543 (1.9); 7.9548 (1.2); 7.7092 (1.1); 7.7047 (1.2); 7.6880 (1.2); 7.6834 (1.2); 7.5784 (2.7);
7.5680 (0.7); 7.5641 (0.6); 7.5536 (0.6); 7.5500 (0.7); 7.5445 (0.9); 7.5409 (0.9); 7.5304 (0.8); 7.5275 (0.8); 7.4855
(0.6); 7.4673 (0.7); 7.4599 (0.8); 7.4417 (0.8); 7.4370 (1.1); 7.4167 (1.5); 7.4002 (1.3); 7.3963 (0.9); 7.3797 (0.7);
7.3083 (2.7); 7.0980 (0.9); 7.0765 (1.5); 7.0552 (0.8); 7.0536 (0.8); 7.0271 (1.8); 7.0070 (1.6); 6.6918 (2.3); 6.6906
(2.4); 6.6705 (2.2); 6.6692 (2.2); 3.7149 (16.0); 3.3365 (24.3); 2.8946 (8.2); 2.7360 (7.0); 2.6878 (1.0); 2.5510
(12.9); 2.5292 (0.6); 2.5156 (8.8); 2.5113 (17.7); 2.5068 (23.2); 2.5023 (17.4); 2.4981 (9.0); 1.2372 (0.8); −0.0002
(0.8)
I.078: 1H-NMR(400.2 MHz, d6-DMSO):
δ = 8.5715 (10.9); 7.9560 (1.0); 7.7866 (3.1); 7.7484 (0.7); 7.7338 (0.9); 7.7290 (1.1); 7.7259 (1.0); 7.7150 (1.0);
7.6372 (0.7); 7.6187 (0.8); 7.6122 (1.0); 7.5937 (0.9); 7.5704 (0.5); 7.4947 (0.6); 7.4747 (1.3); 7.4597 (1.3); 7.4550
(1.1); 7.4397 (0.8); 7.3247 (1.1); 7.3019 (1.8); 7.2805 (0.9); 7.0639 (2.0); 7.0447 (1.9); 4.1063 (6.3); 3.8992 (16.0);
3.3502 (68.5); 2.8968 (5.5); 2.7380 (5.2); 2.5131 (19.4); 2.5091 (25.0); 2.5050 (20.0); 2.4433 (14.1)
I.079: 1H-NMR(400.2 MHz, d6-DMSO):
δ = 7.7524 (2.0); 7.7494 (2.1); 7.7400 (2.2); 7.7369 (2.1); 7.5209 (0.5); 7.5043 (0.7); 7.4970 (2.0); 7.4867 (2.1);
7.4828 (2.5); 7.4728 (1.4); 7.4644 (2.9); 7.4441 (4.8); 7.4244 (0.5); 7.3884 (1.3); 7.3844 (1.8); 7.3677 (2.0); 7.3646
(1.9); 7.2965 (3.3); 7.2944 (3.4); 7.2673 (3.1); 7.2562 (6.6); 7.2375 (4.7); 7.2206 (1.0); 7.2182 (0.8); 3.7574 (10.7);
3.7516 (10.5); 3.6150 (1.4); 3.3442 (31.4); 2.8936 (0.8); 2.7346 (0.7); 2.5734 (16.0); 2.5289 (0.5); 2.5153 (10.2);
2.5111 (20.0); 2.5067 (25.7); 2.5022 (18.9); 2.4981 (9.5); −0.0002 (1.6)
I.080: 1H-NMR(400.2 MHz, d6-DMSO):
δ = 8.1267 (2.8); 7.7466 (2.6); 7.7384 (1.7); 7.7198 (2.0); 7.7176 (1.9); 7.6991 (1.7); 7.6292 (0.5); 7.6253 (0.5);
7.6154 (0.5); 7.6114 (0.6); 7.6062 (0.8); 7.6023 (0.8); 7.5923 (0.8); 7.5889 (0.7); 7.5415 (0.6); 7.5234 (0.7); 7.5158
(0.8); 7.4978 (0.7); 7.4930 (0.5); 7.4746 (0.4); 7.4194 (0.6); 7.4030 (0.7); 7.3989 (1.2); 7.3826 (1.3); 7.3784 (0.8);
7.3620 (0.6); 7.1857 (1.1); 7.1799 (1.1); 7.1674 (1.0); 7.1616 (1.1); 7.0551 (1.8); 7.0346 (1.6); 6.9884 (0.9); 6.9867
(0.9); 6.9639 (1.2); 6.9427 (0.8); 6.7175 (2.2); 6.7164 (2.3); 6.6969 (2.2); 6.6956 (2.2); 3.8416 (16.0); 3.3441 (28.2);
2.8941 (1.1); 2.7358 (1.0); 2.7347 (1.0); 2.5983 (12.4); 2.5297 (0.4); 2.5161 (8.0); 2.5118 (15.8); 2.5072 (20.4);
2.5027 (14.8); 2.4983 (7.3); −0.0002 (1.4)
I.081: 1H-NMR(400.2 MHz, d6-DMSO):
δ = 8.1999 (3.6); 8.0804 (1.7); 8.0687 (1.7); 7.9547 (0.6); 7.5356 (0.4); 7.5119 (2.5); 7.4955 (3.6); 7.4855 (1.5);
7.4692 (1.0); 7.4574 (0.7); 7.4452 (0.6); 7.4369 (1.2); 7.4203 (1.2); 7.4000 (0.6); 7.3634 (2.4); 7.2525 (1.4); 7.2408
(1.4); 7.0272 (1.0); 7.0106 (2.1); 7.0060 (2.0); 6.9911 (1.6); 6.9848 (1.1); 3.7904 (16.0); 3.7050 (0.4); 3.6562 (0.8);
3.6138 (0.4); 3.3466 (55.9); 2.8923 (4.7); 2.7341 (3.8); 2.7331 (3.8); 2.5473 (0.4); 2.5200 (16.1); 2.5144 (13.2);
2.5099 (24.4); 2.5054 (31.1); 2.5008 (22.2); 2.4963 (10.6); −0.0002 (0.5)
I.082: 1H-NMR(400.2 MHz, d6-DMSO):
δ = 8.2333 (4.8); 8.2283 (3.1); 8.2137 (2.6); 7.9550 (1.6); 7.5339 (0.4); 7.5180 (0.6); 7.5102 (1.9); 7.5033 (1.4);
7.4952 (1.6); 7.4797 (3.9); 7.4625 (1.0); 7.4471 (0.8); 7.4392 (0.4); 7.4305 (0.9); 7.4268 (1.4); 7.4102 (1.5); 7.4065
(1.0); 7.3898 (0.8); 7.3445 (3.0); 7.0292 (1.1); 7.0098 (3.7); 6.9896 (2.4); 6.8862 (2.8); 6.8716 (2.7); 3.7241 (16.0);
3.3471 (8.2); 2.8926 (10.8); 2.7342 (9.4); 2.7333 (9.1); 2.5550 (0.4); 2.5278 (0.7); 2.5143 (11.6); 2.5100 (22.7);
2.5054 (30.2); 2.5007 (32.8); −0.0002 (1.6)
I.083: 1H-NMR(400.2 MHz, d6-DMSO):
δ = 7.9957 (2.2); 7.9837 (2.2); 7.9825 (2.3); 7.9547 (1.6); 7.6062 (2.6); 7.5802 (0.5); 7.5770 (0.5); 7.5667 (0.5);
7.5628 (0.6); 7.5574 (0.8); 7.5538 (0.8); 7.5433 (0.8); 7.5404 (0.8); 7.5052 (0.6); 7.4871 (0.7); 7.4796 (0.8); 7.4667
(0.9); 7.4564 (0.6); 7.4505 (0.8); 7.4464 (1.3); 7.4383 (0.5); 7.4302 (1.3); 7.4261 (0.8); 7.4096 (0.7); 7.3884 (2.5);
7.0881 (0.9); 7.0663 (1.4); 7.0447 (0.8); 7.0429 (0.8); 7.0179 (1.8); 6.9977 (1.6); 6.9605 (1.6); 6.9473 (1.6); 6.8278
(2.8); 3.7044 (16.0); 3.3456 (56.8); 2.8920 (11.2); 2.7337 (9.3); 2.7326 (9.6); 2.5470 (12.7); 2.5277 (0.6); 2.5142
(11.4); 2.5098 (22.7); 2.5053 (29.3); 2.5007 (21.2); 2.4963 (10.4)
I.084: 1H-NMR(400.2 MHz, d6-DMSO):
δ = 7.9549 (1.7); 7.9011 (3.0); 7.8968 (3.2); 7.8953 (3.1); 7.6790 (3.3); 7.6744 (3.2); 7.6732 (3.1); 7.6291 (4.0);
7.5083 (0.6); 7.4931 (1.7); 7.4887 (1.8); 7.4841 (2.2); 7.4725 (4.3); 7.4552 (2.3); 7.4506 (2.3); 7.4312 (1.2); 7.4258
(0.5); 7.4081 (0.5); 7.3484 (1.7); 7.3447 (1.8); 7.3295 (2.7); 7.3256 (2.6); 7.2848 (2.9); 7.2704 (2.3); 7.2674 (3.4);
7.2521 (2.6); 7.2495 (2.3); 7.2335 (1.1); 7.2307 (1.0); 7.1511 (3.9); 3.3449 (55.8); 2.8924 (11.5); 2.7333 (9.9);
2.5492 (19.6); 2.5282 (0.7); 2.5146 (12.4); 2.5103 (24.9); 2.5058 (32.5); 2.5012 (23.8); 2.4968 (11.9); 2.0769
(16.0); −0.0002 (1.8)
I.085: 1H-NMR(400.2 MHz, d6-DMSO):
δ = 7.9545 (1.9); 7.8268 (0.5); 7.8182 (0.4); 7.8108 (0.4); 7.7987 (0.5); 7.7942 (0.5); 7.7867 (0.6); 7.6849 (3.4);
7.6646 (3.6); 7.6341 (1.0); 7.6249 (0.5); 7.6147 (0.5); 7.5940 (0.6); 7.5465 (0.8); 7.5377 (1.1); 7.5337 (1.0); 7.5247
(1.8); 7.5106 (1.4); 7.5010 (3.6); 7.4875 (1.2); 7.4804 (0.9); 7.4759 (1.5); 7.4692 (1.9); 7.4627 (2.2); 7.4566 (2.0);
7.4527 (2.0); 7.4438 (1.2); 7.4382 (1.8); 7.4337 (1.5); 7.4230 (1.4); 7.4187 (1.3); 7.4102 (0.7); 7.4061 (0.6); 7.3889
(0.7); 7.3587 (0.7); 7.3482 (0.9); 7.3412 (1.1); 7.3155 (1.1); 7.3114 (1.3); 7.2966 (2.6); 7.2924 (2.3); 7.2702 (1.6);
7.2678 (1.8); 7.2519 (1.8); 7.2494 (2.1); 7.2339 (2.9); 7.2151 (2.1); 7.1966 (3.4); 7.1691 (2.9); 7.1489 (2.7); 7.0496
(0.5); 7.0177 (0.5); 6.9295 (0.7); 6.8888 (0.6); 6.5849 (0.7); 6.5529 (0.6); 3.3404 (78.3); 2.8917 (13.3); 2.7331
(11.1); 2.7322 (11.3); 2.6736 (0.3); 2.5339 (17.1); 2.5135 (20.8); 2.5092 (40.7); 2.5047 (52.3); 2.5001 (38.0); 2.4957
(18.7); 2.3282 (16.0); 1.2324 (0.3)
I.086: 1H-NMR(400.2 MHz, d6-DMSO):
δ = 8.1905 (6.7); 7.9545 (1.7); 7.8268 (1.0); 7.8180 (1.0); 7.8112 (0.8); 7.7986 (1.2); 7.7941 (1.0); 7.7867 (1.2);
7.6341 (1.7); 7.6250 (0.9); 7.6146 (0.9); 7.5940 (1.0); 7.5602 (0.7); 7.5386 (2.0); 7.5240 (2.1); 7.4918 (2.4); 7.4814
(5.1); 7.4743 (3.7); 7.4672 (4.7); 7.4630 (3.0); 7.4546 (2.0); 7.4496 (2.4); 7.4230 (2.4); 7.4180 (2.0); 7.4099 (1.2);
7.4058 (1.0); 7.3889 (1.5); 7.3812 (0.4); 7.3729 (0.6); 7.3672 (0.4); 7.3587 (1.2); 7.3483 (1.8); 7.3410 (2.0); 7.3282
(0.4); 7.3250 (0.3); 7.3068 (0.9); 7.3019 (1.2); 7.2879 (3.3); 7.2828 (3.4); 7.2768 (7.5); 7.2577 (2.1); 7.2362 (2.8);
7.2153 (5.6); 7.0493 (0.8); 7.0174 (0.9); 6.9295 (1.2); 6.8889 (1.1); 6.5848 (1.3); 6.5528 (1.1); 3.3377 (59.2); 2.8914
(11.7); 2.7321 (9.9); 2.6731 (0.4); 2.5247 (19.4); 2.5132 (24.7); 2.5089 (48.4); 2.5044 (62.5); 2.4998 (45.5); 2.4954
(22.3); 2.3357 (0.4); 2.3276 (0.5); 2.1873 (16.0); 1.2320 (0.4); −0.0002 (0.5)
I.087: 1H-NMR(400.2 MHz, d6-DMSO):
δ = 8.2551 (2.9); 8.2496 (3.0); 7.9553 (1.9); 7.8765 (2.8); 7.8712 (2.7); 7.8274 (0.5); 7.8182 (0.5); 7.8115 (0.5);
7.7988 (0.6); 7.7947 (0.5); 7.7877 (0.7); 7.6802 (3.5); 7.6346 (0.9); 7.6255 (0.5); 7.6152 (0.5); 7.5947 (0.6); 7.5445
(0.5); 7.5397 (0.5); 7.5250 (0.7); 7.5024 (0.9); 7.4990 (1.0); 7.4801 (3.0); 7.4638 (2.8); 7.4553 (3.3); 7.4508 (3.4);
7.4410 (2.2); 7.4344 (3.3); 7.4184 (1.4); 7.4101 (1.6); 7.3890 (0.9); 7.3596 (0.7); 7.3484 (1.0); 7.3356 (1.8); 7.3161
(4.9); 7.2966 (2.9); 7.0493 (0.6); 7.0385 (3.6); 7.0179 (0.5); 6.9298 (0.6); 6.8892 (0.5); 6.5851 (0.6); 6.5532 (0.6);
3.3386 (34.3); 2.8921 (11.8); 2.7333 (10.8); 2.6730 (0.4); 2.5974 (16.0); 2.5096 (34.0); 2.5053 (42.9); 2.5011 (31.8);
2.3316 (0.4); 2.1818 (14.6); −0.0002 (0.4)
I.088: 1H-NMR(400.2 MHz, d6-DMSO):
δ = 8.6204 (8.3); 7.7339 (3.1); 7.6047 (0.5); 7.6014 (0.6); 7.5907 (0.6); 7.5875 (0.7); 7.5818 (0.9); 7.5786 (1.0);
7.5679 (0.8); 7.5652 (0.8); 7.5180 (0.7); 7.4998 (0.7); 7.4926 (0.9); 7.4749 (1.4); 7.4544 (1.5); 7.4380 (1.4); 7.4175
(0.7); 7.3864 (3.0); 7.1204 (1.0); 7.0985 (1.7); 7.0769 (0.9); 7.0075 (2.0); 6.9873 (1.8); 3.7977 (16.0); 3.3660 (79.3);
2.8965 (1.6); 2.7381 (1.5); 2.5733 (13.5); 2.5318 (0.5); 2.5182 (8.4); 2.5142 (16.5); 2.5097 (21.3); 2.5053 (15.9);
1.2370 (0.7)
I.089: 1H-NMR(400.2 MHz, d6-DMSO):
δ = 7.9870 (2.9); 7.9561 (2.3); 7.8687 (1.5); 7.8444 (1.5); 7.6886 (4.2); 7.5948 (0.8); 7.5911 (0.8); 7.5810 (0.9);
7.5771 (1.0); 7.5718 (1.3); 7.5681 (1.3); 7.5578 (1.2); 7.5546 (1.2); 7.5126 (1.0); 7.4945 (1.1); 7.4869 (1.3); 7.4700
(2.0); 7.4641 (0.9); 7.4535 (1.4); 7.4495 (2.2); 7.4332 (2.1); 7.4292 (1.4); 7.4126 (1.1); 7.3491 (4.1); 7.0985 (1.5);
7.0772 (2.4); 7.0559 (1.2); 7.0540 (1.3); 7.0321 (2.8); 7.0119 (2.6); 3.6158 (1.3); 3.3560 (96.9); 2.8943 (16.0);
2.7359 (13.7); 2.7349 (13.3); 2.5487 (20.6); 2.5302 (0.9); 2.5167 (13.8); 2.5123 (27.1); 2.5078 (34.8); 2.5032 (25.0);
2.4988 (12.0); 2.0689 (13.4); −0.0002 (0.3)
I.090: 1H-NMR(400.2 MHz, d6-DMSO):
δ = 8.4389 (10.6); 8.4379 (10.6); 8.4172 (16.0); 7.9552 (0.9); 7.8929 (0.5); 7.8902 (0.5); 7.8129 (0.6); 7.6482 (7.1);
7.5368 (2.0); 7.5327 (2.2); 7.5287 (1.3); 7.5170 (4.2); 7.5145 (4.6); 7.5038 (3.9); 7.4983 (3.9); 7.4941 (6.2); 7.4779
(2.2); 7.4695 (2.4); 7.4525 (2.1); 7.4467 (0.9); 7.4293 (0.9); 7.4053 (3.2); 7.4011 (3.6); 7.3851 (5.3); 7.3814 (4.2);
7.3509 (0.4); 7.3322 (0.6); 7.3298 (0.7); 7.3114 (0.6); 7.2934 (2.5); 7.2907 (3.5); 7.2753 (8.2); 7.2555 (8.1); 7.2487
(6.8); 7.2081 (0.6); 7.2055 (0.7); 7.1867 (0.6); 6.9791 (0.4); 3.6644 (0.5); 3.6144 (0.8); 3.3462 (130.2); 2.8932 (6.2);
2.7349 (5.2); 2.7338 (5.2); 2.6993 (3.0); 2.6799 (0.3); 2.6753 (0.4); 2.6707 (0.4); 2.5499 (34.2); 2.5288 (1.5); 2.5240
(2.2); 2.5153 (27.5); 2.5109 (55.0); 2.5063 (71.1); 2.5017 (51.1); 2.4972 (24.7); 2.3533 (0.6); 2.3377 (0.3); 2.3331
(0.4); 2.3285 (0.3); 1.2310 (0.3); −0.0002 (2.6)
I.091: 1H-NMR(400.2 MHz, d6-DMSO):
δ = 8.2124 (2.8); 8.2114 (2.8); 8.1997 (2.9); 8.1986 (2.9); 8.0682 (0.4); 7.7986 (3.3); 7.5950 (0.6); 7.5911 (0.6);
7.5811 (0.7); 7.5770 (0.8); 7.5662 (4.2); 7.5550 (1.0); 7.5186 (0.8); 7.5107 (0.9); 7.5022 (1.1); 7.4981 (1.8); 7.4927
(1.1); 7.4819 (2.1); 7.4777 (1.3); 7.4669 (1.1); 7.4614 (1.4); 7.4437 (0.5); 7.4068 (1.4); 7.4042 (2.1); 7.3915 (2.0);
7.3760 (3.2); 7.1269 (1.2); 7.1053 (1.8); 7.0835 (1.0); 7.0816 (1.1); 7.0691 (2.2); 7.0488 (2.0); 3.3445 (33.3); 2.8938
(1.5); 2.7355 (1.3); 2.7344 (1.3); 2.6880 (1.3); 2.5743 (16.0); 2.5296 (0.6); 2.5247 (0.8); 2.5161 (10.0); 2.5116
(20.0); 2.5071 (25.8); 2.5025 (18.6); 2.4980 (8.9); −0.0002 (2.0)
I.092: 1H-NMR(400.2 MHz, d6-DMSO):
δ = 8.4191 (2.2); 8.4167 (2.4); 8.4143 (2.2); 7.9564 (0.4); 7.8980 (1.4); 7.8934 (1.3); 7.8773 (1.5); 7.8727 (1.4);
7.7227 (3.4); 7.5861 (0.6); 7.5825 (0.6); 7.5720 (0.7); 7.5684 (0.8); 7.5630 (1.1); 7.5594 (1.1); 7.5489 (1.0); 7.5459
(1.0); 7.5114 (0.8); 7.4943 (1.4); 7.4858 (1.1); 7.4783 (1.2); 7.4742 (1.9); 7.4680 (1.2); 7.4623 (0.9); 7.4579 (1.8);
7.4539 (1.2); 7.4445 (0.6); 7.4374 (0.9); 7.4025 (3.0); 7.4012 (3.1); 7.3818 (2.8); 7.3805 (2.9); 7.3577 (3.2); 7.3556
(3.2); 7.1335 (1.1); 7.1121 (1.9); 7.0907 (1.0); 7.0888 (1.0); 7.0435 (2.2); 7.0233 (2.0); 3.3461 (35.6); 2.8938 (2.6);
2.7357 (2.2); 2.7346 (2.2); 2.6883 (0.9); 2.5604 (16.0); 2.5297 (0.6); 2.5249 (0.9); 2.5163 (10.0); 2.5119 (19.8);
2.5074 (25.6); 2.5028 (18.5); 2.4983 (8.9); −0.0002 (1.7)
I.093: 1H-NMR(400.2 MHz, d6-DMSO):
δ = 8.4984 (5.8); 8.3815 (3.6); 8.3691 (3.8); 8.0676 (0.7); 7.9558 (2.3); 7.6974 (3.7); 7.5961 (0.6); 7.5934 (0.6);
7.5820 (0.7); 7.5789 (0.8); 7.5703 (1.5); 7.5590 (1.3); 7.5564 (1.3); 7.5438 (1.2); 7.5262 (1.1); 7.5181 (1.4); 7.5151
(1.4); 7.4951 (5.1); 7.4833 (3.4); 7.4777 (2.7); 7.4740 (1.6); 7.4600 (3.9); 7.0672 (1.3); 7.0457 (2.3); 7.0228 (1.2);
6.9784 (2.7); 6.9582 (2.6); 3.6625 (0.7); 3.6145 (0.4); 3.3432 (40.6); 2.8932 (16.0); 2.7341 (13.9); 2.6873 (2.2);
2.5405 (18.6); 2.5292 (1.0); 2.5152 (12.8); 2.5109 (25.8); 2.5064 (33.6); 2.5018 (24.8); 2.4974 (12.4); −0.0002 (2.3)
I.094: 1H-NMR(400.2 MHz, d6-DMSO):
δ = 8.2115 (2.3); 8.2067 (2.6); 8.1996 (2.6); 8.1948 (2.5); 7.9554 (2.3); 7.9079 (2.4); 7.9030 (2.4); 7.8889 (2.7);
7.8840 (2.5); 7.6718 (3.8); 7.5997 (0.6); 7.5963 (0.6); 7.5858 (0.7); 7.5821 (0.8); 7.5765 (1.3); 7.5733 (1.4); 7.5627
(1.2); 7.5596 (1.2); 7.5401 (1.1); 7.5224 (1.1); 7.5146 (1.2); 7.4968 (1.1); 7.4892 (1.2); 7.4718 (4.9); 7.4686 (5.5);
7.4519 (2.0); 7.4480 (1.3); 7.4313 (1.0); 7.3226 (2.6); 7.3107 (2.5); 7.3037 (2.5); 7.2917 (2.4); 7.0590 (1.3); 7.0573
(1.4); 7.0361 (2.4); 7.0150 (1.2); 7.0130 (1.2); 6.9609 (2.6); 6.9406 (2.4); 3.3566 (99.8); 2.8935 (16.0); 2.7351
(13.3); 2.7343 (13.4); 2.6873 (0.9); 2.5303 (19.4); 2.5160 (12.7); 2.5116 (24.9); 2.5070 (31.9); 2.5024 (22.9); 2.4980
(11.1); −0.0002 (2.3)
I.095: 1H-NMR(400.2 MHz, d6-DMSO):
δ = 8.1178 (1.4); 8.1113 (1.7); 8.0975 (1.4); 8.0910 (1.6); 8.0383 (2.3); 8.0354 (2.5); 8.0320 (2.2); 8.0289 (1.9);
7.9549 (2.3); 7.7254 (3.3); 7.5249 (1.1); 7.5209 (1.1); 7.5165 (0.8); 7.5055 (3.2); 7.4892 (2.2); 7.4826 (1.5); 7.4719
(1.0); 7.4638 (1.2); 7.4480 (2.3); 7.4310 (1.9); 7.2880 (4.0); 7.2686 (3.5); 7.2530 (1.1); 7.2505 (0.8); 7.1984 (3.2);
3.3374 (29.8); 2.8921 (16.0); 2.7332 (13.6); 2.6693 (0.3); 2.5647 (16.0); 2.5269 (0.8); 2.5135 (16.5); 2.5092 (31.4);
2.5047 (39.6); 2.5002 (28.3); 2.4958 (13.7); 1.9105 (0.4); 1.2323 (0.3)
I.096: 1H-NMR(400.2 MHz, d6-DMSO):
δ = 7.9847 (2.5); 7.9721 (2.7); 7.8265 (0.4); 7.8180 (0.4); 7.8112 (0.4); 7.7986 (0.5); 7.7942 (0.4); 7.7865 (0.6);
7.6534 (3.3); 7.6341 (0.5); 7.5298 (1.2); 7.5268 (1.2); 7.5197 (0.5); 7.5111 (2.3); 7.5058 (4.0); 7.4890 (3.5); 7.4800
(1.7); 7.4633 (1.8); 7.4407 (0.4); 7.4231 (0.5); 7.4180 (0.6); 7.4068 (3.2); 7.3942 (3.0); 7.3882 (2.1); 7.3837 (1.8);
7.3677 (2.1); 7.3644 (2.0); 7.3482 (0.7); 7.3412 (0.5); 7.2782 (3.4); 7.2685 (3.1); 7.2518 (2.5); 7.2483 (2.4); 7.2352
(1.0); 3.3373 (52.7); 2.8916 (1.7); 2.7322 (1.6); 2.6728 (0.3); 2.5704 (16.0); 2.5267 (0.9); 2.5129 (17.6); 2.5089
(34.9); 2.5044 (45.4); 2.4999 (33.5); 2.4956 (16.8); 1.2322 (0.3)
I.097: 1H-NMR(400.2 MHz, d6-DMSO):
δ = 8.1908 (3.7); 8.1833 (3.8); 7.9545 (2.3); 7.9306 (1.6); 7.9231 (1.5); 7.9087 (1.7); 7.9012 (1.4); 7.6578 (3.3);
7.5369 (0.6); 7.5135 (2.3); 7.4970 (2.7); 7.4811 (1.1); 7.4723 (2.1); 7.4561 (1.0); 7.4327 (0.4); 7.3948 (1.4); 7.3908
(1.6); 7.3748 (2.2); 7.3717 (2.0); 7.2769 (1.7); 7.2666 (2.4); 7.2595 (2.5); 7.2419 (4.7); 3.3370 (35.7); 2.8919 (14.8);
2.7327 (13.4); 2.5694 (16.0); 2.5264 (0.9); 2.5088 (32.2); 2.5044 (41.1); 2.5000 (30.3); 1.2324 (0.4)
I.098: 1H-NMR(400.2 MHz, d6-DMSO):
δ = 8.2490 (3.6); 8.2455 (3.7); 7.9530 (1.8); 7.7513 (3.3); 7.6226 (3.1); 7.6095 (3.1); 7.5493 (0.9); 7.5455 (1.4);
7.5299 (2.2); 7.5272 (2.2); 7.5105 (1.8); 7.5070 (2.2); 7.4966 (1.0); 7.4793 (1.0); 7.4712 (1.1); 7.4622 (0.5); 7.4469
(2.0); 7.4293 (2.2); 7.2918 (4.7); 7.2725 (4.9); 7.2524 (3.9); 3.3341 (123.4); 2.8908 (12.8); 2.7310 (10.8); 2.6762
(0.5); 2.6715 (0.8); 2.5756 (16.0); 2.5251 (2.3); 2.5117 (43.3); 2.5073 (85.8); 2.5028 (110.4); 2.4983 (79.7); 2.4939
(38.7); 2.3341 (0.5); 2.3295 (0.7); 2.3251 (0.5); 1.2344 (0.9); −0.0002 (0.8)
I.099: 1H-NMR(400.2 MHz, d6-DMSO):
δ = 8.4811 (1.9); 8.4779 (2.5); 8.3243 (3.1); 8.3184 (3.2); 7.9961 (1.5); 7.9920 (2.4); 7.9879 (1.5); 7.9810 (0.4);
7.9546 (2.2); 7.8035 (2.6); 7.5922 (0.5); 7.5883 (0.5); 7.5784 (0.5); 7.5743 (0.6); 7.5690 (0.8); 7.5653 (0.8); 7.5551
(0.8); 7.5518 (0.7); 7.5155 (0.6); 7.5042 (0.7); 7.4990 (0.9); 7.4950 (1.4); 7.4862 (0.8); 7.4786 (2.1); 7.4748 (1.1);
7.4584 (1.2); 7.4372 (0.4); 7.3413 (2.5); 7.1472 (0.9); 7.1452 (0.9); 7.1239 (1.6); 7.1025 (0.8); 7.1005 (0.8); 7.0825
(1.8); 7.0623 (1.7); 3.3401 (8.9); 3.0036 (0.5); 2.8921 (16.0); 2.7333 (13.8); 2.7320 (13.3); 2.6854 (0.4); 2.5846
(1.1); 2.5513 (13.3); 2.5270 (0.9); 2.5221 (1.4); 2.5136 (17.2); 2.5091 (34.0); 2.5046 (43.6); 2.5000 (31.0); 2.4955
(14.8); 1.8875 (1.0)
I.100: 1H-NMR(400.2 MHz, d6-DMSO):
δ = 7.9548 (2.2); 7.7786 (3.2); 7.6158 (0.6); 7.6130 (0.6); 7.5985 (0.7); 7.5928 (1.1); 7.5896 (1.1); 7.5791 (1.0);
7.5760 (1.0); 7.5486 (0.8); 7.5307 (0.8); 7.5230 (1.0); 7.5111 (1.0); 7.4956 (4.0); 7.4820 (0.7); 7.4743 (1.6); 7.4703
(1.1); 7.4537 (0.8); 7.2581 (9.2); 7.0951 (1.2); 7.0731 (1.7); 7.0502 (1.0); 7.0056 (2.2); 6.9852 (2.1); 3.3380 (16.4);
2.8923 (15.4); 2.7327 (13.2); 2.5848 (16.0); 2.5270 (0.9); 2.5135 (19.2); 2.5092 (38.0); 2.5047 (49.2); 2.5002 (36.0);
2.4958 (18.1); 2.3314 (0.4); 1.2325 (0.4)
I.101: 1H-NMR(400.2 MHz, d6-DMSO):
δ = 7.9549 (2.3); 7.9375 (1.9); 7.9355 (1.9); 7.9249 (2.0); 7.9229 (1.9); 7.7934 (3.1); 7.6173 (0.5); 7.6009 (0.6);
7.5924 (1.2); 7.5785 (1.1); 7.5613 (3.2); 7.5515 (1.0); 7.5432 (1.0); 7.5328 (0.9); 7.5260 (1.0); 7.5159 (1.1); 7.5123
(1.6); 7.5022 (0.6); 7.4955 (1.5); 7.4921 (1.0); 7.4750 (0.8); 7.4507 (1.4); 7.4389 (2.3); 7.4271 (1.3); 7.0778 (1.1);
7.0556 (1.8); 7.0334 (1.0); 6.9646 (2.2); 6.9442 (2.0); 3.3401 (32.2); 2.8925 (16.0); 2.7330 (13.9); 2.5527 (15.5);
2.5272 (0.9); 2.5136 (17.0); 2.5094 (33.2); 2.5049 (42.6); 2.5004 (30.9); 2.4961 (15.2)
I.102: 1H-NMR(300.2 MHz, CDCl3):
δ = 8.1744 (2.9); 7.6678 (2.8); 7.4724 (0.6); 7.4512 (0.8); 7.4448 (1.4); 7.4236 (1.5); 7.4175 (1.2); 7.3961 (1.2);
7.3862 (2.7); 7.3655 (2.4); 7.3537 (1.3); 7.3334 (1.2); 7.2987 (2.8); 7.0696 (1.1); 7.0572 (1.6); 7.0452 (1.1); 6.9861
(3.4); 6.9582 (4.3); 6.9307 (1.0); 5.4748 (1.7); 5.3342 (3.1); 2.6479 (16.0); 1.6885 (0.6); 1.2881 (0.4); 0.0334 (3.4)
I.103: 1H-NMR(400.2 MHz, d6-DMSO):
δ = 8.2152 (0.6); 8.1948 (1.4); 8.1719 (1.4); 8.1515 (0.6); 7.9554 (2.3); 7.7291 (3.3); 7.6021 (0.5); 7.5878 (0.6);
7.5847 (0.6); 7.5766 (1.3); 7.5623 (1.2); 7.5561 (1.0); 7.5387 (0.9); 7.5307 (1.0); 7.5134 (0.9); 7.5079 (0.5); 7.4944
(1.0); 7.4827 (3.3); 7.4741 (1.8); 7.4573 (1.6); 7.4537 (1.1); 7.4367 (0.8); 7.1321 (1.4); 7.1265 (1.4); 7.1117 (1.4);
7.1061 (1.4); 7.0675 (1.1); 7.0462 (1.9); 7.0249 (1.0); 7.0233 (1.0); 6.9564 (2.2); 6.9361 (2.1); 3.3383 (37.9); 2.8928
(16.0); 2.7338 (13.3); 2.7330 (13.6); 2.5452 (15.9); 2.5278 (0.8); 2.5142 (14.7); 2.5098 (28.7); 2.5053 (36.8); 2.5008
(26.6); 2.4964 (13.0)
I.105: 1H-NMR(300.2 MHz, CDCl3):
δ = 8.5841 (0.8); 8.5782 (0.8); 8.5681 (0.8); 8.5623 (0.8); 7.8375 (1.3); 7.8334 (1.4); 7.5893 (0.7); 7.5837 (0.7);
7.5635 (0.8); 7.5583 (0.8); 7.4234 (0.4); 7.4131 (0.3); 7.4076 (0.3); 7.3960 (1.5); 7.3840 (1.1); 7.3751 (1.1); 7.3680
(0.7); 7.3640 (0.8); 7.3535 (0.8); 7.3470 (0.6); 7.3318 (0.7); 7.2990 (12.3); 7.2141 (1.1); 7.1982 (1.1); 7.1886 (1.0);
7.1726 (0.9); 7.0991 (1.1); 7.0717 (0.9); 6.9485 (0.6); 6.9456 (0.6); 6.9198 (0.9); 6.9177 (0.9); 6.8920 (0.5); 6.8890
(0.5); 5.4046 (1.0); 5.3383 (3.5); 2.5261 (8.5); 1.8852 (0.6); 1.8699 (0.3); 1.5981 (16.0); 1.2833 (0.4); 1.2598 (0.3);
1.2523 (0.4); 1.2412 (0.4); 1.2366 (0.4); 1.2313 (0.4); 1.2255 (0.3); 1.2197 (0.4); 1.0931 (0.4); 1.0872 (0.3); 1.0822
(0.5); 1.0773 (0.4); 1.0665 (0.4); 1.0622 (0.3); 0.9533 (0.4); 0.9417 (0.5); 0.9325 (0.4); 0.9260 (0.4); 0.9209 (0.4);
0.9149 (0.3); 0.7908 (0.3); 0.7861 (0.4); 0.7592 (0.4); 0.0485 (0.7); 0.0377 (16.2); 0.0268 (0.6)
I.106: 1H-NMR(300.2 MHz, CDCl3):
δ = 8.5663 (2.7); 8.5499 (2.7); 8.3545 (4.0); 7.8531 (3.0); 7.8497 (3.0); 7.4367 (0.6); 7.4328 (0.6); 7.4152 (1.2);
7.4062 (1.5); 7.4022 (1.6); 7.3939 (1.2); 7.3878 (3.2); 7.3662 (2.2); 7.3540 (1.6); 7.3392 (1.1); 7.3321 (1.4); 7.3234
(0.5); 7.2989 (2.9); 7.2396 (2.3); 7.2232 (2.3); 7.0603 (2.1); 7.0329 (1.8); 6.9202 (1.2); 6.9175 (1.2); 6.8907 (2.1);
6.8633 (1.1); 6.8606 (1.0); 5.3103 (2.2); 2.5158 (16.0); 1.8536 (0.5); 1.7745 (0.6); 1.7648 (0.6); 1.7470 (0.9); 1.7362
(0.4); 1.7289 (0.6); 1.7190 (0.6); 1.3020 (5.9); 1.2867 (0.8); 1.2747 (4.0); 0.9958 (0.4); 0.9805 (0.7); 0.9748 (0.6);
0.9657 (0.8); 0.9586 (0.8); 0.9522 (0.8); 0.9462 (0.5); 0.9360 (0.9); 0.9306 (0.6); 0.9196 (0.7); 0.9027 (0.9); 0.8868
(0.9); 0.8665 (1.9); 0.8534 (0.8); 0.8440 (1.8); 0.8344 (0.9); 0.8152 (0.7); 0.8039 (0.5); 0.7843 (0.3); 0.7749 (0.3);
0.7568 (0.4); 0.7520 (0.6); 0.7449 (0.9); 0.7346 (0.9); 0.7309 (0.9); 0.7139 (0.8); 0.7079 (0.4); 0.6975 (0.4); 0.1073
(0.4); 0.0311 (3.1)
I.107: 1H-NMR(400.2 MHz, d6-DMSO):
δ = 8.1502 (2.6); 8.1444 (2.6); 7.9555 (0.3); 7.7049 (1.6); 7.6987 (1.5); 7.6836 (1.7); 7.6774 (1.6); 7.6281 (3.0);
7.4557 (0.6); 7.4520 (0.7); 7.4331 (1.6); 7.4177 (4.4); 7.4014 (3.7); 7.3900 (1.1); 7.3738 (0.8); 7.3195 (3.9); 7.3006
(3.4); 7.2842 (0.7); 6.9437 (3.0); 6.5143 (2.6); 6.4929 (2.5); 5.0755 (0.4); 5.0601 (1.2); 5.0447 (1.6); 5.0292 (1.2);
5.0138 (0.4); 3.3443 (29.2); 2.8929 (2.2); 2.7344 (1.9); 2.5958 (13.0); 2.5107 (17.4); 2.5063 (22.1); 2.5019 (16.3);
1.1012 (16.0); 1.0858 (15.7); −0.0002 (1.2)
I.108: 1H-NMR(400.2 MHz, d6-DMSO):
δ = 7.5446 (3.3); 7.5245 (3.5); 7.4467 (1.4); 7.4390 (2.2); 7.4247 (2.8); 7.4146 (1.0); 7.3956 (0.6); 7.3916 (0.8);
7.3758 (1.2); 7.3731 (1.2); 7.3578 (1.0); 7.3536 (1.1); 7.3090 (1.3); 7.3053 (1.3); 7.2899 (2.1); 7.2857 (2.1); 7.2828
(2.1); 7.2801 (2.1); 7.2598 (4.0); 7.2092 (1.2); 7.2061 (1.3); 7.1996 (2.8); 7.1968 (2.8); 7.1910 (1.8); 7.1883 (1.5);
7.1721 (0.7); 7.1693 (0.6); 6.2785 (3.5); 6.2584 (3.5); 3.8565 (0.8); 3.8021 (0.9); 3.7840 (15.4); 3.7247 (16.0);
3.3449 (30.6); 2.8928 (0.4); 2.7342 (0.4); 2.5528 (12.9); 2.5288 (0.5); 2.5155 (8.1); 2.5111 (15.7); 2.5065 (20.0);
2.5020 (14.3); 2.4976 (6.8); −0.0002 (1.1)
I.109: 1H-NMR(300.2 MHz, CDCl3):
δ = 8.7234 (1.4); 8.7175 (1.4); 8.7073 (1.5); 8.7014 (1.4); 7.8748 (2.6); 7.8711 (2.6); 7.6234 (1.5); 7.6174 (1.5);
7.5978 (1.8); 7.5918 (1.7); 7.4557 (0.5); 7.4514 (0.5); 7.4383 (0.6); 7.4326 (0.6); 7.4252 (1.2); 7.4206 (1.3); 7.4018
(2.3); 7.3784 (2.1); 7.3699 (1.4); 7.3565 (1.6); 7.3492 (1.8); 7.3395 (0.6); 7.3292 (2.4); 7.3132 (1.9); 7.2987 (33.8);
7.2909 (1.0); 7.2877 (1.5); 7.0401 (2.0); 7.0125 (1.7); 6.9044 (1.0); 6.9013 (1.0); 6.8754 (1.8); 6.8478 (1.0); 6.8447
(0.9); 5.3375 (2.4); 5.1731 (1.8); 4.1711 (0.3); 4.1475 (0.4); 2.7311 (1.6); 2.7050 (3.1); 2.6798 (2.0); 2.4932 (16.0);
2.0828 (1.6); 1.7608 (0.8); 1.7436 (0.8); 1.7358 (1.5); 1.7185 (1.5); 1.7108 (1.7); 1.6929 (1.8); 1.6853 (1.5); 1.6677
(2.2); 1.6339 (6.5); 1.3206 (0.5); 1.2966 (1.4); 1.2919 (1.1); 1.2729 (0.6); 0.8984 (4.3); 0.8740 (8.8); 0.8494 (3.9);
0.0477 (1.2); 0.0444 (0.8); 0.0369 (39.2); 0.0260 (1.4)
I.110: 1H-NMR(300.2 MHz, CDCl3):
δ = 8.7023 (2.1); 8.6267 (1.4); 8.6101 (1.6); 7.8747 (1.6); 7.4255 (0.7); 7.4217 (0.8); 7.4080 (1.0); 7.4034 (1.3);
7.3812 (1.3); 7.3720 (0.8); 7.3594 (0.9); 7.3517 (0.9); 7.3317 (0.5); 7.2986 (6.6); 7.2417 (1.3); 7.2251 (1.3); 7.0328
(1.3); 7.0051 (1.0); 6.8966 (0.6); 6.8939 (0.6); 6.8675 (1.1); 6.8398 (0.6); 6.8371 (0.5); 5.3362 (0.3); 5.1723 (1.1);
2.5800 (1.0); 2.5544 (2.1); 2.5287 (1.2); 2.4971 (9.0); 1.6451 (16.0); 1.5933 (0.4); 1.5816 (0.4); 1.5684 (0.7); 1.5566
(0.7); 1.5432 (0.7); 1.5317 (0.8); 1.5175 (0.4); 1.5070 (0.6); 0.8874 (2.4); 0.8734 (0.5); 0.8630 (4.8); 0.8494 (0.7);
0.8385 (2.1); 0.0363 (7.6)
I.111: 1H-NMR(300.2 MHz, CDCl3):
δ = 8.6382 (1.7); 8.6212 (1.7); 8.5045 (2.8); 7.8724 (2.8); 7.4533 (0.5); 7.4488 (0.4); 7.4360 (0.6); 7.4306 (0.6);
7.4227 (1.2); 7.4182 (1.4); 7.4054 (1.7); 7.4002 (1.4); 7.3946 (1.5); 7.3837 (1.0); 7.3777 (1.8); 7.3687 (2.5); 7.3636
(2.4); 7.3512 (2.9); 7.3410 (1.5); 7.3300 (1.0); 7.3095 (0.6); 7.2986 (4.5); 7.0150 (1.8); 6.9875 (1.5); 6.8964 (1.0);
6.8676 (1.9); 6.8400 (0.9); 5.3324 (0.4); 5.2119 (1.8); 2.5485 (1.5); 2.5225 (3.0); 2.4965 (16.0); 1.7717 (0.5); 1.6445
(0.4); 1.6242 (0.8); 1.5996 (1.4); 1.5755 (1.6); 1.5530 (1.3); 1.5293 (0.7); 1.2882 (0.7); 0.8873 (4.4); 0.8628 (8.7);
0.8383 (3.8); 0.0326 (4.0)
I.112: 1H-NMR(400.2 MHz, d6-DMSO):
δ = 8.1198 (3.7); 7.9558 (2.2); 7.6779 (2.1); 7.6733 (2.0); 7.6566 (2.2); 7.6519 (2.1); 7.6257 (5.0); 7.5618 (0.9);
7.5582 (0.9); 7.5477 (1.0); 7.5442 (1.1); 7.5387 (1.5); 7.5352 (1.6); 7.5247 (1.4); 7.5219 (1.4); 7.4849 (1.2); 7.4669
(1.4); 7.4593 (1.4); 7.4418 (2.4); 7.4253 (1.6); 7.4214 (2.5); 7.4052 (2.4); 7.4011 (1.5); 7.3847 (1.2); 7.2775 (4.7);
7.1038 (1.6); 7.0825 (2.7); 7.0612 (1.5); 7.0592 (1.6); 7.0490 (3.2); 7.0289 (2.9); 6.6260 (4.2); 6.6248 (4.2); 6.6046
(4.0); 6.6034 (4.0); 4.1646 (2.2); 4.1470 (7.2); 4.1294 (7.3); 4.1119 (2.2); 3.3439 (53.6); 3.3297 (0.6); 2.8931 (15.3);
2.7351 (12.8); 2.7342 (12.9); 2.5514 (23.0); 2.5289 (0.9); 2.5154 (15.8); 2.5111 (30.5); 2.5065 (38.4); 2.5020 (27.2);
2.4975 (12.9); 1.1597 (7.6); 1.1421 (16.0); 1.1245 (7.4); −0.0002 (2.3)
I.113: 1H-NMR(400.2 MHz, d6-DMSO):
δ = 7.9556 (2.0); 7.9329 (3.1); 7.9281 (3.4); 7.9205 (3.3); 7.9157 (3.2); 7.6279 (3.0); 7.6231 (3.0); 7.6096 (3.3);
7.6048 (3.0); 7.5023 (0.5); 7.4838 (5.3); 7.4668 (6.4); 7.4549 (2.7); 7.4506 (3.4); 7.4476 (3.7); 7.4374 (1.4); 7.4286
(0.4); 7.4209 (1.7); 7.4170 (2.4); 7.4006 (2.5); 7.3966 (1.6); 7.3892 (0.5); 7.3802 (1.3); 7.3100 (4.9); 7.3081 (4.9);
7.0371 (6.5); 7.0165 (7.4); 6.9948 (1.6); 6.8469 (3.2); 6.8345 (3.2); 6.8287 (3.2); 6.8163 (3.1); 4.3406 (0.5); 4.3231
(1.8); 4.3140 (0.8); 4.3055 (1.8); 4.2964 (2.4); 4.2879 (0.6); 4.2788 (2.4); 4.2612 (0.7); 4.1629 (0.7); 4.1453 (2.3);
4.1362 (0.6); 4.1277 (2.3); 4.1187 (1.8); 4.1102 (0.8); 4.1010 (1.8); 4.0834 (0.5); 3.3421 (42.2); 2.8924 (13.9);
2.7337 (11.7); 2.5125 (39.9); 2.5112 (39.1); 2.5059 (40.2); 2.5013 (28.9); 2.4969 (14.1); 1.2450 (7.7); 1.2274 (16.0);
1.2098 (7.4); −0.0002 (2.8)
I.114: 1H-NMR(400.2 MHz, d6-DMSO):
δ = 8.4235 (2.0); 7.7982 (0.3); 7.7876 (0.4); 7.6646 (1.0); 7.6606 (1.0); 7.6436 (1.5); 7.6373 (3.2); 7.5782 (0.4);
7.5745 (0.5); 7.5643 (0.5); 7.5605 (0.6); 7.5550 (0.8); 7.5515 (0.8); 7.5410 (0.8); 7.5382 (0.7); 7.4999 (0.6); 7.4813
(0.9); 7.4742 (1.0); 7.4668 (0.8); 7.4622 (0.9); 7.4547 (1.2); 7.4376 (0.9); 7.4337 (1.5); 7.4174 (1.3); 7.4134 (0.8);
7.3969 (0.7); 7.3888 (0.5); 7.3482 (0.6); 7.3401 (2.5); 7.1860 (2.0); 7.1843 (2.0); 7.1651 (1.9); 7.1634 (1.9); 7.1127
(0.9); 7.0914 (1.4); 7.0700 (0.7); 7.0681 (0.7); 7.0202 (1.7); 7.0001 (1.5); 3.6140 (0.5); 3.3428 (26.6); 2.8926 (1.7);
2.7346 (1.4); 2.7336 (1.4); 2.5549 (12.0); 2.5284 (0.5); 2.5149 (8.6); 2.5105 (17.0); 2.5059 (21.8); 2.5013 (15.6);
2.4969 (7.4); 2.3908 (16.0); −0.0002 (1.6)
I.115: 1H-NMR(400.2 MHz, d6-DMSO):
δ = 8.2994 (1.7); 8.2951 (1.8); 8.2872 (1.9); 8.2829 (1.8); 7.9556 (1.9); 7.6277 (2.9); 7.6017 (1.9); 7.5972 (1.9);
7.5827 (2.7); 7.5788 (2.2); 7.5708 (1.0); 7.5421 (0.7); 7.5242 (0.8); 7.5167 (0.9); 7.4992 (0.8); 7.4755 (0.4); 7.4328
(0.6); 7.4124 (1.3); 7.3956 (1.4); 7.3732 (3.0); 7.0386 (1.6); 7.0264 (1.7); 7.0198 (1.6); 7.0076 (1.6); 6.9778 (1.0);
6.9563 (1.7); 6.9346 (0.9); 6.8862 (2.0); 6.8659 (1.8); 3.3449 (36.3); 2.8928 (11.9); 2.7336 (10.4); 2.5105 (35.6);
2.5059 (30.9); 2.5014 (21.7); 2.3711 (16.0)
I.116: 1H-NMR(400.2 MHz, d6-DMSO):
δ = 7.9553 (1.2); 7.8971 (3.3); 7.8843 (3.5); 7.5448 (2.8); 7.4905 (0.8); 7.4865 (0.8); 7.4728 (3.3); 7.4705 (3.1);
7.4539 (3.6); 7.4484 (1.5); 7.4409 (1.0); 7.3234 (1.2); 7.3074 (1.7); 7.3040 (1.7); 7.2494 (3.2); 7.2309 (5.8); 7.2119
(0.8); 7.2094 (0.6); 6.9972 (3.3); 6.9844 (3.2); 3.7653 (16.0); 3.3463 (38.7); 2.8929 (7.7); 2.7340 (6.7); 2.5574
(13.4); 2.5285 (0.5); 2.5150 (9.4); 2.5108 (18.2); 2.5064 (23.3); 2.5019 (17.1); 2.4976 (8.5); −0.0002 (1.0)
I.117: 1H-NMR(400.2 MHz, d6-DMSO):
δ = 8.0681 (0.4); 7.9557 (2.2); 7.8083 (2.9); 7.8049 (3.0); 7.7959 (3.2); 7.7925 (3.1); 7.6170 (0.8); 7.6139 (0.8);
7.6031 (0.9); 7.5994 (1.0); 7.5937 (1.6); 7.5906 (1.8); 7.5798 (1.5); 7.5769 (1.6); 7.5609 (1.7); 7.5511 (4.8); 7.5440
(1.7); 7.5357 (1.6); 7.5179 (1.5); 7.5123 (1.2); 7.5026 (4.9); 7.4436 (1.1); 7.4268 (1.4); 7.4230 (2.4); 7.4063 (2.4);
7.4026 (1.6); 7.3858 (1.2); 7.2805 (4.0); 7.2681 (3.8); 7.0062 (1.7); 6.9849 (2.9); 6.9637 (1.6); 6.9619 (1.6); 6.8756
(3.3); 6.8552 (3.1); 3.8243 (13.9); 3.8176 (13.9); 3.6151 (0.7); 3.3469 (77.9); 2.8938 (16.0); 2.7353 (13.1); 2.7342
(13.4); 2.6875 (1.2); 2.5453 (23.9); 2.5291 (1.0); 2.5244 (1.3); 2.5157 (15.6); 2.5112 (31.5); 2.5067 (41.1); 2.5021
(29.9); 2.4976 (14.6); −0.0002 (0.8)
I.118: 1H-NMR(400.2 MHz, d6-DMSO):
δ = 7.9965 (2.7); 7.9949 (2.9); 7.9906 (2.9); 7.9891 (2.8); 7.9556 (2.3); 7.7187 (3.0); 7.7140 (2.9); 7.6902 (3.6);
7.6132 (0.7); 7.6098 (0.7); 7.5993 (0.8); 7.5954 (0.9); 7.5901 (1.2); 7.5867 (1.2); 7.5761 (1.1); 7.5731 (1.1); 7.5432
(1.0); 7.5252 (1.1); 7.5176 (1.1); 7.4997 (1.1); 7.4946 (0.7); 7.4774 (1.6); 7.4615 (4.8); 7.4405 (1.9); 7.4366 (1.2);
7.4199 (1.1); 7.3482 (0.3); 7.0381 (1.3); 7.0170 (2.4); 6.9957 (1.1); 6.9939 (1.2); 6.9636 (2.6); 6.9434 (2.4); 3.6147
(1.0); 3.3572 (87.1); 2.8934 (16.0); 2.7352 (13.4); 2.7341 (13.6); 2.6871 (0.4); 2.5287 (19.0); 2.5160 (11.6); 2.5116
(22.7); 2.5071 (29.2); 2.5025 (21.0); 2.4980 (10.1); 2.1168 (15.1)
I.120: 1H-NMR(400.2 MHz, d6-DMSO):
δ = 7.9546 (2.3); 7.7349 (2.9); 7.7146 (3.2); 7.6481 (0.6); 7.6446 (0.6); 7.6346 (0.8); 7.6307 (0.7); 7.6252 (1.0);
7.6216 (1.0); 7.6115 (0.9); 7.6082 (0.9); 7.5590 (0.8); 7.5421 (3.6); 7.5287 (3.3); 7.5174 (1.0); 7.5105 (0.6); 7.4919
(0.5); 7.4500 (0.7); 7.4330 (1.0); 7.4295 (1.5); 7.4127 (1.5); 7.4092 (1.0); 7.3922 (0.8); 7.2566 (2.5); 7.2362 (2.4);
7.0348 (1.1); 7.0136 (1.9); 6.9923 (1.0); 6.9907 (1.0); 6.8871 (2.1); 6.8668 (1.9); 3.3370 (20.2); 2.8920 (16.0);
2.7334 (13.7); 2.7321 (13.6); 2.6736 (0.3); 2.6404 (1.2); 2.5169 (20.1); 2.5137 (20.9); 2.5090 (35.8); 2.5044 (45.1);
2.4999 (32.2); 2.4954 (15.4); 2.3312 (0.4); 2.3265 (0.4); 2.3107 (11.3)
I.122: 1H-NMR(400.2 MHz, d6-DMSO):
δ = 8.8811 (2.7); 8.8768 (2.8); 8.6269 (2.5); 8.6239 (2.5); 8.2316 (2.6); 7.9570 (1.6); 7.8226 (3.4); 7.5720 (0.7);
7.5681 (1.0); 7.5518 (3.8); 7.5329 (4.3); 7.4583 (0.5); 7.4417 (0.6); 7.4338 (1.6); 7.4211 (2.2); 7.4012 (5.4); 7.3820
(4.5); 7.3651 (1.1); 7.3621 (0.9); 7.3559 (0.5); 6.9768 (3.2); 6.9746 (3.3); 3.3490 (49.7); 2.8946 (11.0); 2.7363 (9.4);
2.7352 (9.3); 2.5793 (16.0); 2.5306 (0.5); 2.5258 (0.8); 2.5171 (10.0); 2.5127 (20.2); 2.5081 (26.3); 2.5035 (19.2);
2.4991 (9.5); −0.0002 (1.1)
I.123: 1H-NMR(400.2 MHz, d6-DMSO):
δ = 8.8056 (2.7); 8.8014 (2.7); 8.1217 (1.3); 8.1173 (1.3); 8.1015 (1.5); 8.0969 (1.4); 7.9557 (0.4); 7.7464 (3.4);
7.7322 (2.8); 7.7118 (2.5); 7.5481 (0.7); 7.5447 (0.9); 7.5278 (4.1); 7.5091 (4.2); 7.4825 (0.5); 7.4664 (0.6); 7.4587
(1.7); 7.4470 (1.8); 7.4319 (0.9); 7.4237 (1.1); 7.4069 (0.9); 7.3831 (0.4); 7.3739 (1.4); 7.3558 (2.0); 7.3478 (2.4);
7.3390 (1.1); 7.3362 (1.1); 7.3277 (1.9); 7.0993 (3.4); 7.0975 (3.3); 3.3397 (32.9); 2.8927 (3.0); 2.7337 (2.7); 2.5956
(16.0); 2.5278 (0.7); 2.5143 (14.1); 2.5102 (27.3); 2.5057 (34.8); 2.5013 (25.2); 2.4970 (12.3)
I.126: 1H-NMR(400.2 MHz, d6-DMSO):
δ = 8.3300 (3.5); 8.3277 (3.6); 7.9539 (2.2); 7.8696 (2.9); 7.6981 (2.5); 7.6855 (2.5); 7.6189 (0.5); 7.6158 (0.5);
7.6048 (0.5); 7.6017 (0.6); 7.5957 (1.0); 7.5928 (1.0); 7.5819 (0.9); 7.5790 (0.9); 7.5609 (0.8); 7.5434 (0.8); 7.5328
(1.1); 7.5120 (4.5); 7.4951 (1.8); 7.4915 (1.0); 7.4746 (0.7); 7.0773 (1.0); 7.0559 (1.7); 7.0345 (0.9); 7.0325 (0.9);
6.9763 (2.0); 6.9558 (1.8); 3.3359 (24.1); 2.8914 (16.0); 2.7327 (13.0); 2.7315 (13.6); 2.6726 (0.4); 2.5573 (14.5);
2.5261 (1.2); 2.5214 (1.8); 2.5127 (22.2); 2.5082 (45.6); 2.5036 (59.9); 2.4990 (43.4); 2.4945 (21.2); 2.3304 (0.4);
1.2332 (0.5); −0.0002 (0.4)
I.127: 1H-NMR(400.2 MHz, d6-DMSO):
δ = 8.3111 (3.2); 8.3036 (3.4); 8.0455 (1.5); 8.0380 (1.4); 8.0239 (1.6); 8.0164 (1.3); 7.9534 (2.4); 7.7134 (3.0);
7.6440 (0.5); 7.6410 (0.5); 7.6266 (0.7); 7.6181 (1.0); 7.6069 (0.9); 7.5737 (3.4); 7.5544 (0.8); 7.5469 (0.9); 7.5290
(0.8); 7.5056 (0.4); 7.4817 (0.6); 7.4613 (1.4); 7.4445 (1.4); 7.4240 (0.7); 7.0314 (1.1); 7.0098 (1.8); 6.9872 (0.9);
6.9018 (2.0); 6.8814 (1.9); 3.3379 (15.6); 2.8912 (16.0); 2.7315 (14.5); 2.6721 (0.4); 2.5495 (14.1); 2.5077 (47.0);
2.5033 (60.8); 2.4988 (45.2); 2.3299 (0.4); 1.6439 (0.4); 1.2316 (0.4)
I.129: 1H-NMR(400.2 MHz, d6-DMSO):
δ = 8.0973 (2.3); 8.0848 (2.4); 7.9549 (2.3); 7.6710 (3.0); 7.6412 (0.5); 7.6378 (0.5); 7.6272 (0.6); 7.6237 (0.6);
7.6179 (1.0); 7.6147 (1.1); 7.6007 (3.7); 7.5816 (0.8); 7.5640 (0.8); 7.5562 (0.9); 7.5385 (0.8); 7.5332 (0.5); 7.5152
(0.4); 7.5001 (0.7); 7.4830 (3.6); 7.4702 (2.8); 7.4629 (1.6); 7.4591 (1.0); 7.4422 (0.8); 7.0319 (1.1); 7.0106 (1.8);
6.9892 (1.0); 6.9874 (1.0); 6.8973 (2.1); 6.8769 (2.0); 3.3376 (22.7); 2.8924 (16.0); 2.7333 (13.2); 2.7326 (13.4);
2.5497 (14.9); 2.5272 (0.8); 2.5137 (15.5); 2.5093 (30.9); 2.5048 (40.1); 2.5002 (29.0); 2.4958 (14.1); 1.9054 (0.3)
I.130: 1H-NMR(400.2 MHz, d6-DMSO):
δ = 8.0876 (2.3); 7.9553 (1.5); 7.6871 (3.1); 7.6220 (1.3); 7.6171 (1.2); 7.6008 (1.3); 7.5957 (1.3); 7.5451 (0.5);
7.5416 (0.5); 7.5308 (0.6); 7.5275 (0.7); 7.5218 (1.0); 7.5186 (1.0); 7.5078 (0.9); 7.5051 (0.9); 7.4719 (0.7); 7.4539
(0.8); 7.4462 (0.9); 7.4399 (0.9); 7.4279 (1.0); 7.4234 (1.4); 7.4196 (1.6); 7.4035 (1.6); 7.3992 (1.0); 7.3827 (0.8);
7.2354 (2.9); 7.0955 (1.0); 7.0742 (1.7); 7.0721 (1.7); 7.0664 (2.1); 7.0507 (1.3); 7.0463 (1.9); 6.5196 (2.6); 6.4983
(2.6); 5.0423 (0.5); 5.0269 (1.2); 5.0115 (1.7); 4.9961 (1.2); 4.9807 (0.5); 3.3543 (79.8); 2.8935 (10.4); 2.7352 (9.0);
2.7341 (8.4); 2.5539 (14.2); 2.5292 (0.6); 2.5157 (11.0); 2.5113 (21.5); 2.5068 (27.4); 2.5022 (19.7); 2.4978 (9.5);
1.0750 (16.0); 1.0596 (15.8); −0.0002 (0.3)
I.131: 1H-NMR(400.2 MHz, d6-DMSO):
δ = 7.5294 (3.0); 7.5179 (0.6); 7.5094 (4.6); 7.4948 (1.7); 7.4842 (1.0); 7.4681 (0.8); 7.4065 (2.7); 7.3803 (0.6);
7.3642 (3.2); 7.3435 (1.3); 7.3395 (0.8); 7.3231 (0.7); 6.9725 (1.0); 6.9613 (2.0); 6.9515 (1.9); 6.9411 (1.8); 6.9307
(0.9); 6.2784 (3.6); 6.2583 (3.6); 3.7810 (15.6); 3.7030 (16.0); 3.3438 (35.6); 2.5297 (13.3); 2.5150 (8.3); 2.5106
(16.4); 2.5060 (21.2); 2.5014 (15.3); 2.4969 (7.4); −0.0002 (1.2)
I.132: 1H-NMR(400.2 MHz, d6-DMSO):
δ = 7.9922 (3.5); 7.9794 (3.7); 7.9557 (1.9); 7.6053 (0.5); 7.5913 (0.6); 7.5879 (0.6); 7.5794 (1.3); 7.5679 (3.9);
7.5584 (1.2); 7.5475 (3.0); 7.5331 (1.0); 7.5157 (0.8); 7.5103 (0.4); 7.4924 (0.4); 7.4560 (0.7); 7.4392 (0.9); 7.4354
(1.4); 7.4188 (1.5); 7.4150 (0.9); 7.3982 (0.7); 7.0356 (3.5); 7.0228 (3.4); 6.9945 (1.0); 6.9730 (1.7); 6.9517 (0.9);
6.8832 (2.0); 6.8628 (1.9); 3.7983 (16.0); 3.3500 (56.1); 2.8936 (13.0); 2.7349 (11.0); 2.5371 (13.9); 2.5157 (9.7);
2.5113 (18.8); 2.5068 (24.0); 2.5022 (17.2); 2.4978 (8.2)
I.133: 1H-NMR(400.2 MHz, d6-DMSO):
δ = 8.8257 (3.8); 8.6215 (3.4); 8.6184 (3.4); 8.2330 (3.4); 7.9566 (2.4); 7.9092 (4.6); 7.5632 (1.3); 7.5565 (1.0);
7.5465 (2.3); 7.5429 (3.1); 7.5368 (1.7); 7.5331 (1.7); 7.5264 (2.8); 7.5226 (2.8); 7.5060 (1.2); 7.4789 (1.1); 7.4609
(1.2); 7.4533 (1.3); 7.4353 (1.2); 7.4302 (0.8); 7.4119 (0.6); 7.2782 (4.5); 7.1943 (1.6); 7.1727 (2.6); 7.1706 (2.5);
7.1639 (3.3); 7.1492 (1.9); 7.1436 (2.9); 3.3455 (72.8); 2.8942 (16.0); 2.7357 (14.0); 2.7348 (13.4); 2.5350 (21.8);
2.5252 (2.1); 2.5160 (17.0); 2.5117 (33.2); 2.5072 (42.4); 2.5027 (30.8); 2.4983 (15.2); −0.0002 (2.0)
I.134: 1H-NMR(400.2 MHz, d6-DMSO):
δ = 8.7560 (2.5); 8.1017 (1.2); 8.0792 (1.3); 7.9556 (2.3); 7.7948 (3.2); 7.7494 (2.5); 7.7293 (2.3); 7.5759 (0.5);
7.5725 (0.6); 7.5620 (0.6); 7.5584 (0.7); 7.5528 (1.0); 7.5494 (1.1); 7.5359 (1.0); 7.5213 (0.8); 7.5107 (0.9); 7.5046
(1.1); 7.5008 (1.6); 7.4929 (1.0); 7.4844 (2.4); 7.4805 (1.3); 7.4637 (1.3); 7.4441 (0.4); 7.3937 (3.1); 7.1428 (1.1);
7.1212 (1.8); 7.0996 (1.0); 7.0980 (0.9); 7.0527 (2.1); 7.0324 (2.0); 3.3430 (55.4); 2.8931 (16.0); 2.7345 (13.6);
2.7333 (13.5); 2.5543 (15.5); 2.5283 (0.9); 2.5148 (15.0); 2.5105 (29.8); 2.5059 (38.5); 2.5014 (27.9); 2.4969 (13.6)
I.137: 1H-NMR(300.2 MHz, CDCl3):
δ = 8.7345 (1.5); 8.7295 (1.5); 8.7190 (1.6); 8.7141 (1.5); 8.3173 (2.3); 8.3002 (2.3); 7.6450 (2.9); 7.5223 (1.2);
7.5173 (1.2); 7.4964 (2.3); 7.4910 (2.0); 7.4801 (0.7); 7.4727 (1.2); 7.4677 (1.2); 7.4554 (1.1); 7.4499 (1.1); 7.4338
(1.1); 7.4123 (2.8); 7.4013 (1.7); 7.3971 (2.1); 7.3861 (1.5); 7.3802 (1.4); 7.3709 (1.7); 7.3489 (0.5); 7.3149 (1.2);
7.3103 (1.7); 7.3038 (1.5); 7.2986 (4.3); 7.2935 (1.8); 7.2874 (1.0); 7.1173 (2.7); 5.3313 (1.0); 4.1911 (7.0); 2.5891
(16.0); 1.2859 (0.7); 0.0302 (3.8)

### USE EXAMPLES

### Example A: In Vitro Cell Test on Pyricularia oryzae

Solvent: dimethyl sulfoxide
Culture medium: 14.6 g anhydrous D-glucose (VWR),
7.1 g mycological peptone (Oxoid),
1.4 g granulated yeast extract (Merck), QSP 1 liter
   Inoculum: spore suspension

The tested compounds were solubilized in dimethyl sulfoxide and the solution used to prepare the required range of concentrations. The final concentration of dimethyl sulfoxide used in the assay was ≤1%.

A spore suspension of Pyricularia oryzae was prepared and diluted to the desired spore density.

The compounds were evaluated for their ability to inhibit spore germination and mycelium growth in liquid culture assay. The compounds were added in the desired concentration to the culture medium with spores. After 5 days incubation, fungi-toxicity of compounds was determined by spectrometric measurement of mycelium growth. Inhibition of fungal growth was determined by comparing the absorbance values in wells containing the tested compounds with the absorbance in control wells without tested compounds.

In this test, the following compounds according to the invention showed efficacy between 70% and 79% at a concentration of 20 ppm of tested compound: I.015; I.020; I.026; I.030; I.032; I.057; I.079; I.085; I.086; I.087; I.107; I.122; I.123; I.134.

In this test, the following compounds according to the invention showed efficacy between 80% and 89% at a concentration of 20 ppm of tested compound: I.004; I.006; I.010; I.012; I.024; I.033; I.036; I.048; I.052; I.053; I.055; I.066; I.095; I.100; I.106; I.115; I.120; I.131; I.133.

In this test, the following compounds according to the invention showed efficacy between 90% and 100% at a concentration of 20 ppm of tested compound: I.009; I.018; I.022; I.023; I.025; I.027; I.028; I.034; I.035; I.038; I.040; I.044; I.045; I.046; I.047; I.049; I.058; I.059; I.060; I.061; I.062; I.067; I.068; I.069; I.070; I.072; I.074; I.075; I.076; I.077; I.080; I.081; I.083; I.084; I.090; I.091; I.093; I.094; I.096; I.097; I.099; I.101; I.103; I.104; I.105; I.108; I.109; I.110; I.111; I.113; I.117; I.118; I.119; I.125; I.126; I.127; I.129.

### Example B: In Vitro Cell Test on Colletotrichum lindemuthianum

Solvent: dimethyl sulfoxide
Culture medium: 14.6 g anhydrous D-glucose (VWR),
7.1 g mycological peptone (Oxoid),
1.4 g granulated yeast extract (Merck), QSP 1 liter
   Inoculum: spore suspension

The tested compounds were solubilized in dimethyl sulfoxide and the solution used to prepare the required range of concentrations. The final concentration of dimethyl sulfoxide used in the assay was 1%.

A spore suspension of Colletotrichum lindemuthianum was prepared and diluted to the desired spore density.

The compounds were evaluated for their ability to inhibit spore germination and mycelium growth in liquid culture assay. The compounds were added in the desired concentration to the culture medium with spores. After 6 days incubation, fungi-toxicity of compounds was determined by spectrometric measurement of mycelium growth. Inhibition of fungal growth was determined by comparing the absorbance values in wells containing the tested compounds with the absorbance in control wells without tested compounds.

In this test, the following compounds according to the invention showed efficacy between 70% and 79% at a concentration of 20 ppm of tested compound: I.018; I.032; I.040; I.043; I.050; I.059; I.065; I.072; I.073; I.080; I.084; I.085; I.086; I.087; I.088; I.089; I.094; I.095; I.099; I.103; I.107; I.112; I.118; I.120; I.132.

In this test, the following compounds according to the invention showed efficacy between 80% and 89% at a concentration of 20 ppm of tested compound: I.004; I.005; I.009; I.011; I.014; I.016; I.019; I.022; I.023; I.028; I.031; I.036; I.037; I.038; I.042; I.044; I.047; I.048; I.054; I.055; I.063; I.068; I.074; I.075; I.076; I.078; I.083; I.090; I.092; I.100; I.111; I.113; I.115; I.116; I.119; I.122; I.125; I.133.

In this test, the following compounds according to the invention showed efficacy between 90% and 100% at a concentration of 20 ppm of tested compound: I.001; I.002; I.007; I.008; I.010; I.012; I.015; I.024; I.025; I.027; I.033; I.034; I.041; I.045; I.046; I.049; I.051; I.052; I.053; I.056; I.057; I.058; I.060; I.061; I.062; I.066; I.067; I.069; I.070; I.071; I.079; I.081; I.091; I.093; I.096; I.101; I.104; I.105; I.106; I.108; I.109; I.110; I.114; I.117; I.126; I.129; I.131.

### Example C: In Vitro Cell Test on Leptnosphaeria nodorum

Solvent: dimethyl sulfoxide
Culture medium: 14.6 g anhydrous D-glucose (VWR),
7.1 g mycological peptone (Oxoid),
1.4 g granulated yeast extract (Merck), QSP 1 liter
   Inoculum: spore suspension

The tested compounds were solubilized in dimethyl sulfoxide and the solution used to prepare the required range of concentrations. The final concentration of dimethyl sulfoxide used in the assay was ≤1%.

A spore suspension of Leptnosphaeria nodorum was prepared and diluted to the desired spore density. The compounds were evaluated for their ability to inhibit spore germination and mycelium growth in liquid culture assay. The compounds were added in the desired concentration to the culture medium with spores. After 6 days incubation, fungi-toxicity of compounds was determined by spectrometric measurement of mycelium growth. Inhibition of fungal growth was determined by comparing the absorbance values in wells containing the tested compounds with the absorbance in control wells without tested compounds.

In this test, the following compound according to the invention showed efficacy between 80% and 89% at a concentration of 20 ppm of tested compound: I.001.

In this test, the following compounds according to the invention showed efficacy between 90% and 100% at a concentration of 20 ppm of tested compound: I.007; I.008; I.009; I.010; I.011; I.017; I.018; I.041; I.042; I.043; I.044; I.045; I.046; I.047; I.074; I.075; I.076; I.077.

### Example D: In Vivo Preventive Test on Botrytis cinerea (Grey Mould)

| | | |
|---|---|---|
| Solvent: | 5% | by volume of dimethyl sulfoxide |
| | 10% | by volume of acetone |
| Emulsifier: | 1 μL | of Tween ® 80 per mg of active ingredient |

The tested compounds were made soluble and homogenized in a mixture of dimethyl sulfoxide/acetone/Tween® 80 and then diluted in water to the desired concentration.

The young plants of gherkin or cabbage were treated by spraying the tested compounds prepared as described above. Control plants were treated only with an aqueous solution of acetone/dimethyl sulfoxide/Tween® 80.

After 24 hours, the plants were contaminated by spraying the leaves with an aqueous suspension of Botrytis cinerea spores. The contaminated gherkin plants were incubated for 4 to 5 days at 17° C. and at 90% relative humidity. The contaminated cabbage plants were incubated for 4 to 5 days at 20° C. and at 100% relative humidity.

The test was evaluated 4 to 5 days after the inoculation. 0% means an efficacy which corresponds to that of the control plants while an efficacy of 100% means that no disease is observed.

In this test, the following compound according to the invention showed efficacy between 70% and 79% at a concentration of 500 ppm of tested compound: I.001.

In this test, the following compounds according to the invention showed efficacy between 80% and 89% at a concentration of 500 ppm of tested compound: I.017; I.078.

In this test, the following compounds according to the invention showed efficacy between 90% and 100% at a concentration of 500 ppm of tested compound: I.002; I.007; I.008; I.010; I.011; I.014; I.015; I.018; I.022; I.023; I.024; I.041; I.042; I.043; I.044; I.045; I.049; I.056; I.057; I.058; I.059; I.060; I.061; I.062; I.067; I.068; I.069; I.074; I.075; I.076; I.104; I.105; I.109; I.110.

### Example E: In Vivo Preventive Test on Venturia inaegualis (Apples)

| | | |
|---|---|---|
| Solvent: | 24.5 | parts by weight of acetone |
| | 24.5 | parts by weight of dimethyl sulfoxide |
| Emulsifier: | 1 | part by weight of polyoxyethylene sorbitan |
| | | monooleate |

To produce a suitable preparation of the tested compound, 1 part by weight of the tested compound was mixed with the stated amounts of solvent and emulsifier, and the concentrate was diluted with water to the desired concentration.

To test for preventive activity, young plants were sprayed with the preparation of the tested compound at the stated rate of application.

After the spray coating had dried on, the plants were inoculated with an aqueous conidia suspension of the causal agent of apple scab (Venturia inaequalis) and then remained for 1 day in an incubation cabinet at approximately 20° C. and a relative atmospheric humidity of 100%.

The plants were then placed in a greenhouse at approximately 21° C. and a relative atmospheric humidity of approximately 90%.

The test was evaluated 10 days after the inoculation. 0% means an efficacy which corresponds to that of the untreated control, while an efficacy of 100% means that no disease is observed.

In this test the following compounds according to the invention showed efficacy between 90% and 100% at a concentration of 100 ppm of active ingredient: I.041; I.043; I.044.

### Example F: In Vivo Preventive Test on Pyrenophora teres (Barley)

| | | |
|---|---|---|
| Solvent: | 24.5 | parts by weight of acetone |
| | 24.5 | parts by weight of dimethyl sulfoxide |
| Emulsifier: | 1 | part by weight of polyoxyethylene sorbitan |
| | | monooleate |

To produce a suitable preparation of the tested compound, 1 part by weight of the tested compound or compound combination was mixed with the stated amounts of solvent and emulsifier, and the concentrate was diluted with water to the desired concentration.

To test for preventive activity, young plants were sprayed with the preparation of the tested compound at the stated rate of application.

After the spray coating had been dried, the plants were sprayed with a spore suspension of Pyrenophora teres. The plants remained for 48 hours in an incubation cabinet at approximately 20° C. and a relative atmospheric humidity of approximately 100%.

The plants were placed in the greenhouse at a temperature of approximately 20° C. and a relative atmospheric humidity of approximately 80%.

The test was evaluated 8 days after the inoculation. 0% means an efficacy which corresponds to that of the untreated control, while an efficacy of 100% means that no disease is observed.

In this test the following compound according to the invention showed efficacy between 70% and 79% at a concentration of 500 ppm of active ingredient: I.007.

In this test the following compound according to the invention showed efficacy between 80% and 89% at a concentration of 500 ppm of active ingredient: I.067.

In this test the following compound according to the invention showed efficacy between 90% and 100% at a concentration of 500 ppm of active ingredient: I.041.

1. A compound of formula (I) 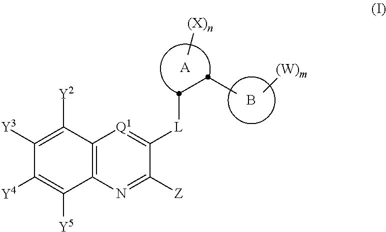
wherein
A is a phenyl or a 5- or 6-membered unsaturated heterocyclyl ring comprising 1, 2 or 3 heteroatoms independently selected in the group consisting of N, O and S, wherein the two points of attachment of the ring A, respectively to the group B and to the group L, are adjacent carbon atoms;
B is a partially saturated or unsaturated 6-membered heterocyclyl ring comprising 1, 2, 3 or 4 heteroatoms independently selected in the list consisting of N, O and S;
Q¹ is CY¹ or N wherein:
Y¹ is selected from the group consisting of hydrogen atom, halogen atom, C₁-C₈-alkyl, C₁-C₈-halogenoalkyl comprising up to 9 halogen atoms that can be the same or different, C₂-C₈-alkenyl, C₂-C₈-halogenoalkenyl comprising up to 9 halogen atoms that can be the same or different, C₂-C₈-alkynyl, C₂-C₈-halogenoalkynyl comprising up to 9 halogen atoms that can be the same or different, C₃-C₇-cycloalkyl, C₄-C₇-cycloalkenyl, hydroxyl, C₁-C₈-alkoxy, C₁-C₈-halogenoalkoxy comprising up to 9 halogen atoms that can be the same or different, formyl, amino, C₁-C₈-alkylamino, di-C₁-C₈-alkylamino, sulfanyl, C₁-C₈-alkylsulfanyl, C₁-C₈-alkylsulfinyl, C₁-C₈-alkylsulfonyl, C₁-C₆-trialkylsilyl, cyano and nitro,
wherein said C₃-C₇-cycloalkyl and C₄-C₇-cycloalkenyl may be substituted with one or more Y^{a} substituents;
Y², Y³, Y⁴ and Y⁵ are independently selected from the group consisting of hydrogen atom, halogen atom, C₁-C₈-alkyl, C₁-C₈-halogenoalkyl comprising up to 9 halogen atoms that can be the same or different, C₂-C₈-alkenyl, C₂-C₈-halogenoalkenyl comprising up to 9 halogen atoms that can be the same or different, C₂-C₈-alkynyl, C₂-C₈-halogenoalkynyl comprising up to 9 halogen atoms that can be the same or different, C₃-C₇-cycloalkyl, C₄-C₇-cycloalkenyl, hydroxyl, C₁-C₈-alkoxy, C₁-C₈-halogenoalkoxy comprising up to 9 halogen atoms that can be the same or different, formyl, amino, C₁-C₈-alkylamino, di-C₁-C₈-alkylamino, sulfanyl, C₁-C₈-alkylsulfanyl, C₁-C₈-alkylsulfinyl, C₁-C₈-alkylsulfonyl, C₁-C₆-trialkylsilyl, cyano and nitro,
wherein said C₃-C₇-cycloalkyl and C₄-C₇-cycloalkenyl may be substituted with one or more Y^{a} substituents;
Z is selected from the group consisting of hydrogen atom, halogen atom, hydroxyl, C₁-C₈-alkyl, C₂-C₈-alkenyl, C₂-C₈-alkynyl, C₂-C₈-halogenoalkynyl comprising up to 9 halogen atoms that can be the same or different, C₁-C₈-alkoxy, C₁-C₈-halogenoalkyl comprising up to 9 halogen atoms that can be the same or different, C₂-C₈-halogenoalkenyl comprising up to 9 halogen atoms that can be the same or different, C₁-C₈-halogenoalkoxy comprising up to 9 halogen atoms that can be the same or different, C₃-C₇-cycloalkyl, C₄-C₇-cycloalkenyl, formyl, C₁-C₈-alkylamino, di-C₁-C₈-alkylamino, sulfanyl, C₁-C₈-alkylsulfanyl, C₁-C₈-alkylsulfinyl, C₁-C₈-alkylsulfonyl, C₁-C₆-trialkylsilyl, cyano and nitro,
wherein said C₃-C₇-cycloalkyl and C₄-C₇-cycloalkenyl may be substituted with one or more Z^{a} substituents;
m is 0, 1, 2, 3 or 4;
n is 0, 1, 2, 3 or 4;
L is CR¹R² or NR³ wherein
R¹ and R² are independently selected from the group consisting of hydrogen atom, halogen atom, C₁-C₈-alkoxy and C₁-C₈ alkyl,
R³ is selected from the group consisting of hydrogen atom, C₁-C₈-alkyl, C₁-C₈-halogenoalkyl comprising up to 9 halogen atoms that can be the same or different, C₂-C₈-alkenyl, C₂-C₈-halogenoalkenyl comprising up to 9 halogen atoms that can be the same or different, C₃-C₈-alkynyl, C₃-C₈-halogenoalkynyl comprising up to 9 halogen atoms that can be the same or different, C₃-C₇-cycloalkyl, C₃-C₇-halogenocycloalkyl comprising up to 9 halogen atoms that can be the same or different, C₃-C₇-cycloalkyl-C₁-C₈-alkyl, C₁-C₈-alkylcarbonyl, C₁-C₈-halogenoalkylcarbonyl comprising up to 9 halogen atoms that can be the same or different, C₁-C₈-alkoxycarbonyl, C₁-C₈-halogenoalkoxycarbonyl comprising up to 9 halogen atoms that can be the same or different, C₁-C₈-alkylsulfonyl, C₁-C₈-halogenoalkylsulfonyl comprising up to 9 halogen atoms that can be the same or different, aryl-C₁-C₈-alkyl and phenylsulfonyl,
wherein said C₃-C₇-cycloalkyl, C₃-C₇-cycloalkyl-C₁-C₈-alkyl, aryl-C₁-C₈-alkyl and phenylsulfonyl may be substituted with one or more R^{3a} substituents;
W is independently selected from the group consisting of halogen atom, hydroxyl, C₁-C₈-alkyl, C₁-C₈-halogenoalkyl comprising up to 9 halogen atoms that can be the same or different, C₁-C₈-alkoxy, C₁-C₈-halogenoalkoxy comprising up to 9 halogen atoms that can be the same or different, C₁-C₈-hydroxyalkyl, C₁-C₈-alkoxy-C₁-C₈-alkyl, C₂-C₈-alkenyl, C₂-C₈-halogenoalkenyl comprising up to 9 halogen atoms that can be the same or different, C₂-C₈-alkynyl, C₂-C₈-halogenoalkynyl comprising up to 9 halogen atoms that can be the same or different, C₃-C₇-cycloalkyl, C₄-C₈-cycloalkenyl, aryl, aryl-C₁-C₈-alkyl, heterocyclyl, heterocyclyl-C₁-C₈-alkyl, aryloxy, heteroaryloxy, arylsulfanyl, arylsulfinyl, arylsulfonyl, heteroarylsulfanyl, heteroarylsulfinyl, heteroarylsulfonyl, arylamino, heteroarylamino, aryloxy-C₁-C₈-alkyl, heteroaryloxy-C₁-C₈-alkyl, arylsulfanyl-C₁-C₈-alkyl, arylsulfinyl-C₁-C₈-alkyl, arylsulfonyl-C₁-C₈-alkyl, heteroarylsulfanyl-C₁-C₈-alkyl, heteroarylsulfinyl-C₁-C₈-alkyl, heteroarylsulfonyl-C₁-C₈-alkyl, arylamino-C₁-C₈-alkyl, heteroarylamino-C₁-C₈-alkyl, aryl-C₁-C₈-alkoxy, heteroaryl-C₁-C₈-alkoxy, aryl-C₁-C₈-alkylsulfanyl, aryl-C₁-C₈-alkylsulfinyl, aryl-C₁-C₈-alkylsulfonyl, heteroaryl-C₁-C₈-alkylsulfanyl, heteroaryl-C₁-C₈-alkylsulfinyl, heteroaryl-C₁-C₈-alkylsulfonyl, aryl-C₁-C₈-alkylamino, heteroaryl-C₁-C₈-alkylamino, formyl, C₁-C₈-alkylcarbonyl, (hydroxyimino)C₁-C₈-alkyl, (C₁-C₈-alkoxyimino)C₁-C₈-alkyl, carboxyl, C₁-C₈-alkoxycarbonyl, carbamoyl, C₁-C₈-alkylcarbamoyl, di-C₁-C₈-alkylcarbamoyl, amino, C₁-C₈-alkylamino, di-C₁-C₈-alkylamino, sulfanyl, C₁-C₈-alkylsulfanyl, C₁-C₈-alkylsulfinyl, C₁-C₈-alkylsulfonyl, C₁-C₆-trialkylsilyl, tri(C₁-C₈-alkyl)silyloxy, tri(C₁-C₈-alkyl)silyloxy-C₁-C₈-alkyl, oxo, cyano and nitro,
wherein said C₃-C₇-cycloalkyl, C₄-C₈-cycloalkenyl, heterocyclyl, aryl and the aryl, heterocyclyl and heteroaryl moieties of the aryl-C₁-C₈-alkyl, heterocyclyl-C₁-C₈-alkyl, aryloxy, heteroaryloxy, arylsulfanyl, arylsulfinyl, arylsulfonyl, heteroarylsulfanyl, heteroarylsulfinyl, heteroarylsulfonyl, arylamino, heteroarylamino, aryloxy-C₁-C₈-alkyl, heteroaryloxy-C₁-C₈-alkyl, arylsulfanyl-C₁-C₈-alkyl, arylsulfinyl-C₁-C₈-alkyl, arylsulfonyl-C₁-C₈-alkyl, heteroarylsulfanyl-C₁-C₈-alkyl, heteroarylsulfinyl-C₁-C₈-alkyl, heteroarylsulfonyl-C₁-C₈-alkyl, arylamino-C₁-C₈-alkyl, heteroarylamino-C₁-C₈-alkyl, aryl-C₁-C₈-alkoxy, heteroaryl-C₁-C₈-alkoxy, aryl-C₁-C₈-alkylsulfanyl, aryl-C₁-C₈-alkylsulfinyl, aryl-C₁-C₈-alkylsulfonyl, heteroaryl-C₁-C₈-alkylsulfanyl, heteroaryl-C₁-C₈-alkylsulfinyl, heteroaryl-C₁-C₈-alkylsulfonyl, aryl-C₁-C₈-alkylamino, heteroaryl-C₁-C₈-alkylamino groups may be substituted with one or more W^{a} substituents;
X is independently selected from the group consisting of halogen atom, hydroxyl, C₁-C₈-alkyl, C₁-C₈-halogenoalkyl comprising up to 9 halogen atoms that can be the same or different, C₁-C₈-alkoxy, C₁-C₈-halogenoalkoxy comprising up to 9 halogen atoms that can be the same or different, C₂-C₈-alkenyl, C₂-C₈-halogenoalkenyl comprising up to 9 halogen atoms that can be the same or different, C₂-C₈-alkynyl, C₂-C₈-halogenoalkynyl comprising up to 9 halogen atoms that can be the same or different, C₃-C₇-cycloalkyl, C₄-C₇-cycloalkenyl, formyl, amino, C₁-C₈-alkylamino, di-C₁-C₈-alkylamino, sulfanyl, C₁-C₈-alkylsulfanyl, C₁-C₈-alkylsulfinyl, C₁-C₈-alkylsulfonyl, C₁-C₆-trialkylsilyl, cyano, nitro and C₁-C₈-hydroxyalkyl, wherein said C₃-C₇-cycloalkyl and C₄-C₇-cycloalkenyl may be substituted with one or more X^{a} substituents;
Z^{a}, R^{3a}, W^{a}, X^{a} and Y^{a} are independently selected from the group consisting of halogen atom, nitro, hydroxyl, cyano, carboxyl, amino, sulfanyl, pentafluoro-λ⁶-sulfanyl, formyl, carbamoyl, carbamate, C₁-C₈-alkyl, C₃-C₇-cycloalkyl, C₁-C₈-halogenoalkyl having 1 to 5 halogen atoms, C₃-C₈-halogenocycloalkyl having 1 to 5 halogen atoms, C₂-C₈-alkenyl, C₂-C₈-alkynyl, C₁-C₈-alkylamino, di-C₁-C₈-alkylamino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-C₁-C₈-alkylcarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylsulfanyl, C₁-C₈-halogenoalkylsulfanyl having 1 to 5 halogen atoms, C₁-C₈-alkylsulfinyl, C₁-C₈-halogenoalkylsulfinyl having 1 to 5 halogen atoms, C₁-C₈-alkylsulfonyl and C₁-C₈-halogeno-alkyl-sulfonyl having 1 to 5 halogen atoms;
and/or a salt, N-oxide, metal complex, metalloid complex and/or optically active isomer and/or geometric isomer thereof;
provided that compound of formula (I) is not:
2-{2-[(6,7-dimethyl-3-oxo-3,4-dihydroquinoxalin-2-yl)methyl]phenyl}-3,5-dioxo-2,3,4,5-tetrahydro-1,2,4-triazine-6-carbonitrile [713527-70-9], and
3,5-dioxo-2-{2-[(3-oxo-3,4-dihydroquinoxalin-2-yl)methyl]phenyl}-2,3,4,5-tetrahydro-1,2,4-triazine-6-carbonitrile [426815-75-0].

2. The compound according to claim 1 wherein A is selected from the group consisting of phenyl, thienyl, pyridinyl and pyrimidyl.

3. The compound according to claim 1 wherein A is selected from the group consisting of A-G1, A-G2, A-G3, A-G4, A-G5, A-G6 and A-G7: 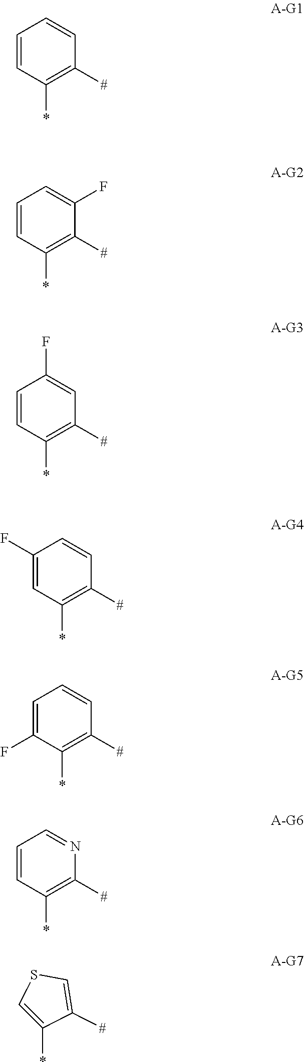
wherein “*” denotes the connection to L and “#” denotes the connection to B.

4. Formula (I) according to claim 1 wherein B is selected from the group consisting of dihydropyranyl, dihydropyridinyl, pyridinyl and pyrimidinyl.

5. The compound according to claim 1 wherein L is CR¹R² wherein R¹ and R² are hydrogen atoms or L is NR³ wherein R³ is hydrogen atom.

6. The compound according to claim 1 wherein X is independently halogen atom.

7. The compound according to claim 1 wherein n is 0 or 1.

8. The compound according to claim 1 wherein W is independently selected from the group consisting of halogen atom, C₁-C₆-alkyl, C₁-C₆-halogenoalkyl comprising up to 9 halogen atoms that can be the same or different, C₁-C₆-alkoxy, C₁-C₆-hydroxyalkyl, C₁-C₆-alkylsulfanyl, C₂-C₆-alkenyl, C₁-C₆-alkoxycarbonyl, C₃-C₇-cycloalkyl, aryl, aryl-C₁-C₆-alkyl, heterocyclyl, carboxyl, tri(C₁-C₆-alkyl)silyloxy-C₁-C₆-alkyl, heteroaryl-C₁-C₆-alkyl, C₁-C₆-alkoxy-C₁-C₆-alkyl, oxo and cyano.

9. The compound according to claim 1 wherein W is independently selected from the group consisting of halogen atom, C₁-C₆-alkyl, C₁-C₆-halogenoalkyl comprising up to 9 halogen atoms that can be the same or different, C₁-C₆-alkoxy, C₁-C₆-alkylsulfanyl, C₃-C₇-cycloalkyl, oxo and cyano.

10. The compound according to claim 1 wherein m is 0, 1 or 2.

11. The compound according to claim 1 wherein Z is a hydrogen atom or a C₁-C₆-alkyl.

12. The compound according to claim 1 wherein Y², Y³, Y⁴ and Y⁵ are independently a hydrogen atom or a halogen atom.

13. The compound according to claim 1 wherein Q¹ is N or CY¹ wherein Y¹ is a hydrogen atom.

14. A composition comprising at least one compound according to claim 1 and at least one agriculturally suitable auxiliary.

15. A method for controlling unwanted phytopathogenic microorganism which comprises applying one or more according to claim 1 or a composition thereof to one or more plants, plant parts, seeds, fruits and/or to soil in which plants grow.
